(19) United States
(12) Patent Application Publication
(10) Pub. No.: US 2010/0035257 A1
(43) Pub. Date: Feb. 11, 2010

(54) METHODS TO DETERMINE THE RESPONSIVENESS TO CISPLATIN TREATMENT

(75) Inventors: Leif W. Ellisen, Newton, MA (US); Chee-Onn Leong, Boston, MA (US)

Correspondence Address: DAVID S. RESNICK, NIXON PEABODY LLP, 100 SUMMER STREET, BOSTON, MA, 02110-2131, US

(73) Assignee: THE GENERAL HOSPITAL CORPORATION, Boston, MA (US)

(21) Appl. No.: 12/444,048

(22) PCT Filed: Oct. 5, 2007
(86) PCT No.: PCT/US07/21433

§ 371 Date: Apr. 2, 2009

Related U.S. Application Data
(60) Provisional application No. 60/850,136, filed on Oct. 6, 2006.

(51) Int. Cl. C12Q 1/68

(52) U.S. Cl. 435/6

(57) ABSTRACT

The present invention relates, generally, to methods to identity subjects responsive to p73/p63 targeting agents such as platinum-based chemotherapy agents such as, but not limited to, cisplatin and cisplatin derivatives and analogues thereof. More particularly, the present invention relates to methods to identify a cancer responsive to a p73/p63 targeting treatment, such as chemotherapeutic agents such as cisplatin, by determining if the cancer expresses and/or has the activity of p63 isoforms such as DNp63 isoforms, and expresses and/or has the activity of p73 isoforms such as TAp73 or DNp73 isoforms. The present invention also relates to methods to identify a cancer unresponsive to a p73/p63 targeting treatment, such as chemotherapeutic agents such as cisplatin by determining if the cancer lacks the expression and/or activity of p63 isoforms such as DNp63 isoforms. The invention further provides kits to determine the expression and/or activity of p63 isoforms such as DNp63 isoforms, and/or the expression and/or activity of p73 isoforms such as TAp73 and/or DNp73 isoforms in a biological sample.

### CROSS REFERENCE TO RELATED APPLICATIONS

This application is a 371 National Phase Entry Application of co-pending International Application PCT/US2007/021433 filed Oct. 5, 2007, which designated the U.S., and claims the benefit under 35 U.S.C 119(e) of U.S. Provisional Patent Application Ser. No. 60/850,136 filed Oct. 6, 2006, the contents of which are incorporated herein by reference in their entirety.

### GOVERNMENT SUPPORT

This invention was made with U.S. Government Support under Grant No. RO1 DE15945 awarded by the National Institutes for Health (NIH). The government of the United States has certain rights in the invention.

### FIELD OF THE INVENTION

The present invention relates generally to the fields of cancer therapy and cancer prevention. More particularly, the present invention relates to methods to determine the level of sensitivity or resistance of tumor cells to p73/p63 targeting treatments, such as chemotherapeutic agents such as cisplatin, and thus the responsiveness of subjects with tumors to such agents. The invention further relates to methods of increasing sensitivity of tumor cells to chemotherapeutic agents and to constructs and compositions for achieving the same.

### BACKGROUND

One of the main problems associated with cancer chemotherapy is that individual subjects with the same histology do not respond identically to a given agent or a given therapeutic protocol. The response range may vary in large proportions, even in chemosensitive tumors such as breast cancer. A number of determinants of drug sensitivity are well known, such as drug dose, drug combinations and schedule of administration, subject age and status, tumor localization etc, but the intrinsic sensitivity of a given tumor is a major factor in which remains difficult to evaluate.

One strategy has been to individualize drug treatment as a function of the sensitivity of tumor cells. Methods to predict how effective a drug may be in a subject are mainly based on in vitro or ex vivo testing of the tumor cells (taken during a biopsy) to a battery of drugs and chemotherapy agents. Such strategies have several limitations; they are often poor predictors of chemosensitivity in vivo, they are time-consuming, and both manually and cost expensive.

The identification of novel cancer subtypes promises to provide more specific, more effective and less toxic therapies. Global gene expression profiling has uncovered previously unrecognized subsets of human breast cancer, including the basal-like or “triple-negative” subset characterized by a lack of estrogen receptor (ER) and progesterone receptor (PR) expression, the absence of Her-2 amplification, and in some studies a high frequency of mutant p53 expression (1, 2). This tumor subset is refractory to commonly used chemotherapeutic agents and therefore is associated with a poor prognosis (1). To date little progress has been made in identifying specific molecular pathways associated with these refractory cancers that may be effectively targeted for therapeutic purposes.

The p53-related transcription factor p63 is an essential regulator of mammary epithelial development. Mice with germline inactivation of p63 exhibited a profound failure of early mammary development, as well as severe developmental abnormalities of the skin, limbs and other ectoderm-derived tissues (3, 4). This same spectrum of deficits is observed in humans inheriting mutations in p63, who exhibit mammary hypoplasia and defects in multiple epithelial tissues (5, 6). In the adult breast, p63 expression is restricted to the basal myoepithelial cell layer, which is known to contribute to proliferation, differentiation, and polarity of mammary epithelia (7, 8). The importance of p63 in mammary development and its potential expression in a subset of breast cancers therefore suggest a possible role for p63 in breast cancer pathogenesis.

A tumor-specific role for p63 in epithelial cells is supported by the observation that p63 expression is increased in up to 80% of primary squamous cell carcinomas (SCCs) of the head and neck, lung, and esophagus (9-13). Recent studies by the inventors demonstrates p63 expression in SCC promotes tumor cell survival through repression of p73-dependent apoptosis (14, 15). Expression of p63 is not detectable in the majority of invasive breast carcinomas, consistent with the fact that most breast tumors exhibit a luminal, rather than basal epithelial phenotype (2, 16). Nevertheless, previous studies have suggested that a fraction of invasive ductal breast carcinomas express p63 protein, with reports ranging from 0-30% of cases (17-20).

Like p53 and the related protein p73, p63 is a sequence-specific DNA binding factor that regulates transcription of critical downstream target genes. All three p53 family members possess a highly homologous DNA binding domain, through which they regulate both shared and distinct subsets of transcriptional targets (21). Expression from two distinct p63 promoters produces protein isoforms that either contain or lack the N-terminal transactivation domain (TAp63 and ΔNp63, respectively). Differential mRNA splicing also gives rise to multiple C-terminal variants (22). ΔNp63α, the predominant p63 isoform expressed in most epithelial cells, exhibits properties of both a transcriptional activator and repressor (23). It is unknown whether primary breast cancers express predominantly TAp63 or ΔNp63 isoforms, since most previous studies have examined total p63 protein or mRNA expression (24).

p73, another p53 family member, may be an important mediator of the response to chemotherapy and other forms of DNA damage in tumor cells (25-29). Specifically, recent studies have demonstrated that cell death following chemotherapy treatment is linked to activation of pro-apoptotic effector genes by TAp73 isoforms (30-32). The implication of these observations for breast carcinoma is unclear. Little data exists regarding the expression TAp73 isoforms in breast cancer, since most prior studies have either not distinguished between isoforms or have focused on expression of N-terminal variants that are structurally and functionally distinct from TAp73 (33, 34). In addition, it is not well established whether endogenous p73 mediates preferential sensitivity to specific chemotherapeutic agents in breast cancer or other tumor types (26, 30-32).

Activation of p73 occurs by multiple pathways. Following DNA damage, p73 isoforms are activated by c-Abl-dependent tyrosine phosphorylation and subsequent p73 isoform stabilization (27-29); AKT-dependent co-activator recruitment (30-32), and Chk1/2-dependent induction of p73 mRNA (51).

Breast cancer is the most frequently diagnosed cancer in women and the second leading cause of cancer deaths in women. According to the World Health Organization, more than 1.2 million people will be diagnosed with breast cancer yearly worldwide, with approximately 213,000 women in the United States are diagnosed with invasive breast cancer each year (Stages I-IV). The chance of developing invasive breast cancer during a woman's lifetime is approximately 1 in 8 (about 13%). Another 62,000 women will be diagnosed with in situ breast cancer, a very early form of the disease. It is estimated that approximately 40,970 women and 460 men die from breast cancer in the United States yearly.

Breast tumors that lack ER/PR expression and Her-2 amplification (referred to herein as “triple-negative subtype”) are among the most refractory of human breast cancers, since they cannot be treated with effective hormonal and Trastuzumab-based therapies. Microarray-based gene expression profiling has revealed that most triple-negative breast carcinomas express proteins associated with mammary basal/myoepithelial cells, including basal cytokeratins 5/6 (16). p63 is highly expressed exclusively in basal cells of the normal mammary gland (46), and in a subset of triple-negative primary tumors. In contrast, p73 is expressed at very low levels in basal epithelia of both the normal epidermis and mammary gland.

It is unclear why the efficacy of cisplatin as a breast cancer treatment is low in some unselected subjects. Thus, identifying which subjects are likely to respond to this cisplatin and other chemotherapy agents targeting p73 will be essential.

There is a significant need in the art for a satisfactory treatment of tumor subsets refectory or non-responsive to commonly used chemotherapeutic agents, (specifically in epithelial cell cancers such as breast, lung, ovarian, brain, colon and prostate cancers), which overcomes the non-responsiveness exhibited by subjects. Such a treatment could have a dramatic impact on the health of individuals, especially older individuals, among whom cancer is especially common, and females whom have a high incidence of breast cancer.

### SUMMARY

The present invention relates to methods to determine the level of sensitivity or resistance of tumor cells to p73/p63 targeting treatments, such as chemotherapeutic agents such as cisplatin, and thus the responsiveness of subjects with tumors to such agents. The present invention is based on discovery that a cancer expressing or having the activity of both a specific isoform of p63, such as DNp63 isoform, and a specific isoform of p73, such as TAp73 or DNp73 is likely to be responsive to a p73/p63 targeting treatment, such as chemotherapeutic agents such as cisplatin or derivatives thereof.

Accordingly, one aspect of the present invention provides methods to detect the expression and/or activity of p63 isoforms, such as DNp63 isoform, and p73 isoforms, such as TAp73 or DNp73 in a cancer, for example a biological sample comprising a cancer obtained from a subject, and if the cancer is determined to express and/or have active p63 isoforms such as DNp63 isoform, and express and/or have active p73 isoforms such as TAp73, the cancer is identified as being likely to be responsive to a p73/p63 targeting treatment such as cisplatin or derivatives thereof.

Another aspect of the present invention relates to methods to detect if a cancer is unresponsive to a p73/p63 targeting treatment such as cisplatin or derivatives thereof. In some embodiments, the methods provides measuring the expression or activity of a DNp63 isoform in a cancer cell, wherein if the cancer cell does not express DNp63, the cancer is identified as being likely unresponsive to a p73/p63 targeting treatment such as cisplatin or derivatives thereof.

The inventors have discovered that the p53 family member p63 controls a pathway for p73-dependent cisplatin sensitivity specific to “triple-negative” tumors. In vivo, the inventors discovered ΔNp63 and TAp73 isoforms were co-expressed exclusively within a subset of triple-negative primary breast cancers that commonly exhibited mutational inactivation of p53. The ΔNp63α isoform was discovered to promote survival of breast cancer cells by binding TAp73 and thereby inhibiting its pro-apoptotic activity. The inventors also discovered that inhibition of p63 by RNA interference led to TAp73-dependent induction of pro-apoptotic Bcl-2 family members and apoptosis. The inventors discovered that breast cancer cells expressing ΔNp63α and TAp73 exhibited cisplatin sensitivity that was uniquely dependent on TAp73.

The inventors also discovered that in tumor cells, ΔNp63α and TAp73 form a complex, and in response to treatment with cisplatin, TAp73 underwent c-Abl-dependent phosphorylation, which promoted dissociation of the ΔNp63α/TAp73 protein complex, releasing TAp73 and allowing it to initiate TAp73-dependent transcription of pro-apoptotic Bcl-2 family members and apoptosis. Accordingly, the inventors have discovered that p63 functions as a survival factor in a subset of cancer cells, such as breast cancer cells and that expression of p63 isoforms in the presence of p73 isoforms confers cisplatin sensitivity in these cells, for example breast cancer cells that are triple-negative cancers.

Without being bound by theory, platinum-based chemotherapy drugs, such as cisplatin are not effective in the vast majority of subjects affected with cancers, in particular certain subtypes of breast cancer. The inventors have discovered herein that subtypes of cancer cells are responsive to cisplatin due to the presence of a p63 isoform, in particular a DNp63 isoform in cells also expressing an isoform of p73.

The inventors have discovered that p73 isoforms are inactivated by binding and sequestration by ΔNp63 isoforms, preventing p73 downstream signaling of pro-apoptotic pathways. Chemotherapy compounds such as cisplatin which function through p73/p63 pathways, result in the release of p73 isoforms from a p73:ΔNp63 complex, thus attenuating ΔNp63-mediated inactivation or suppression of p73 isoforms, and enable the released p73 isoforms to mediate p73 pro-apoptotic signaling and cell death. Accordingly, the inventors gave discovered that tumor cells co-expressing both p63 isoforms and p73 isoforms, in particular DNp63 isoforms and TAp73 isoforms respectively, increases the tumor cells susceptibility to chemotherapeutic drugs that function through p73/p63 targeting pathways.

Accordingly, the inventors have discovered the presence of DNp63 and TAp73 can be used as a biomarker to identify cancer cells responsive to p73/p63 targeting treatments, such as cisplatin. The inventors have demonstrated that determining the molar ratio of p63 to p73 is one way to identify if a cancer cell is above a threshold level to be identified as being positive for the biomarker, and thus identify cancers responsive to a p73/p63 targeting treatment, such as cisplatin. In some embodiments, the inventors have discovered that cells with a DNp63:p73 molar ratio of 1:1 identifies a cancer likely to be responsive to a p73/p63 targeting treatment. In particular embodiments, if the level of DNp63 is greater then the level of p73 isoform the cancer is more likely to be responsive to a p73/p63 targeting treatment, for example, if the level of DNp63 is at least 1.2 fold higher, or at least 1.5 fold higher than the level of the p73 isoform, then the cancer is identified as being responsive to a p63/p73 targeting treatment such as cisplatin. In some embodiments, a cancer cell with a DNp63:TAp73 molar ratio of >2 is considered to be positive for the biomarker and identifies cancer cells which are sensitive (or responsive) to a p73/p63 targeting treatments, such as cisplatin. Without being bound by theory, the threshold to identify if a cancer cell responsive to a p73/p63 targeting treatment is level of DNp63 which is sufficient to sequester the p73 isoforms, such as TAp73 or DNp73 isoforms in the cell, and thus cisplatin will release of significant amounts of TAp73 isoforms from the TAp73:DNp63 complex.

Accordingly, in another embodiment, the inventors have discovered the absence of expression or activity of DNp63 isoforms in a cancer cell can be used as a biomarker to identify cancer cells unresponsive to a p73/p63 targeting treatment such as cisplatin. In such an embodiment, the method comprises measuring the expression and/or activity of at least one isoform of DNp63 in at least one cancer cell, wherein the absence of expression and/or activity of a DNp63 isoforms identifies the cancer as being more likely to be unresponsive to cisplatin or a derivative thereof, as compared to a cancer wherein the expression or the activity of DNp63 is detected.

In another embodiment, a cancer cell is identified as being unresponsive to a p73/p63 targeting treatment if the level of expression of DNp63 in the cancer cell below a level DNp63 of a reference level, for example where a reference level is, but not limited to a level of DNp63 in a cancer cell responsive to a p73/p63 targeting treatment such as cisplatin.

In one aspect of the present invention provides a method of identifying the likelihood of a cancer to be responsive to a p73/p63 targeting treatment, such as for example cisplatin or cisplatin derivatives, where the expression and/or activity of at least one isoform of p73 such as TAp73 or DNp73, and at least one isoform of DNp63 is measured in at least one cancer cell, for example a cancer cell obtained from a subject, and if such a cancer cells has the expression and/or activity of both a p73 and DNp63 isoform, the cancer is identified as being more likely to be responsive to a p73/p63 targeting treatment, such as for example cisplatin or cisplatin derivatives, as compared to a cancer wherein the expression or the activity of only a p73 or only DNp63 isoform is detected. In some embodiments, the TAp73 isoforms is any p73 isoform, for example TAp73 or DNp73, and in some embodiments, the DNp63 isoform is any p63 isoform.

Another aspect of the present invention provides a method for treating cancer in a subject, for example the methods comprise measuring the expression and/or activity of at least one DNp63 isoform in a biological sample comprising cancer cells, for example cancer cells obtained from the subject, and measuring the expression and/or activity of at least one p73 isoform in the biological sample comprising cancer cells obtained from the subject. In such an embodiment, the method comprises comparing the presence of the expression or activity of the DNp63 isoform with the presence of expression or activity of the p73 isoform, and a cancer is identified to be more likely to be responsive to a p73/p63 targeting treatment, such as cisplatin or cisplatin derivatives if the biological sample comprises the expression and/or activity of both p73 and DNp63 isoforms as compared to expression and/or activity of only p73 or only p63. In some embodiments, the method further comprise administering to a subject a p73/p63 targeting treatment, such as cisplatin or cisplatin derivatives, to a subject identified as being responsive to a p73/p63 targeting treatment or administering to a subject an anti-cancer agent other than a p73/p63 targeting treatment to a subject identified as not being responsive to a p73/p63 targeting treatment.

In some embodiments, to provide a greater accuracy of determining the likelihood of a cancer being responsive to a p73/p63 targeting treatment such as for example cisplatin or cisplatin derivatives, levels of expression and/or activity of at least one isoform of p73, such as TAp73 or DNp73, and at least one isoform of p63, such as DNp63 can be measured. In such embodiments, one can compare the level of the expression or activity of at least one isoform of p73 and at least one isoform of p63, and if a cancer cell comprises a higher level of expression and/or activity of the p63 isoform, such as a DNp63 isoform, as compared to level of expression and/or activity of a p73 isoform, such as a TAp73 or DNp73 isoform, then the cancer is identified as more likely to be responsive to a p73/p63 targeting treatment such as cisplatin or cisplatin derivatives as compared to cancers where the level of expression and/or activity of DNp63 is below the level of expression and/or activity of TAp73. In some embodiments, a further increase in accuracy is achieved if a cancer cell is identified to have at 1.2 fold higher, or at least 1.5 fold higher or at least twice as high levels of expression and/or activity of DNp63 isoform as compared to level of expression and/or activity of the p73 isoform, which identifies a cancer as more likely to be responsive to a p73/p63 targeting treatment, such as cisplatin or cisplatin derivatives, as compared a cancers wherein the level of expression and/or activity of DNp63 is below the level of expression and/or activity of p73 isoforms such as TAp73 or DNp73.

In some embodiments, the methods of the present invention measure protein expression or levels or protein expression, for example by methods such as, but not limited to immunoblot analysis, immunohistochemical analysis; ELISA, isoform-specific chemical or enzymatic cleavage, or mass spectrometry. In some embodiments, protein expression is measured by contacting the cancer cell with at least one protein binding agent, for example but not limited to; antibodies; recombinant antibodies, chimeric antibodies, tribodies, midibodies, protein-binding agents, small molecule, recombinant protein, peptides, aptamers, avimers and derivatives or fragments thereof.

In alternative embodiments, the present invention measures gene transcript expression and/or mRNA expression, or levels of gene transcript expression such as mRNA, for example by methods such as, but not limited to reverse-transcription polymerase chain reaction (RT-PCR) or by quantitative RT-PCR (QRT-PCR) reaction.

In some embodiments of the present invention, the expression or activity of p73 isoforms such as TAp73 or DNp73 isoforms and p63 isoforms such as DNp63 isoforms are measured in vitro and in alternative embodiments, measure are performed in vivo by any appropriate technique commonly known in the art.

In some embodiments, a cancer cell useful to measure expression or activity of p63 isoforms such as DNp63 and p73 isoforms such as TAp73 or DNp73 is present in a biological sample. In some embodiments, a cancer cell can be obtained from a tissue sample; tumor sample; tumor cell; biopsy sample; ex vivo cultivated sample; ex vivo cultivated tumor sample; surgically dissected tissue sample, cancer sample, or primary ascite sample.

Another aspect of the present invention relates to a method to identify if a cancer is likely to be responsive a p73/p63 targeting treatment, such as cisplatin or cisplatin derivatives, comprising measuring the expression of at least one p73 isoform such as TAp73 or DNp73 in the cancer by performing quantitative RT-PCR, and measuring the expression of at least one DNp63 isoform in the cancer by performing quantitative RT-PCR, and identifying if a cancer is likely to be responsive to a p73/p63 targeting treatment such as cisplatin, as a cancer cell identified to have a level of at least one isoform of DNp63 the same or greater than the level of at least one isoform of p73 as compared to a cancer cell where the level of at least one isoform of DNp63 is absent or below the level of at least one isoform of p73.

Another aspect of the present invention relates to a method for increasing sensitivity of a tumor cell to a p73/p63 targeting treatment, the method comprising administering to the cell an effective amount of an antagonist of a p63 isoform or DNp63 isoform.

Another aspect of the present invention relate to kits to measure the expression and/or activity of a p63 isoform such as DNp63 and a p73 isoform such as TAp73 or DNp73. In some embodiments, a kit can comprise at least one primer pair designed to anneal to the nucleic acid regions of DNp63 isoforms and at least one primer pair designed to anneal to the nucleic acid regions of p73 isoforms.

In another embodiment, a kit can comprise least one primer pair designed to anneal to the nucleic acid regions of DNp63 isoforms and at least one primer pair designed to anneal to the nucleic acid regions of p73 isoforms such as TAp73 or DNp73 isoforms. In another embodiment, a kit can comprise at least one primer pair designed to anneal to the nucleic acid regions of DNp63 isoform, and a probe designed to anneal to DNp63 isoforms and at least one primer pair designed to anneal to the nucleic acid regions of p73 isoform, and a probe designed to anneal to p73 isoforms. In another embodiment, a kit can comprise at least one probe designed to anneal to the messenger RNA of p63, or isoforms of p63 at least one probe designed to anneal to the messenger RNA of p73, or isoforms of p63.

In some embodiments, a kit can optionally further comprise products and reagents to carry out QRT-PCR amplification reactions and/or instructions. In some embodiments, the kits can comprise at least two primers selected from, for example but not limited to, SEQ ID NO: 9, SEQ ID NO: 10, SEQ ID NO:11, SEQ ID NO:12, SEQ ID NO: 13, SEQ ID NO:14, SEQ ID NO:15, SEQ ID NO:16, SEQ ID NO: 17 and SEQ ID NO:18 or variant sequences thereof.

Another aspect of the present invention relate to kits to measure the protein expression and/or activity of a p63 isoform such as DNp63 and a p73 isoform such as TAp73 or DNp73. In some embodiments, a kit can comprise at least one protein-binding agent designed to bind to p63 protein isoforms, such as DNp63 informs, and at least one protein-binding agent designed to bind to p73 protein isoforms, such as TAp73 or DNp73 isoforms. In some embodiments, a protein-binding agent useful in such a kit can bind to p63 isoforms such as DNp63 and/or p73 isoforms such as TAp73 or DNp73 in a complex comprising at least one DNp63 isoform complexed with at least one isoform of p73. In some embodiments, protein-binding agents useful in the kits are, for example but are not limited to; antibodies; recombinant antibodies, chimeric antibodies, tribodies, midibodies, protein-binding agents, small molecule, recombinant protein, aptamers, avimers and derivatives or fragments thereof. In some embodiments, a protein-binding agent is labeled with a detectable label or detectable marker. In some embodiments, a kit can optionally further comprise products and reagents to carry out the protein binding reactions and/or instructions.

Another aspect of the present invention provides a method for treating cancer in a subject comprising measuring the presence or expression and/or activity of at least one isoform of p73, such as a TAp73 or DNp73 isoform, and at least one isoform of p63, such as a DNp63 isoform, in a cancer cell obtained from the subject, wherein a clinician then reviews the results and if the results indicate the presence of expression and/or activity of an isoform of p63 such as a DNp63 isoform and the presence of expression and/or activity of an isoform of p73 such as a TAp73 or DNp73 isoform in the cancer cell, then clinician directs the subject to be treated with a p73/p63 targeting treatment, such as cisplatin or derivative thereof. In some embodiments, if the clinician reviews the results and if the results indicate that level of expression and/or activity of an isoform of p63 such as DNp63 is the same, or a higher level of as compared to the level of expression and/or activity of an isoform of p73, such as TAp73 or DNp73 in the cancer cell, then clinician directs the subject to be treated with a p73/p63 targeting treatment or cisplatin or derivative thereof.

One aspect of the present invention provides methods to determine the likelihood of effectiveness of a p73/p63 targeting treatment in a subject affected with, or at risk of cancer. In some embodiments, the method comprises determining expression and/or activity of p63 isoforms and p73 isoforms in a biological sample from a subject or subject. In some embodiments, subjects where the biological sample is found to express and/or have activity of both a DNp63 isoform and a 73 isoform, such as TAp73 or DNp73, indicates a p73/p63 targeted treatment is likely to be effective in the subject. For example, if the biological sample is found to express and/or have a greater activity of the DNp63 isoform at least 1.2 fold more, or at least 1.5 fold more, or at least two fold more as compared to a p73 isoform, such as TAp73 or DNp73 isoform it indicates a p73/p63 targeted treatment is likely to be effective in the subject. However, if the biological sample is found to express only a p63 isoform such as DNp63, or only a p73 isoform such as TAp73, then it indicates a p73/p63 targeted treatment is not likely to be effective in the subject. Without being bound by theory, the inventors have discovered that p73 isoforms such as TAp73 or DNp73 isoforms, in the absence of DNp63 isoforms are likely to be inactivated by a p63-independent mechanism and thus p73 isoforms such as TAp73 or DNp73 are not likely to be activated by cisplatin or derivatives thereof.

In some embodiments, the level of expression and/or activity of p63 isoforms and p73 isoforms are determined in a biological sample from a subject having cancer, suspected of having cancer or at risk of developing cancer. In some embodiments, the biological sample comprises a cancer cell obtained from a subject. In some embodiments, the biological sample is a tissue sample such as a biopsy tissue sample, and ex vivo cultivated biopsy tissue sample, a surgically-dissected tissue sample, or an ex vivo cultivated surgically-dissected tissue sample.

Accordingly, one aspect of the present invention provides methods to determine the likelihood of effectiveness of a p73/p63 targeting treatment in a subject at risk of developing, or affected with cancer. The methods as disclosed herein is useful for determining the effectiveness of all p73/p63 targeting treatments, particularly anti-cancer p73/p63 targeting treatments, as well as all anti-cancer drugs such as cisplatin, cisplatin compounds, cisplatin derivatives or molecules or drugs having a skeleton of cisplatin, or cisplatin mimetics. In some embodiments, a p73/p63 targeting treatment can be a cisplatin analogue, for example but not limited to carboplatin and oxaliplatin, or derivatives thereof. In another embodiment, p73/p63 targeting treatments may be any agent, entity, or small molecule mimicking the mechanism of cisplatin, or any agent, entity, or small molecule which functions, entirely or in part, through the p73/p63 pathway.

In another embodiment, the method comprises quantitatively determining the level of expression of DNp63 isoforms and p73 isoform in a biological sample obtained from the subject, as an exemplary example, a biological sample from a tumor biopsy such as invasive breast carcinoma cells. In another embodiment, levels of DNp63 isoform and p73 isoform expression in a biological sample obtained from the subject are compared with the expression level DNp63 isoform and p73 isoform levels in specimen-matched normal (non-tumor) luminal epithelial cell, using isoform-specific quantitative RT-PCR assays.

In some embodiments, the methods provide determination of the expression level of DNp63 isoform and p73 isoforms using any method commonly known by persons of ordinary skill in the art, such as but not limited to; quantitative reverse transcriptase polymerase chain reaction (QRT-PCR), amplifying the copy-DNA or copy nucleic acid (cDNA) sequence derived from the transcription of messenger (mRNA) transcript encoding either p63 isoforms or p73 isoforms.

In some embodiments, the expression level of p63 and p73 can be determined using isoform-specific QRT-PCR using any method commonly known by persons of ordinary skill in the art, amplifying the cDNA sequence derived from the mRNA transcript encoding an isoform of DNp63 or an isoform of p73. In one embodiment, the isoform of p63 is a DNp63 isoform and the isoform of p73 is a TAp73 or DNp72 isoform.

In another embodiment, the detection of the expression level of DNp63 isoforms or p73 isoforms comprises contacting the DNp63 or p73 mRNA transcripts with at least one nucleic acid probe specific for isoforms of DNp63 or isoforms of p73. The DNp63 or p73 probes preferentially hybridize with mRNA sequences for isoforms of DNp63 or isoforms of p73 respectively under selective hybridization conditions. Isoform-specific DNp63 or p73 probes preferentially hybridize with mRNA sequences for the respective isoforms of DNp63 or p73.

In another embodiment, the methods provide determination of the protein expression level of p63 isoforms and protein expression levels of p73 isoforms, for example protein level of DNp63 isoforms and TAp73 or DNp73 isoforms. The methods as disclosed herein encompass any method commonly known by persons of ordinary skill in the art to determine and measure the protein expression of p63, for example DNp63 and p73, such as TAp73 or DNp73.

In some embodiments, methods to determine protein expression use protein binding molecules or probes, for example probes that bind to the proteins of specific isoforms of DNp63 or p73. Probes useful in the methods of the present invention to detect levels of p63 isoform proteins and levels of p73 isoform proteins can be any probe that preferentially binds to isoforms of p63 such as DNp63 and/or isoforms of p73 such as DNp73. For example, such probes include, but are not limited to antibodies, antibody fragments, chimeric antibodies, humanized antibodies, human antibodies, binding proteins, recombinant binding proteins, binding protein fragments or hybrid binding proteins, hybrid binding protein fragments, aptamers, small molecules, avimirs and fragments and derivatives thereof.

In one embodiment, one protein-binding probe can bind to a DNp63a isoform protein and another protein-binding probe can binds to a p73 isoforms such as TAp73 or DNp73 isoforms. In some embodiments, a probe can be an antibody or protein-binding molecule that preferentially binds to isoforms of DNp63 and another probe can be an antibody or protein-binding molecule that preferentially binds to p73 isoforms such as TAp73 or DNp73 isoforms. In some embodiments, the probes can be used to detect DNp63 isoforms and p73 isoforms simultaneously, or one at a time, sequentially, or in any order.

For example, a probe that can detect DNp63 and p73 isoforms simultaneously is can be a probe comprising a DNp63 protein-binding probe conjugated to a p73 protein binding probe that recognizes TAp73 or DNp73. Such multi-protein binding probes are often, but not exclusively, protein or polypeptides conjugated together, for example by conjugation methods commonly known in the art, and are for example, conjugation by chemical means, covalent bonds, linkers and the like. In some embodiments the conjugation may be protein fusion, the methods of which are well known in the art.

In some embodiments, a multi-protein binding probe useful in the methods as disclosed herein, for example a multi-protein binding probe capable of detecting p63 isoforms such as DNp63 isoforms and p73 isoforms, such as TAp73 or DNp73 isoforms is an avimer. The multivalent binding interactions are characterized by the concurrent interaction of multiple ligands with multiple ligand binding sites on one or more cellular receptors. Multivalent interactions can differ from individual monovalent interactions by binding with different binding affinities as well as enhanced binding specificity and affinity to more than one protein. An avimer is an example of a multi-binding probe useful in the method as disclosed herein, which relates to a peptide agent which is capable of binding to one or more sites or one or more proteins, for example one or more proteins in a complex.

Avimers are multi-domain proteins with multiple binding properties and are comprised typically of multiple independent binding domains linked together, such as a binding domain for p63 isoforms such as DNp63 and a binding affinity for p73 isoforms such as TAp73 or DNp73. Avimers have improved affinity and specificity for multiple proteins, such as DNp63 isoforms and TAp73 isoforms herein as compared to conventional single epitope binding probes. In some embodiments, an avimer is a useful agent in the measurement of the expression and/or activity of p63 isoforms such as DNp63 and a binding affinity for p73 isoforms such as TAp73, or for the detection of a complex comprising, for example, DNp63 and p73, where the avimer is a protein or polypeptide that can bind simultaneously to DNp63 and TAp73, a process known as multi-point attachment in the art.

In some embodiments, the type of probes used to detect the protein expression level DNp63 isoforms does not need to be the same type of probe used to detect the protein expression level of p73 isoforms, for example any combination of different types of probes can be utilized. Further, in some embodiments nucleic acid probes can be used to determine the nucleic acid expression of p63, and protein-binding probes can be used to determine the protein expression of p73 isoforms, provided such a combination is sufficient to enable the detection of both p63 and p73 isoforms. In some embodiments, the presence of a p73 isoform protein or gene expression (such as a TAp73 or DNp73 isoform protein or gene expression) detected in the presence of the protein or expressed nucleic acid of an isoform of DNp63, indicates the p73/p63 targeting treatment is likely to be effective. In particular, a level of p73 isoform protein or gene expression (such as a TAp73 or DNp73 isoform protein or gene expression) detected in the presence of the protein or expressed nucleic acid of an isoform of DNp63, where the level of p63 or DNp63 is greater, for example in some instances at least 1.2-fold greater, and in some instances 1.5-fold greater or in some instances at least two-fold molar ratio greater than that of p73 or TAp73 or DNp73 indicates the p73/p63 targeting treatment is likely to be effective.

In some embodiments, the present invention further provides methods to detect the presence of both DNp63 and p73 using probes which specifically bind to a complex comprising isoforms of p63 and isoforms of p73, for example probes which bind to a DNp63:p73 complex or a DNp63:TAp73 or DNp63:DNp73 complex. The presence of a complex comprising DNp63 isoforms and p73 isoforms such as TAp73 or DNp73 indicates the likelihood of the p73/p63 targeting treatment will be effective.

In some embodiments, probes useful in the methods as disclosed herein to measure the nucleic acid expression level of p63 isoforms such as DNp63 isoforms, and the nucleic acid expression level of p73 isoforms such as TAp73 or DNp73 isoforms are nucleic acid probes or nucleic acid analogue probes of the equivalent of about 500 nucleotide bases, about 100 nucleotides bases, and about 50 or about 25 bases or fewer in length. In some embodiments, nucleic acid probes or nucleic acid analogue probes can be composed of DNA, RNA, or nucleic acid analogues such as peptide nucleic acid (PNA), pseudo-complementarily-PNA (pc-PNA), locked nucleic acid (LNA) or derivatives thereof. In some embodiments, nucleic acid probes or nucleic acid analogue probes can further comprise a detectable label, such as, for example, a florescent or enzymatic label.

Another aspect of the present invention provides methods for treating and/or preventing a subject affected with or at risk of developing cancer, the method comprising measuring the levels of DNp63 and p73 assessing if the subject is responsive to a p73/p63 targeting treatment, and if the subject is identified to be responsive to such a treatment, then the subject is administered a p73/p63 targeting treatment. In some embodiments, the method comprises determining expression level and/or activity of p63 isoforms such as DNp63 isoforms and determining expression level and/or activity of p73 isoforms such as TAp73 or DNp73 isoforms in biological sample from a subject, for example a biological sample comprising a tumor from a subject. The detection of the expression and/or activity of p73 isoforms such as TAp73 isoforms and the detection of the expression and/or activity of p63 isoforms such as DNp63 isoforms indicate a p73/p63 targeting treatment is likely to be an effective treatment or preventative treatment in the subject. In particular, if the molar ratio of the level of expression of the p63 isoform such as the level of the DNp63 isoform is greater as compared to the level of expression of the p73 isoform, such as TAp73 or DNp73, then the p73/p63 targeting treatment is likely to be an effective treatment or preventative treatment in the subject, and thus the subject is likely to be responsive to a p73/p63 targeting treatment. For example, of the level of DNp63 is at least 1.2 fold, or at least 1.5 fold, or at least 2-fold greater than the level of the p73 isoform, such as the level of TAp73 or DNp73 isoform, then a p73/p63 targeting treatment is likely to be an effective treatment for cancer in a subject as the cancer is identified as being likely to be responsive to a p73/p63 targeting treatment such as cisplatin.

A subject identified as being likely to be responsive to a p73/p63 targeting treatment can be treated with a therapeutically effective amount of a p73/p63 targeting treatment, such as cisplatin or derivatives or analogues thereof, either alone or in combination with other therapeutic and/or anti-cancer drugs.

In some embodiments, a p73/p63 targeting treatment is a chemotherapy agent or a platinum-based chemotherapy agent, for example but not limited to platinum-based chemotherapy agents such as cisplatin (also known in the art as cis-diaminedichloroplatinuim (II), cis-DDP, CDDP), cisplatin compounds, cisplatin metabolites, derivatives or analogues thereof having a skeleton similar to cisplatin. Analogues of cisplatin, for example include, but are not limited to, carboplaitin (cis-diamine[1,1-cyclobutnaedicarboxylate(2-)-O,O′-platinum(II)) and oxaliplatin (cis-L-diaminocyclohexane oxalotoplatinum (II). Cisplatin derivatives include, for example but not limited to, those set forth in U.S. Patent Application No: US2006/0142593 which is specifically incorporated herein in its entirety by reference. In some embodiments, p73/p63 targeting treatments also encompass any agent, such as small molecules that function on any part of the p73/p63 pathway.

Another aspect of the present invention provides to methods for treating and/or preventing a subject affected with or at risk of developing cancer, the method comprising measuring the levels of DNp63 isoforms and p73 isoform, such as TAp73 or DNp73 and assessing if the subject is responsive to a p73/p63 targeting treatment as determined by the methods as disclosed herein, and if the subject is identified to express both DNp63 and p73 isoforms such as TAp73 and/or DNp73, then the subject is administered an agent which inhibits DNp63. In some embodiments, inhibiting the expression or activity of DNp63 isoforms, significantly reduces DNp63-mediated inhibition of p73 isoforms, allowing for pro-apoptotic signaling of p73. In some embodiments, the method comprises introducing into tumor cells an effective amount of one or more p63 isoform antagonists or inhibitory agents, for example an effective amount of antagonists or inhibitor of a DNp63 isoform.

In some embodiments, antagonists to isoforms of p63 and/or DNp63 isoforms can be any agent that inhibits the expression or protein activity of p63 or DNp63, and include for example, but are not limited to agents such as antibodies, antibody fragments, small molecules, peptides, proteins, antisense nucleic acids, ribosomes, PNA, siRNA, oligonucleotides, aptamer, and peptide aptamer and derivatives and fragments thereof.

In some embodiments, antagonists to isoforms of p63 and/or DNp63 isoforms useful in the methods of the present invention can be a nucleic acid-based inhibitor, nucleic acid construct, a peptide-based inhibitor or a small molecule inhibitor of p63 isoforms or DNp63 isoforms or a polynucleotide encoding the same. In some embodiments a nucleic-acid inhibitor may be a RNAi (RNA interference) agent, such as for example a siRNA molecule or an antisense construct.

For example in some embodiments, p63 protein isoforms or DNp63 isoforms can be inhibited by nucleic acid-based inhibitors such as siRNA molecules, for example as disclosed in the Examples such as siRNA molecules comprising the nucleotide sequence set forth in any one of SEQ ID NO:7, SEQ ID NO:20 or SEQ ID NO:21 or a fragment thereof.

Another embodiment provides an isolated inhibitory nucleic acid construct comprising a nucleic acid sequence which specifically hybridizes to a least a portion of the polynucleotide encoding isoforms of DNp63, wherein the nucleic acid construct inhibits the expression of DNp63 protein isoforms in tumor cells. Alternatively, inhibitory nucleic acid constructs may comprise of a nucleic acid sequences specific to at least a portion of a polynucleotide encoding one or more genes which regulate the expression of isoforms of p63 or isoforms of DNp63. Genes that regulate the expression of isoforms of p63 or isoforms of DNp63 comprise, for example, but not limited to, transcription factors, co-activators, activators, enhancers and cofactors of p63 isoforms and/or DNp63 isoforms.

A portion of the polynucleotide may include the coding region of the gene encoding TAp63 isoforms or DNp63 isoforms, for example SEQ ID NO: 5 and SEQ ID NO:7 respectively, and/or one or more regulatory regions of the gene known to one skilled in the art.

In some embodiments, the nucleic acid construct may be a siRNA molecule, for example a siRNA targeting the inhibition of a p63 isoform such as a DNp63 isoform, i.e. such as siRNA molecules comprising SEQ ID NO: 20 and/or SEQ NO: 21 or any other nucleotide sequence designed from SEQ ID NO:7 which is specific to DNp63 isoforms and results in knock down of DNp63 protein.

In some embodiments, the nucleic acid construct may have a nucleotide sequence having at least 85% identity to the nucleotide sequence set forth in any one of SEQ ID NO: 20 and/or SEQ ID NO:21 or a fragment thereof.

In other embodiments, the nucleotide sequence may have at least 85%, at least 90% identity, or at least 95% identity, to the nucleotide sequence set forth in any one of SEQ ID NO: 20 and/or SEQ ID NO:21 or a fragment thereof.

In another embodiment, antagonists to p63 and/or DNp63 can be protein-based agents or antagonists, for example, but not limited to, small peptide molecules, antibodies, chimeric antibodies, humanized antibodies, human antibodies, recombinant proteins. Protein-based antagonists can be a p73 isoform or TAp73 or DNp73 isoform decoy molecule or protein. For example, a p73 isoform or TAp73 isoform decoy protein can comprise a region of a functional and/or non-functional p73 or TAp73 or DNp73 protein or fragment thereof, that sequesters isoforms of p63, for example sequesters DNp63 isoforms.

In another embodiment, the antagonists to p63 isoforms and DNp63 isoforms can prevent them from forming a p63:p73 complex, or disrupt an already formed p63:p73 complex. In one such embodiment, a p63:p73 complex comprises isoforms of DNp63 and isoforms of TAp73. Antagonists of agents that disrupt a p63:p73 complex can be any agent such as but not limited to protein-based antagonists, small molecule, peptide, protein, antisense nucleic acid, ribosome, PNA, siRNA, oligonucleotide aptamer, and peptide aptamer.

Another aspect of the present invention relates to methods to produce pharmacological compositions for inhibiting the expression or activity of isoforms of DNp63, thereby reducing DNp63-mediated inhibition of p73 isoforms. In some embodiments, the present invention provides methods for treating an cancer in a subject comprising administering to the subject an antagonist or inhibitory agent of p63 isoforms and/or DNp63 isoforms as disclosed herein, for example but not limited to antibodies, antibody fragments, small molecules, peptides, proteins, antisense nucleic acids, ribosomes, PNAs, siRNAs, oligonucleotides, aptamers, peptide aptamers, with one or more pharmaceutically acceptable carriers, diluents and adjuvants.

Another aspect of the present invention relates to methods for increasing the sensitivity of tumor cells to p73/p63 targeting treatments, the method comprising administering to the tumor cell an effective amount of an agent that inhibits DNp63 isoforms in an effective amount to attenuate DNp63-mediated inhibition of p73 isoforms.

Another aspect of the present invention relates to methods to produce pharmacological compositions for increasing sensitivity of tumor cells to at least one p73/p63 targeting treatments, the composition comprising at least one antagonist or inhibitor agent of p63 isoforms or DNp63 isoforms as disclosed herein, for example an inhibitor agent or antagonist such as, but not limited to; an antibody, antibody fragment, small molecule, peptide, protein, antisense nucleic acid, ribosome, PNA, siRNA, oligonucleotide aptamer, and peptide aptamer, together with one or more pharmaceutically acceptable carriers, diluents and adjuvants.

In some embodiments, the methods as disclosed herein are useful in the prevention and/or treatment of cancers such as, but are not limited to, tumors selected from a group comprising of gastrointestinal cancer, gastric cancer, squamous cell carcinomas (SCC), head and neck cancer, lung cancer, non-small cell lung cancer (NSCLC) and small-cell lung cancer (SCLC), lymphoma, sarcoma, primary and metastic melanoma, thymoma, non-Hodgkin's lymphoma, Hodgkin's lymphoma, cancer of the nervous system, brain cancer, bone-marrow cancer, bone cancer, kidney cancer, uterine cancer, cervival cancer, colon cancer, retina cancer, skin cancer, bladder cancer, colon cancer, esophageal cancer, testicular cancer, cervical cancer, liver cancer, renal cancer, pancreatic cancer, genital-urinary cancer, gastrointestinal, gum cancer, tongue cancer, kidney cancer, nasopharynx cancer, stomach cancer, endometrial cancer and bowel tumor cell cancer, adrenocarcinomas such as prostate cancer, ovarian cancer, breast cancer, and pancreatic cancer. In particular, the cancer is breast cancer, for example the triple-negative subtype of breast cancer.

Another aspect of the present invention provides a kit for implementing the QRT-PCR or p63 isoforms such as DNp63 isoforms and p73 isoforms such as TAp73 or DNp73 isoforms. In some embodiments, the kit comprises at least one primer set designed to anneal to the cDNA of DNp63 isoforms and at least one primer set designed to anneal to the cDNA of p73 isoforms. Additionally, the kit can also optionally comprise a nucleic acid probe designed to anneal to the cDNA of p63, and a nucleic acid probe designed to anneal to the cDNA of p73 isoforms. In some embodiments, the kit can also optionally comprise products, reagents as well as positive and negative controls useful in carrying out QRT-PCR amplification, and instructions comprising methods for analysis of the amplification products.

In one embodiment, the kit for implementing the QRT-PCR enables an isoform-specific QRT-PCR, wherein the kit comprises at least one set of the primer pairs contained within the kit are designed to anneal to isoforms of DNp63 and at least one set of primer pairs are designed to anneal to isoforms of p73 such as TAp73 or DNp73 isoforms. Additionally, the kit may optionally also contain at least one probe is designed to anneal to DNp63 isoforms and at least one probe is designed to anneal to TAp73 isoforms.

In some embodiments, primer pairs useful in an isoform-specific kit are disclosed herein, for example but are not limited to SEQ ID NO:9 and SEQ ID NO:10 for forward and reverse primers, respectively for TAp73 isoforms and SEQ ID NO:11 and SEQ ID NO:12 for forward and reverse primers, respectively for DNp63 isoforms.

In another embodiment, primer pairs useful in a non-isoform specific kit are disclosed herein, for example can be, but are not limited to SEQ ID NO:13 and SEQ ID NO:14 for forward and reverse primers, respectively for p73 isoforms and SEQ ID NO:15 and SEQ ID NO:16 for forward and reverse primers, respectively for p63 isoforms. In some embodiments, kits can optionally comprise of nucleic acid probes selected from a group comprising SEQ ID NO:17 and SEQ ID NO:18.

Another aspect of the present invention provides methods for identifying agents, compounds or entities which inhibit p63, such as DNp63 or function as antagonists of p63 isoforms. In some particular embodiments, methods relating to selecting antagonists or inhibitors of DNp63 isoforms are disclosed. Methods to assay for potential antagonists or inhibitors are known to persons skilled in the art can be used to identify antagonists or inhibitors of p63 isoforms such as DNp63, but as an illustrative example only, such methods comprise contacting an isoform of p63 with a potential agent compound and analysis of the expression p73 isoform effector genes such as pro-apoptotic genes NOXA and PUMA. An increase in these effector genes identifies an agent inhibitor of p63 isoform or DNp63 isoform (see Example 2, FIG. 2A and FIG. 3). In alternative embodiments, the methods comprise contacting an isoform of p63 such as DNp63 with a potential agent or compound and analysis of the binding and/or interaction with p73 isoforms (see Example 3).

In one embodiment, a p73/p63 targeting treatment is used to treat cancer. In some embodiment the cancer is epithelial in origin, for example, the cancer is, but is not limited to; gastrointestinal cancer, prostate cancer, ovarian cancer, breast cancer, head and neck cancer, lung cancer, small-cell lung cancer, cancer of the nervous system, kidney cancer, retina cancer, skin cancer, liver cancer, pancreatic cancer, genital-urinary cancer and bladder cancer.

In a particular embodiment, the methods as disclosed herein provide methods to treat and prevent a subject from having a triple-negative subtype of breast cancer. Without being bound by theory, the triple-negative subtype of breast cancer lacks the expression of the progesterone receptor (PR) and estrogen receptor (ER) and also lacks Her-2 amplification. In accordance to the methods, a subject who is identified to have responsiveness to a p73/p63 targeting treatment by the methods as disclosed herein is administered a pharmaceutical composition comprising a p73/p63 targeting treatment. In some embodiments, administration of a pharmaceutical composition comprising a p73/p63 targeting treatment can be alone or at the same time, or before or after, the administration of a pharmaceutical composition comprising an anti-cancer therapy as disclosed herein or a pharmaceutical composition which comprises an agent that inhibits the expression or activity of p63 isoforms, for example a pharmaceutical compound comprising a compound which inhibits DNp63 isoforms.

In some embodiments, a subject identified to be responsiveness to a p73/p63 targeting treatment by the methods as disclosed herein, or expresses both DNp63 and p73 isoforms such as TAp73 or DNp73 is administered a pharmaceutical composition comprising a specific p63 isoform antagonist or agent inhibiting p63, such as an agent inhibiting DNp63. In some embodiments, such a pharmaceutical composition comprising an inhibitor agent of p63 isoform or a p63 antagonist can be administered alone or at the same time, before or after the administration of a pharmaceutical composition comprising an anti-cancer therapy.

In another embodiment, a pharmaceutical composition as disclosed herein can be administered alone or with one or more other therapeutic active agents. For example, in the treatment of cancer, the pharmaceutical composition can be administered substantially at the same time as, or subsequent to administration of an anti-cancer therapy, such as, for example, chemotherapy, radiotherapy, hormone therapy, thermal therapy, immunotherapy, surgical resection and alternative cancer therapies commonly known by persons of ordinary skill in the art. Such anti-cancer therapies can be administered prior to, during or after administration of the pharmaceutical composition as disclosed herein. In some embodiments, the anti-cancer therapy is administered once, or more than once to the subject.

It is contemplated that any methods or compositions described herein can be implemented with respect of any other methods or compositions.

Other objects, features and advantages will become apparent from the following detailed description. It should be understood, however, that the detailed description and specific examples, while indicating specific embodiments of the invention, are given by way of illustration only, since various changes and modifications within the spirit and scope if the invention will become apparent to those skilled in the art from this detailed description.

### BRIEF DESCRIPTION OF THE FIGURES

FIG. 1 shows p63 is expressed in a subset of primary breast carcinomas. Panel 1A shows overexpression of p63 in primary microdissected invasive breast carcinomas relative to specimen-matched normal epithelia. Ratio of tumor/normal p63 mRNA determined by real-time quantitative RT-PCR (QRT-PCR). Panel 1B shows nuclear p63 protein correlates with p63 mRNA expression, as assessed by immunohistochemistry of representative tumors from panel 1A exhibiting low and high expression of p63 mRNA (original magnification×100). Panel 1C shows expression of p63 mRNA and its correlation with ΔNp63α protein (panel 1D) in a subset of human breast carcinoma-derived cell lines; MCF-7, HCC-1937, MDA-MB-468 and T47D.

FIG. 2 shows endogenous p63 is required for survival of breast cancer cells and functions as a survival factor in breast cancer cells through repression of p73-dependent apoptosis. Panel 2A shows knockdown of endogenous p63 induced PUMA, NOXA and PARP-1 cleavage, as assessed by immunoblot of the indicated cells 72 hours following infection with lentiviral shRNA vectors targeting two distinct p63 sequences (p63si-1, p63si-2) or a non-specific sequence (si-NS). None of these effects are observed in MCF-7 cells, which do not express abundant p63. Panel 2B shows apoptosis is observed following p63 knockdown and is specific to cells with endogenous p63 expression. Annexin/PI staining of unfixed cells 72 hours following infection with the indicated lentiviral shRNA vectors. Little or no cell death is observed in MCF-7 cells. Panels 2C, 2D and 2E shows PARP cleavage, PUMA induction, and apoptosis induced by p63 knockdown are TAp73-dependent. Panel 2C shows pools of cells expressing a TAp73-targeted shRNA or the control vector were then infected with a p63-directed lentiviral shRNA or control, and lysates were harvested at 72 hours for immunoblot. Panel 2D shows apoptotic morphologic features are TAp73-dependent. Photomicrograph of representative fields of cells treated as in 2C, 72 hours following p63 knockdown. Panel 2E shows quantitation of apoptosis by Annexin/PI staining of cells treated as in panel 2C and harvested 72 hours following p63 knockdown.

FIG. 3 shows lentiviral shRNA-mediated knockdown of p63 induces pro-apoptotic genes and cell death. Panel 3A shows knockdown of endogenous ΔNp63 mRNA by lentiviral p63-directed shRNA (p63si). T47D cells underwent lentiviral infection; RNA was prepared at 48 hours and assayed by isoform-specific QRT-PCR, normalized to GAPDH levels. Similar results were obtained in HCC-1937 cells (not shown). Panel 3B shows specific induction of PUMA and NOXA but not other pro-apoptotic genes correlates with apoptosis following lentiviral p63 knockdown in HCC-1937 cells, and neither gene induction nor apoptosis is observed in p63-negative MCF-7 cells. Bottom left, photomicrographs taken at 96 hours post lentiviral shRNA infection. Right, QRT-PCR for the indicated genes. For 3A and 3B, error bars represent the mean of two experiments performed in duplicate.

FIG. 4 shows endogenous p63 is required for survival in breast cancer cells. Apoptosis was observed following endogenous p63 knockdown, as assessed by annexin V/PI staining of unfixed cells 72 hours following infection with the indicated lentiviral shRNA vectors. Percentages indicate apoptotic cells (annexin V-positive and/or PI-positive).

FIG. 5 shows PUMA induction and apoptosis following p63 knockdown are TAp73-dependent in T47D cells. Panel 5A shows cells expressing a TAp73-directed shRNA or control were subsequently infected with a p63-directed lentiviral shRNA and harvested at 72 hours for immunoblot. Panel 5B shows photomicrograph taken 96 hours post lentiviral infection as in a show that morphologic features of apoptosis correlate with PUMA induction and PARP cleavage

FIG. 6 shows TAp73 and ΔNp63 are co-expressed in a subset of triple-negative primary breast carcinomas. Panel 6A shows TAp73 is overexpressed in ER/PR-negative (ER(−)) tumors. Shown is QRT-PCR for TAp73 in 14 ER-positive and 23 ER/PR-negative primary breast carcinomas. Below, statistical significance analyzed using both a mean-value approach (ER-positive, 0.373±0.126, ER/PR-negative 1.381±0.303, p=0.0172; *P value by 2-way Students t-test) and a binning approach, whereby tumors exhibiting more than two fold the mean of the sample set were categorized as HIGH and the rest as LOW (p=0.0309; *P value by Fisher's Exact Test). Panel 6B shows expression of TAp73 is correlates with ΔNp63 overexpression (p=0.0107, Fisher's Exact Test) and with p53 mutation (p=0.0257, Fisher's Exact Test) in ER/PR-negative primary breast carcinomas (*P values, Fisher's Exact Test). ΔNp63 levels were determined by QRT-PCR, and p53 mutation by cDNA sequencing. Note TAp73/ΔNp63 co-expression are not observed in HER-2 overexpressing tumors (assessed by QRT-PCR).

FIG. 7 shows quantitative evaluation of TAp73 mRNA and protein in breast cancer cells. Panel 7A shows TAp73 protein level by IP/Immunoblot of lug protein lysate from the indicated human breast cancer-derived cell lines. Little or no ΔNp73 is detected based on transfected isoform size controls and on isoform-specific QRT-PCR (not shown). Panel 7B shows quantitation of TAp73 by isoform-specific QRT-PCR, normalized to GAPDH. Panel 7C shows the correlation of TAp73 mRNA and protein levels in breast cancer cells.

FIG. 8 shows TAp73 mediates cisplatin sensitivity in breast cancer cells expressing TAp73/ΔNp63. Panel 8A shows inhibition of TAp73 induced resistance specifically to cisplatin. Dose-response curves (MTT cell viability assay) of cells expressing the control vector or a TAp73-directed lentiviral shRNA, 5 days following treatment with cisplatin (Cis), doxorubicin (Dox), or paclitaxel (Tax). Little or no effect of TAp73 knockdown was observed in MCF-7 cells. Error bars show SD for three independent experiments. Panel 8B shows TAp73 mediates selective proapoptotic target gene induction in response to cisplatin. QRT-PCR analysis of the indicated genes in HCC-1937 cells shown in 8A, 6 hours following cisplatin treatment (at the IC₇₀ dose of 6.6 μM). Panel 8C shows TAp73 expression conveys specific cisplatin sensitivity to normal basal mammary epithelial cells. MCF-10A cells were infected with a retrovirus encoding TAp73β or the control vector, followed by quantitative dose-response analysis as shown in 8A. (p<0.01, 1-tailed students t-test) TAp73β increases sensitivity (i.e. decreased the IC₇₀) only for cisplatin.

FIG. 9 shows TAp73 mediates cisplatin sensitivity in breast cancer cells. Panel 9A shows the knockdown of endogenous TAp73 by lentiviral shRNA in the indicated cell lines, assessed by IP/immunoblot. Panel 9B shows IC₅₀ values determined by cell viability (MTT) assay 5 days following treatment of control or TAp73-directed shRNA-expressing cells with the indicated agents. Panel 9C shows stable retroviral expression of TAp73β in MCF-9A cells, assessed by immunoblot. Panel 9D shows TAp73β expression in mammary epithelial cells conveys specific sensitivity to cisplatin. IC₅₀ values determined as in 9B.

FIG. 10 shows c-Abl-dependent phosphorylation is induced specifically by cisplatin and is required for TAp73 activation and cisplatin sensitivity. Panel 10A shows TAp73 is tyrosine phosphorylated in response to cisplatin (Cis) but not doxorubicin (Dox). Immunoprecipitated p73 was probed for anti-phosphorylated tyrosine (p-tyr) by immunoblot 6 hours following control, cisplatin, or doxorubicin treatment (each at the IC₅₀ dose). The same blot was then stripped and re-probed for total p73 protein. HCC-1937 cells express TAp73α, while MDA-MB-468 cells express both TAp73α and TAp73β. Panel 10B shows c-Abl-dependent phosphorylation of TAp73 following cisplatin treatment. Cells were pretreated with STI-571 (1 μM, 2 hours) or vehicle control as indicated, then were treated with cisplatin and analyzed as in 10A. Panel 10C shows p73-dependent pro-apoptotic transcription requires c-Abl-mediated phosphorylation. Cells were pre-treated with STI-571 (also referred to herein as Imatinib or Ima) (1 μM, 2 hours) and/or treated with cisplatin for (3×IC₅₀ dose, 6 hours) as indicated, and mRNA was analyzed by QRT-PCR. Panel 10D shows c-Abl-dependent phosphorylation is important for cisplatin sensitivity. Cells were pre-treated with STI-571 as in c, then treated with cisplatin (IC₅₀ dose) and analyzed for viability by MTT at 5 days.

FIG. 11 shows cisplatin induces dissociation of the DNp63α/TAp73 complex. Panel 11A shows quantitative binding of TAp73 to DNp63α in HCC-1937 cells and dissociation following cisplatin treatment. Left, IPs of control or cisplatin-treated cultures (IC₇₀ for 6 hours); right, corresponding post-IP supernatants (Sup). IP for either p63 or p73 resulted in complete immunodepletion of TAp73 (lanes 11 and 12). Following cisplatin treatment, less TAp73 was associated with DNp63α (lanes 3 and 7), resulting in detectable “free” TAp73 in the depleted post-IP supernatant (lanes 11 and 15). Note the absence of change in DNp63α or TAp73 protein levels following cisplatin treatment (lanes 2, 6, 10, and 14). Controls demonstrated these antibodies to be non-cross-reactive (data not shown). Panel 11B shows MDA-MB-468 cells showed quantitative DNp63α/TAp73 binding similar to that of HCC-1937 cells and partial dissociation following cisplatin treatment. Cells were treated as in 11A. Left, IP product; right, post-IP supernatant. Note the decrease in TAp73 associated with DNp63α following cisplatin treatment (lanes 3 and 7), despite no change in DNp63α or TAp73 protein levels (lanes 10 and 14). HCC-1937 cells expressed TAp73α (12A), while MDA-MB-468 cells expressed both TAp73α and TAp73β (11B).

FIG. 12 shows TAp73 phosphorylation at Y99 is required for cisplatin-induced DNp63α/TAp73 dissociation and cell death in MCF-10A cells. Panel 12A shows wild-type or Y99F TAp73α were expressed in MCF-10A cells via retrovirus. Lysates from either cisplatin-treated or untreated cells were halved and subjected to IP for either p63 or p73, followed by immunoblots as shown. Wild-type TAp73α was tyrosine phosphorylated and dissociated from DNp63α following cisplatin treatment (1 μM, 6 hours), while Y99F TAp73α remained unphosphorylated and bound to DNp63α. Note there was no change in the total level of retroviral TAp73α or endogenous dNp63α following cisplatin treatment. Panel 12B shows TAp73 Y99 phosphorylation was required to convey cisplatin sensitivity. MCF-10A cells described in 12A were treated with cisplatin (1 μM) for 5 days, and cell viability was assessed by MTT. Error bars show SD for 3 independent experiments.

FIG. 13 shows Imatinib (also referred to as “STI571” herein) treatment blocks DNp63α/TAp73 dissociation, TAp73-dependent transcription, and cell death induced by cisplatin. Panel 13A shows Imatinib attenuated dissociation of DNp63α and TAp73. In left blots (control), cells were treated with imatinib (1 μM for 8 hours) or vehicle control; in right blots, cells were pretreated with imatinib (1 μM for 2 hours) or vehicle control, then treated with cisplatin (IC₇₀ for 6 hours) prior to IP for p63 or p73. Dissociation of TAp73 and DNp63α following cisplatin treatment (compare lanes 3 and 11) was attenuated by imatinib treatment (lane 15). Panels 13B and 13C show TAp73-dependent proapoptotic transcription required c-Abl-mediated phosphorylation.

### DETAILED DESCRIPTION

The present invention relates to methods to determine the level of sensitivity or resistance of tumor cells to p73/p63 targeting treatments, such as chemotherapeutic agents such as cisplatin, and thus the responsiveness of subjects with tumors to such agents. The present invention is based on discovery that a tumor cell expressing or having the activity of both a specific isoform of p63, such as DNp63 isoform, and a specific isoform of p73, such as TAp73 or DNp73, such a the tumor cell is responsive to p73/p63 targeting treatments, such as chemotherapeutic agents such as cisplatin or derivatives thereof.

Accordingly, one aspect of the present invention provides methods to detect the expression and/or activity of p63 isoforms, such as DNp63 isoform, and p73 isoforms, such as TAp73 or DNp73 in a biological sample from a subject, and if the biological sample is determined to express and/or have active p63 isoforms such as DNp63 isoform, and express and/or have active p73 isoforms such as TAp73 or DNp73, the subject is identified as being likely to be responsive to a p73/p63 targeting treatment such as cisplatin or derivatives thereof.

One aspect of the present invention relates to a method to determine the likelihood of a p73/p63 targeting treatment being effective in a subject affected with, or at risk of developing cancer. In one embodiment the cancer is breast cancer of the triple-negative subtype. In one embodiment, the method comprises detecting the expression and/or activity of both the p63 gene and the p73 gene in the subject. As disclosed herein, p63 and p73 are both members of the p53 family of transcription factors. In one embodiment, the method to determine the likelihood of a p73/p63 targeting treatment being effective in a subject affected with, or at risk of developing cancer comprises determining the expression or activity of the p63 is isoform DNp63a isoform, and the expression or activity of the p73 is isoform TAp73 or DNp73. In particular, the presence of both a DNp63 isoform, and a p73 isoform such as TAp73 or identifies that a p73/p63 targeting treatment is likely to be effective in the subject.

In one embodiment, a p73/p63 targeting treatment is likely to be effective in the subject if a biological subject obtained from the subject comprises the expression level or activity level of DNp63 at a higher molar ratio than the expression or activity of p73 isoform such as TAp73 or DNp73. For example, if the level of a DNp63 isoform is at least 1.2 fold, or at least 1.5 fold or greater, for example greater than 2.0-fold, than the level of a p73 isoform, such as TAp73 or DNp73.

In another embodiment, the methods as disclosed herein relate to a method to identify if a cancer is unresponsive to a p73/p63 targeting treatment such as cisplatin, where a cancer unresponsive is identified as a cancer cell that does not express or have the activity of DNp63 isoforms. In such an embodiment, the method comprises measuring the expression and/or activity of at least one isoform of DNp63 in at least one cancer cell, wherein the absence of expression and/or activity of a DNp63 isoforms identifies the cancer as being more likely to be unresponsive to cisplatin or a derivative thereof, as compared to a cancer wherein the expression or the activity of DNp63 is detected.

In another embodiment, a cancer cell is identified as being unresponsive to a p73/p63 targeting treatment if the level of expression of DNp63 in the cancer cell below a level DNp63 of a reference level, for example where a reference level is, but not limited to a level of DNp63 in a cancer cell responsive to a p73/p63 targeting treatment such as cisplatin.

Accordingly, another aspect of the present invention provides a method to identify a subject with responsiveness to a p73/p63 targeting treatment, such as for example a cancer treatment such as a platinum based cancer agent, for example but not limited to cisplatin or derivatives thereof.

One aspect of the present invention relates to a method for identifying a subject responsive to a p73/p63 targeting treatment, the method comprising determining the presence of expression and/or activity of both p73 and p63 indicates that a p73/p63 targeting treatment will likely be effective in the subject. Subjects identified to be responsive to p73/p63 targeting treatment can then be treated with a p73/p63 targeting treatment.

In some embodiments, a p73/p63 targeting treatment useful in the methods as disclosed herein is a chemotherapeutic agent such as a platinum-based chemotherapeutic agent, for example, but not limited to, cisplatin, synthetic cisplatin, cisplatin compounds, cisplatin metabolites, derivatives or analogues thereof.

In an alternative embodiment, subjects identified to be responsive to p73/p63 targeting treatment can be administered an inhibitor agent of p63, such as an inhibitor agent or antagonist of DNp63 isoforms.

In some embodiments, cancers useful to be treated in the methods as disclosed herein, include, for example but are not limited to cancers comprising those of epithelial origin, including, but are not limited to, gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer. In one embodiment, the cancer is non-small cell lung cancer. In another embodiment, the cancer is triple-negative subtype of cancer, which lacks the expression of the progesterone receptor (PR), the estrogen receptor (ER) and also lacks Her-2 amplification.

Tumor cell types can also be selected from a group comprising of gastrointestinal cancer, gastric cancer, squamous cell carcinomas (SCC), head and neck cancer, lung cancer, non-small cell lung cancer (NSCLC) and small-cell lung cancer (SCLC), lymphoma, sarcoma, primary and metastic melanoma, thymoma, non-Hodgkin's lymphoma, Hodgkin's lymphoma, cancer of the nervous system, brain cancer, bone-marrow cancer, bone cancer, kidney cancer, uterine cancer, cervival cancer, colon cancer, retina cancer, skin cancer, bladder cancer, colon cancer, esophageal cancer, testicular cancer, cervical cancer, liver cancer, renal cancer, pancreatic cancer, genital-urinary cancer, gastrointestinal, gum cancer, tongue cancer, kidney cancer, nasopharynx cancer, stomach cancer, endometrial cancer and bowel tumor cell cancer, adrenocarcinomas such as prostate cancer, ovarian cancer, breast cancer, and pancreatic cancer.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term “p73/p63 targeting treatment” as used herein refers to any treatment which mediates some, all or part of its biological function, through acting directly or indirectly on the gene product or polynucleotide of isoforms of p73 and/or isoforms of p63. A p73/p63 targeting treatment can directly or indirectly activate isoforms of p73 and/or directly or indirectly inactivate or inhibit isoforms of p63. In some embodiments, a p73/p63 targeting treatment activates p73 isoforms by attenuating p63 isoform-mediated sequestering of p73 isoforms, for example a p73/p63 targeting treatment disrupts the p63:p73 complex to release TAp73 isoforms so that TAp73 can induce pro-apoptotic genes such as NOXA and PUMA. In some embodiments, a p73/p63 targeting treatment is a specific p63 isoform antagonist or inhibitor agent, for example an agent that inhibits p63, but for example but not limited to an agent that inhibits p63 or inhibit p63 isoforms in a DNp63:TAp73 complex such that TAp73 is released.

Exemplarily examples of p73/p63 targeting treatments are for example but not limited to, chemotherapeutic agents, such as platinum based chemotherapeutic agents. Examples include, but are not limited to cisplatin (cis-diaminedichloroplatinuim (II), cis-DDP, CDDP), cisplatin compounds, cisplatin metabolites, derivatives or analogues thereof having a skeleton similar to cisplatin. Analogues of cisplatin, for example include, but are not limited to, carboplaitin (cis-diamine[1,1-cyclobutnaedicarboxylate(2-)-O,O′-platinum(II)) and oxaliplatin (cis-L-diaminocyclohexane oxalotoplatinum (II). Cisplatin derivatives include, for example but not limited to, those set forth in U.S. Patent Application No: US2006/0142593, which is incorporated herein in its entirety by reference.

As used herein, the terms “effective” and “effectiveness” includes both pharmacological effectiveness and physiological safety. “Pharmacological effectiveness” refers to the ability of the treatment to result in a desired biological effect in the subject. “Physiological safety” refers to the level of toxicity, or other adverse physiological effects at the cellular, organ and/or organism level (often referred to as side-effects) resulting from administration of the treatment, “less effective” means that the treatment results in a therapeutically significant lower level of pharmacological effectiveness and/or a therapeutically greater level of adverse physiological effects.

The term “lack of effectiveness”, “non-responsiveness”, “refractory” or “unresponsiveness” are used interchangeably herein, and refer to the inability of an agent or treatment to result in a desired biological effect in the subject.

The term “effective amount” includes within its meaning a sufficient amount of a pharmacological composition to provide the desired effect. The exact amount required will vary depending on factors such as the level of expression of isoforms of p63 and/or isoforms of p73 proteins in the absence of the pharmaceutical composition, the type of tumor to be treated, the severity of the tumor, the drug resistance level of the tumor, the species being treated, the age and general condition of the subject, the particular treatment being used, such as an antagonist to isoforms of p63 and/or a particular p73/p63 targeting treatment being administered, the mode of administration and so forth. Thus, it is not possible to specify the exact “effective amount”. However, for any given case, an appropriate “effective amount” may be determined by one of ordinary skill in the art using only routine experimentation. As used herein, the effective amount is the amount of an agent or treatment to reduce a symptom of the disease, for example, but not limited to, to reduce the size of a tumor, for example to reduce the size by about 10%, to attenuate the growth rate of the tumor, for example to reduce the rate at which a tumor grows by 10%. For example, an effective amount using the methods as disclosed herein would be considered as the amount sufficient to reduce a symptom of the cancer, for example at least one symptom of a cancer or malignancy by at least 10%. Further, an effective amount as used herein would also include an amount sufficient to prevent or delay the development of a symptom of the disease, alter the course of a symptom disease (for example but not limited to, slow the progression of a symptom of the disease), or reverse a symptom of the disease.

As used herein, the term “treating” includes reducing or alleviating at least one adverse effect or symptom of a condition, disease or disorder associated with cancer. As used herein, the term treating is used to refer to the reduction of a symptom and/or a biochemical marker of cancer, for example a reduction in at least one biochemical marker of cancer by at least 10%. For example but are not limited to, a reduction in a biochemical marker of cancer, for example a reduction in, as an illustrative example only, at least one of the following biomarkers; CD44, telomerase, TGF-α, TGF-β, erbB-2, erbB-3, MUC1, MUC2, CK20, PSA, CA125, FOBT, by 10%, or a reduction in the rate of proliferation of the cancer cells by 10%, would be considered effective treatments by the methods as disclosed herein. As alternative examples, a reduction in a symptom of cancer, for example, a slowing of the rate of growth of the cancer by 10% or a cessation of the increase in tumor size, or a reduction in the size of a tumor by 10% or a reduction in the tumor spread (i.e. tumor metastasis) by 10% would also be considered as affective treatments by the methods as disclosed herein.

The term “p73” and “TAp73” are used interchangeably herein and refers collectively to isoforms of the tumor protein p73 also known as TP73 that contains the N-terminal tranactivation domain. p73 refers to the gene encoding isoforms of p73 protein, which can be expressed by as multiple isoforms resulting from activation of multiple promoters and/or from alternative mRNA splicing. Identification of the gene can be by the following IDs: HGNC ID:12003 Accession No. ID:AB055065; RefSeq ID: NM_{—}005427 or www.ncbi.nlm.nih.gov/entrez/query.fcgi?db=gene&cmd=Retrieve&dopt=full_report&list_uids=7161

“TAp73a” or “TAp73α” and “TAp73β” as used herein refers to isoforms of TAp73 as a result of differential mRNA splicing to generate different C-terminal variants, the TAp73α is Refseq IDL NM-005427 (SEQ ID NO:1) or NP_{—}005418 (SEQ ID NO:2).

The term “DNp73” or “ΔNp73” are used interchangeably herein and refers to a delta-Np73 isoform, an isoform of the tumor protein p73 which lacks the N-terminal transactivation domain, for example RefSeq ID: AB_{—}055065, which corresponds to SEQ ID NO:2 or Accession number BAB87244 (SEQ ID NO:4).

The terms “p63” and “TAp63” are used interchangeably herein and refers collectively to isoforms of the tumor protein 63 (TP63), also known in the art as; tumor protein p73-like (TP73L or TP73-like or TPp63). The gene p63 is also known as the “p63 locus” and by alternative aliases comprising; B(p51A), B(p51B), EEC3, KET, LMS, OFC8, RHS, SHFM4, TP63, p51, p63, p73H, p73L. p63 refers to the gene encoding isoforms of p63 protein, for example RefSeq ID:AF075430 or www.ncbi.nlm.nih.gov/entrez/query.fcgi?itool=nucleotide_brief&db=nucleotide&cmd=Display&dop t=nucleotide_gene&from_uid=31543817.

The term “TAp63a” or “TAp63α” used interchangeably herein refer to isoforms of TAp63 as a result of differential mRNA splicing to generate different C-terminal variants, and for reference purposes only, refers to RefSeq ID: AF075430 (SEQ ID NO:5) or AAC62635 (SEQ ID NO:6).

The term “DNp63” or “ΔNp63” are used interchangeably herein and refer to a delta-Np63 isoform, an isoform of p63 which that lacks the N-terminal transactivation domain, for example, Accession No. AF_{—}075431.

The term “DNp63a” or “DNp63α” used interchangeably herein refers to isoforms of TAp63 alpha (TAp63α) that lacks the N-terminal transactivation domain as a result of differential mRNA splicing. DNp63a and DNp63 are used interchangeably herein, and for reference purposes only, correspond to RefSeq ID: AF_{—}075431 (SEQ ID NO:7) and AAC62636 (SEQ ID NO:8).

The terms “isoform”, or “isoforms” refer to one specific form of protein gene in a population, the specific form differing from other forms of the same protein in the sequence of at least one, and frequently more than one, amino acids. Isoforms are proteins produced from the same gene, due to, for example but not limited to, transcription from different promoters, alternative splicing or differential mRNA splicing.

The term “p63 isoforms” or “isoforms of p63” includes all isoforms of the p63 gene including all DNp63 isoforms.

The term “specific p63 isoforms” encompasses all p63 isoforms, but more preferably DNp63 isoforms.

The term “p73 isoforms” or “isoforms of p73” includes all isoforms of the p73 gene.

The term “polynucleotide” as used herein, refers to single- or double-stranded polymer of deoxyribonucleotide, ribonucleotide bases or known analogies of natural nucleotides, or mixtures thereof. The term includes reference to the specified sequence as well as to the sequence complementary thereto, unless otherwise indicated.

The term “polypeptide” means a polymer made up of amino acids linked together by peptide bonds. The terms “polypeptide” and “protein” are used interchangeably herein, although for the purposes for the present invention, a polypeptide may constitute a portion or the full length protein.

The term “expression” as used herein refers to interchangeably to the expression of a polypeptide or protein and expression of a polynucleotide or gene. Expression of a polynucleotide may be determined, for example, by measuring the production of messenger RNA (mRNA) transcript levels. Expression of a protein or polypeptide may be determined, for example, by immunoassay using an antibody(ies) that bind with the polypeptide.

The term “endogenously expressed” or “endogenous expression” as used herein, refers to the expression of a gene product at normal levels and under normal regulation for that cell type.

The term “co-expression” refers to the expression of more than one polypeptide or protein within the same tissue sample. In a preferred embodiment, the co-expression refers to expression of more than one polypeptide or protein within the same cell.

In the context of this specification, the term “activity” as it pertains to a protein, polypeptide or polynucleotide means any cellular function, action, effect of influence exerted by the protein, polypeptide or polynucleotide, either by nucleic acid sequence or fragment thereof, or by the protein or polypeptide itself or any fragment thereof.

The term “real-time quantitative RT-PCR” or “quantitative RT-PCR” or “QRT-PCR” are used interchangeably herein, refers to reverse transcription (RT) polymerase chain reaction (PCR) which enables detection of gene transcription. The method is known to those ordinary skilled in the art and comprises of the reverse transcription and amplification of messenger RNA (mRNA) species to cDNA, which is further amplified by the PCR reaction. QRT-PCR enables a one skilled in the art to quantitatively measure the level of gene transcription from the test gene in a particular biological sample.

The term “multiplex” as used herein refers to the testing and/or the assessment of more than one gene within the same reaction sample.

The term “primer”, as used herein, refers to an oligonucleotide which is capable of acting as a point of initiation of polynucleotide synthesis along a complementary strand when placed under conditions in which synthesis of a primer extension product which is complementary to a polynucleotide is catalyzed. Such conditions include the presence of four different nucleotide triphosphates or nucleoside analogs and one or more agents for polymerization such as DNA polymerase and/or reverse transcriptase, in an appropriate buffer (“buffer” includes substituents which are cofactors, or which affect pH, ionic strength, etc.), and at a suitable temperature, A primer must be sufficiently long to prime the synthesis of extension products in the presence of an agent for polymerase. A typical primer contains at least about 5 nucleotides in length of a sequence substantially complementary to the target sequence, but somewhat longer primers are preferred. Usually primers contain about 15-26 nucleotides, but longer primers may also be employed.

A primer will always contain a sequence substantially complementary to the target sequence which is the specific sequence to be amplified to which it can anneal. A primer can optionally, also comprise a promoter sequence. The term “promoter sequence” defines a single strand of a nucleic acid sequence that is specifically recognized by an RNA polymerase that binds to a recognized sequence and initiates the process of transcription by which an RNA transcript is produced. In principle, any promoter sequence may be employed for which there is a known and available polymerase that is capable of recognizing the initiation sequence. Known and useful promoters are those that are recognized by certain bacteriophage polymerases, such as bacteriophage T3, T7 or SP6.

In the context of this invention, the term “probe” refers to a molecule which can detectably distinguish between target molecules differing in structure. Detection can be accomplished in a variety of different ways depending on the type of probe used and the type of target molecule, thus, for example, detection may be based on discrimination of activity levels of the target molecule, but preferably is based on detection of specific binding. Examples of such specific binding include antibody binding and nucleic acid probe hybridization. Thus, for example, probes can include enzyme substrates, antibodies and antibody fragments, and preferably nucleic acid hybridization probes.

The term “amplify” is used in the broad sense to mean creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases.

A “microarray” is a linear or two-dimensional array of preferably discrete regions, each having a defined area, formed on the surface of a solid support. The density of the discrete regions on a microarray is determined by the total numbers of target species to be detected on the surface of a single solid phase support, preferably at least about 50/cm², more preferably at least about 100/cm², even more preferably at least about 500/cm², and still more preferably at least about 1,000/cm². As used herein, a “DNA microarray” is an array of oligonucleotide primers placed on a chip or other surfaces used to amplify or clone target polynucleotides, where polynucleotides are the target species. A “protein array” or “protein microarray” is an array of proteins or polynucleotide places on a chip or other surfaces used to bind target protein species. Since the position of each particular group of primers in the array is known, the identities of the target polynucleotides can be determined based on their binding to a particular position in the microarray.

The term “label” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

The term “cancer”, as used herein refers to a cellular proliferative disease in a human or animal subject.

The term “tumor” or “tumor cell” used interchangeably herein refers to the tissue mass or tissue type or cell type that is undergoing uncontrolled proliferation.

The term “Triple-negative subtype” used herein refers to any subtype of cancer, particularly breast cancer, which lacks the expression of the progesterone receptor (PR), lacks the estrogen receptor (ER) and also lacks Her-2 amplification.

As used herein, a “biological sample” or “tissue sample” refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, a biological sample is from a resection, bronchoscopic biopsy, or core needle biopsy of a primary, secondary or metastatic tumor, or a cellblock from pleural fluid. In addition, fine needle aspirate biological samples are also useful. In some embodiments, a biological sample is primary ascite cells. Samples can be optionally paraffin-embedded, frozen or subjected to other tissue preservation methods. The term “biological sample” as used herein may mean a sample of biological tissue or fluid that comprises nucleic acids. Such samples include, but are not limited to, tissue isolated from animals. Biological samples may also include sections of tissues such as biopsy and autopsy samples, frozen sections taken for histological purposes, blood, plasma, serum, sputum, stool, tears, mucus, hair, and skin. Biological samples also include explants and primary and/or transformed cell cultures derived from patient tissues. A biological sample may be provided by removing a sample of cells from an animal, but can also be accomplished by using previously isolated cells (e.g., isolated by another person, at another time, and/or for another purpose), or by performing the methods of the invention in vivo. Archival tissues, such as those having treatment or outcome history may also be used. As used herein, the term “biological sample” also refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g. buccal scrapes), whole blood, plasma, serum, urine, saliva, cell culture, or cerebrospinal fluid. Biological samples also include tissue biopsies, cell culture. A biological sample or tissue sample can refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the sample is from a resection, bronchoscopic biopsy, or core needle biopsy of a primary or metastatic tumor, or a cellblock from pleural fluid. In addition, fine needle aspirate samples are used. Samples may be either paraffin-embedded or frozen tissue. The sample can be obtained by removing a sample of cells from a subject, but can also be accomplished by using previously isolated cells (e.g. isolated by another person), or by performing the methods of the invention in vivo. Biological sample also refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the biological samples can be prepared, for example biological samples may be fresh, fixed, frozen, or embedded in paraffin.

The term ‘malignancy’ and ‘cancer’ are used interchangeably herein, refers to diseases that are characterized by uncontrolled, abnormal growth of cells. Cancer cells can spread locally or through the bloodstream and lymphatic system to other parts of the body. The term “malignancy” or “cancer” are used interchangeably herein and refers to any disease of an organ or tissue in mammals characterized by poorly controlled or uncontrolled multiplication of normal or abnormal cells in that tissue and its effect on the body as a whole. Cancer diseases within the scope of the definition comprise benign neoplasms, dysplasias, hyperplasias as well as neoplasms showing metastatic growth or any other transformations like e.g. leukoplakias which often precede a breakout of cancer.

The term “tumor” or “tumor cell” are used interchangeably herein, refers to the tissue mass or tissue type of cell that is undergoing abnormal proliferation.

The term “tissue” is intended to include intact cells, blood, blood preparations such as plasma and serum, bones, joints, muscles, smooth muscles, and organs.

The terms “patient”, “subject” and “individual” are used interchangeably herein, and refer to an animal, particularly a human, to whom treatment including prophylaxic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model.

The term “nucleic acid” or “oligonucleotide” or “polynucleotide” used herein can mean at least two nucleotides covalently linked together. As will be appreciated by those in the art, the depiction of a single strand also defines the sequence of the complementary strand. Thus, a nucleic acid also encompasses the complementary strand of a depicted single strand. As will also be appreciated by those in the art, many variants of a nucleic acid can be used for the same purpose as a given nucleic acid. Thus, a nucleic acid also encompasses substantially identical nucleic acids and complements thereof. As will also be appreciated by those in the art, a single strand provides a probe for a probe that can hybridize to the target sequence under stringent hybridization conditions. Thus, a nucleic acid also encompasses a probe that hybridizes under stringent hybridization conditions.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994).

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

A nucleic acid will generally contain phosphodiester bonds, although nucleic acid analogs can be included that can have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog can be located for example at the 5′-end and/or the 3′-end of the nucleic acid molecule. Representative examples of nucleotide analogs can be selected from sugar- or backbone-modified ribonucleotides. It should be noted, however, that also nucleobase-modified ribonucleotides, i.e. ribonucleotides, containing a non naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7 deaza-adenosine; O— and N— alkylated nucleotides, e.g. N6-methyl adenosine are suitable. The 2′ OH— group can be replaced by a group selected from H. OR, R. halo, SH, SR, NH₂, NHR, NR₂ or CN, wherein R is C-C6 alkyl, alkenyl or alkynyl and halo is F. Cl, Br or I. Modifications of the ribose-phosphate backbone can be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs can be made.

The term “gene” used herein can be a genomic gene comprising transcriptional and/or translational regulatory sequences and/or a coding region and/or non-translated sequences (e.g., introns, 5′- and 3′-untranslated sequences and regulatory sequences). The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene can also be an amplified nucleic acid molecule produced in vitro comprising all or a part of the coding region and/or 5′- or 3′-untranslated sequences linked thereto.

The term “target” as used herein may mean a polynucleotide that may be bound by one or more probes under stringent hybridization conditions.

The term “agent” refers to any entity which is normally absent or not present at the levels being administered, in the cell. Agent may be selected from a group comprising; chemicals; small molecules; nucleic acid sequences; nucleic acid analogues; proteins; peptides; aptamers; antibodies; or fragments thereof. A nucleic acid sequence may be RNA or DNA, and may be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), etc. Such nucleic acid sequences include, for example, but not limited to, nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. The agent may be applied to the media, where it contacts the cell and induces its effects. Alternatively, the agent may be intracellular within the cell as a result of introduction of the nucleic acid sequence into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

The term “antagonist” refers to any agent or entity capable of inhibiting the expression or activity of a protein, polypeptide portion thereof, or polynucleotide. Thus, the antagonist may operate to prevent transcription, translation, post-transcriptional or post-translational processing or otherwise inhibit the activity of the protein, polypeptide or polynucleotide in any way, via either direct of indirect action. The antagonist may for example be a nucleic acid, peptide, or any other suitable chemical compound or molecule or any combination of these. Additionally, it will be understood that in indirectly impairing the activity of a protein, polypeptide of polynucleotide, the antagonist may affect the activity of the cellular molecules which may in turn act as regulators or the protein, polypeptide or polynucleotide itself. Similarly, the antagonist may affect the activity of molecules which are themselves subject to the regulation or modulation by the protein, polypeptide of polynucleotide. The term “antagonist of p63 isoforms” and “antagonist of DNp63 isoforms” as used interchangeably herein.

The term “inhibiting” as used herein as it pertains to the expression or activity of the protein or polypeptide of p63 isoforms or DNp63 isoforms does not necessarily mean complete inhibition of expression and/or activity. Rather, expression or activity of the protein, polypeptide or polynucleotide is inhibited to an extent, and/or for a time, sufficient to produce the desired effect.

The term “specific” when used in relation to the nucleic acid sequence of an inhibitory nucleic acid construct of the invention means substantially specific, but not necessarily exclusively so. For example, the nucleotide sequence of an inhibitory nucleic acid agent according to the methods as disclosed herein can display less than 100% sequence identity with a particular p63 polynucleotide isoform or DNp63 isoform and retain specificity thereto. The term “specific” when used in relation to a protein or polypeptide or an inhibitory protein or polypeptide of the invention means substantially specific, but not necessarily exclusively so. For example, an inhibitory protein or polypeptide according to the present invention may recognize one or more particular isoforms of the p63 protein and retain specificity thereto.

The term “entity” refers to any structural molecule or combination of molecules.

The term “RNAi” as used herein refers to RNA interference (RNAi) a RNA-based molecule that inhibits gene expression. RNAi refers to a means of selective post-transcriptional gene silencing by destruction of specific mRNA by small interfering RNA molecules (siRNA). The siRNA is typically generated by cleavage of double stranded RNA, where one strand is identical to the message to be inactivated.

The term “shRNA” as used herein refers to short hairpin RNA which functions as RNAi and/or siRNA species but differs in that shRNAi species are double stranded hairpin-like structure for increased stability.

As used herein, “gene silencing” or “gene silenced” in reference to an activity of n RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but are not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

As used herein an “siRNA” refers to a nucleic acid that forms a double stranded RNA, which double stranded RNA has the ability to reduce or inhibit expression of a gene or target gene when the siRNA is present or expressed in the same cell as the target gene, for example where a target gene is P63 isoforms, such as DNp63 isoforms. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The term “antibody” is meant to be an immunoglobulin protein that is capable of binding an antigen. Antibody as used herein is meant to include antibody fragments, e.g. F(ab′)₂, Fab′, Fab, capable of binding the antigen or antigenic fragment of interest. An antibody antagonist of p63 isoforms or DNp63 isoforms is an antibody binding to the antigen inhibits the activity of isoforms of p63 or isoforms of DNp63.

The term “humanized antibody” is used herein to describe complete antibody molecules, i.e. composed of two complete light chains and two complete heavy chains, as well as antibodies consisting only of antibody fragments, e.g. Fab, Fab′, F(ab′)₂, and Fv, wherein the CDRs are derived from a non-human source and the remaining portion of the Ig molecule or fragment thereof is derived from a human antibody, preferably produced from a nucleic acid sequence encoding a human antibody.

The terms “human antibody” and “humanized antibody” are used herein to describe an antibody of which all portions of the antibody molecule are derived from a nucleic acid sequence encoding a human antibody. Such human antibodies are most desirable for use in antibody therapies, as such antibodies would elicit little or no immune response in the human subject.

The term “chimeric antibody” is used herein to describe an antibody molecule as well as antibody fragments, as described above in the definition of the term “humanized antibody.” The term “chimeric antibody” encompasses humanized antibodies. Chimeric antibodies have at least one portion of a heavy or light chain amino acid sequence derived from a first mammalian species and another portion of the heavy or light chain amino acid sequence derived from a second, different mammalian species. In some embodiments, a variable region is derived from a non-human mammalian species and the constant region is derived from a human species. Specifically, the chimeric antibody is preferably produced from a 9 nucleotide sequence from a non-human mammal encoding a variable region and a nucleotide sequence from a human encoding a constant region of an antibody.

The term “drug”, “agent” or “compound” as used herein refers to a chemical entity or biological product, or combination of chemical entities or biological products, administered to a person to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The term “anti-cancer agent” or “anti-cancer drug” as used herein refers to any agent, compound or entity that would be capably of negatively affecting the cancer in the subject, for example killing cancer cells, inducing apoptosis in cancer cells, reducing the growth rate of cancer cells, reducing the number of mestatic cells, reducing tumor size, inhibiting tumor growth, reducing blood supply to a tumor or cancer cells, promoting an immune response against cancer cells or a tumor, preventing or inhibiting the progression of cancer, or increasing the lifespan of the subject with cancer. An anti-cancer therapy encompasses any immunotherapy or biological agent (biotherapy), chemotherapy agents, and radiotherapy agents. The combination of chemotherapy with biological therapy is known in the art as biochemotherapy.

The term “cisplatin” can include a “mimetic” of cisplatin or a derivative thereof, which includes compounds which may not be structurally similar to cisplatin but mimic the therapeutic activity or therapeutic mechanism of cisplatin or structurally similar cisplatin compound in vitro and in vivo.

Compositions or methods “comprising” one or more recited elements may include other elements not specifically recited. For example, a composition that comprises a fibril component peptide encompasses both the isolated peptide and the peptide as a component of a larger polypeptide sequence. By way of further example, a composition that comprises elements A and B also encompasses a composition consisting of A, B and C. The terms “comprising” means “including principally, but not necessary solely”. Furthermore, variation of the word “comprising”, such as “comprise” and “comprises”, have correspondingly varied meanings. The term “consisting essentially” means “including principally, but not necessary solely at least one”, and as such, is intended to mean a “selection of one or more, and in any combination.” In the context of the specification, the term “comprising” means “including principally, but not necessary solely”. Furthermore, variation of the word “comprising”, such as “comprise” and “comprises”, have correspondingly varied meanings.

The use of the word “a” or “an” when used in conjunction with the term “comprising” in the claims and/or the specification may mean “one” but is also consistent with the meaning of “one or more”, “at least one” and “one or more than one.”

Other than in the operating examples, or where otherwise indicated, all numbers expressing quantities of ingredients or reaction conditions used herein should be understood as modified in all instances by the term “about.” The term “about” when used in connection with percentages can mean±1%.

This invention is further illustrated by the following examples which should not be construed as limiting. The contents of all references cited throughout this application, as well as the figures and tables are incorporated herein by reference.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Methods to Identify a Cancer Responsive to a p73/p63 Targeting Agent

In some embodiments, the methods disclosed herein encompass determining the expression or activity levels of a particular p73 isoforms and isoforms of p63 cancer cells, for example cancer cells from a subject with or at risk for developing a cancer.

One aspect of the present invention relates to a method to determine the likelihood of a p73/p63 targeting treatment being effective in a subject affected with, or at risk of developing cancer. In one embodiment the cancer is breast cancer of the triple-negative subtype. In one embodiment, the method comprises detecting the expression and/or activity of both the p63 gene and the p73 gene in the subject. In one embodiment, the method to determine the likelihood of a p73/p63 targeting treatment being effective in a subject affected with, or at risk of developing cancer comprises determining the expression or activity of a p63 isoform, for example a DNp63 isoform, and the expression or activity of a p73 such as TAp73 or DNp73 isoform. In particular, the presence of both a DNp63 isoform, and a p73 isoform such as TAp73 or identifies that a p73/p63 targeting treatment is likely to be effective in the subject.

In similar embodiments, the methods as disclosed herein enable identification of cancers that are responsive to a p73/p63 treatment, where a cancer is identified as cancer comprising cancer cells expressing or having the activity of both a DNp63 isoform and a p73 isoform, such as TAp73 or DNp73 isoforms.

In one embodiment, a p73/p63 targeting treatment is likely to be effective in the subject if a biological subject obtained from the subject comprises the expression level or activity level of DNp63 at a higher molar ratio than the expression or activity of p73 isoform such as TAp73 or DNp73. For example, if the level of a DNp63 isoform is at least 1.2 fold, or at least 1.5 fold or greater, for example greater than 2.0-fold, than the level of a p73 isoform, such as TAp73 or DNp73.

Methods to Identify a Cancer Unresponsive to a p73/p63 Targeting Agent

In another embodiment, the methods as disclosed herein relate to a method to identify if a cancer is unresponsive to a p73/p63 targeting treatment such as cisplatin, where a cancer unresponsive is identified as a cancer cell that does not express or have the activity of DNp63 isoforms. In such an embodiment, the method comprises measuring the expression and/or activity of at least one isoform of DNp63 in at least one cancer cell, wherein the absence of expression and/or activity of a DNp63 isoforms identifies the cancer as being more likely to be unresponsive to cisplatin or a derivative thereof, as compared to a cancer wherein the expression or the activity of DNp63 is detected.

In another embodiment, a cancer cell is identified as being unresponsive to a p73/p63 targeting treatment if the level of expression of DNp63 in the cancer cell below a level DNp63 of a reference level, for example where a reference level is, but not limited to a level of DNp63 in a cancer cell responsive to a p73/p63 targeting treatment such as cisplatin.

The term “biological sample” is intended to include tissues, cells and biological fluids isolated from a subject, as well as tissues, cells and fluids present within a subject. In some embodiments, the methods as disclosed herein provide for the detection of mRNA, protein, or genomic DNA of p63 isoforms such as DNp63 and/or p73 isoforms, such as TAp73 in a biological sample in vitro and/or in vivo and/or ex vivo. As disclosed herein, techniques for detecting gene expression of p63 isoforms such as DNp63, and/or p73 isoforms such as TAp73 include for example but are not limited to; Northern blot hybridizations, in situ hybridizations, QRT-PCR, PCR etc. As disclosed herein, techniques for detecting protein expression of p63 isoforms such as DNp63, and/or p73 isoforms such as TAp73 or DNp73, include for example but are not limited to; enzyme linked immunosorbent assays (ELISAs), Western blots, immunoprecipitations, immunofluorescence etc. Techniques for detecting genomic DNA sequences of p63 isoforms such as DNp63, and/or p73 isoforms such as TAp73, include for example but is not limited to, Southern hybridizations, PCR etc.

In some embodiments, the method also encompasses techniques for detecting protein expression of p63 isoforms such as DNp63, and/or p73 isoforms such as TAp73 or DNp73 isoforms in cancer cell in vivo. As a non-limiting example, the methods as disclosed here encompass introducing into a subject a labeled antibody or protein-binding molecule specific for isoforms of p63 such as DNp63 isoforms, and/or p73 isoforms such as TAp73 or DNp73. In some embodiments, the antibodies can be labeled with markers whose presence and location in a subject can be detected by standard imaging techniques, for example such markers, include but are not limited to radioactive, fluorescence and bioluminescence markers etc.

In one embodiment, the biological sample contains cancer cells from a subject.

In some embodiments, the methods further involve comparing the level of p63 isoforms such as DNp63 isoforms and p73 isoforms such as TAp73 or DNp73 against a reference level of DNp63 isoforms and p73 isoforms such as TAp73 or DNp73. In some embodiments, the reference level of DNp63 isoforms and p73 isoforms such as TAp73 or DNp73 is obtained from a control biological sample or a reference biological sample. In some embodiments, a reference or control biological sample comprise cells can be obtained from the same subject or a different subject, for example a biological sample of a physiologically matched tissue can comprise non-cancerous cells. Alternatively, a reference biological sample can be obtained from a reference or control subject. In some embodiments, a reference biological sample is contacted with an agent capable of interactions with the protein, mRNA, or genomic DNA of isoforms of p63 such as DNp63 isoforms and/or isoforms of p73 such as TAp73 or DNp73 isoforms, such that the level of such p63 isoforms and/or p73 isoforms protein, mRNA or genomic DNA detected in the reference biological sample can be compared with the levels of protein, mRNA or genomic DNA of p63 isoforms such as DNp63 isoforms and/or isoforms of p73 such as TAp73 or DNp73 isoforms in the test biological sample. In some embodiments, where a biological sample from a subject has a greater level of protein or gene expression of p63 isoforms such as DNp63 isoforms as compared to a reference biological sample, for example, if the level of DNp63 isoforms in the subjects biological sample is greater, for example at least 1.2-fold greater or at least 1.5 fold greater or at least two-fold more than the level of DNp63 isoforms in the reference biological sample, the cancer is identified to be likely to be responsive to p73/p63 targeting treatment.

In some embodiments, the methods as disclosed herein provide a diagnostic test for the activity of the proteins of p63 isoforms such as DNp63 isoforms and/or p73 isoforms such as TAp73 isoforms or DNp73. In some embodiments, p63 isoform and p73 isoform activity is transcriptional activity. In one embodiment, a diagnostic test useful in the methods as disclosed herein detects p73 isoform-mediated transcription of at least one p73 substrate and/or effector gene, such as NOXA or PUMA. Accordingly, in some embodiments, the levels of p73 isoform activity, such as TAp73 or DNp73 isoform activity can be assessed, and activity of a p73 isoform in response to a p73/p63 targeting treatment would indicate a p73/p63 targeting treatment would be an effective treatment in the subject. In some embodiments, detection of high levels of p73 isoforms such as TAp73 or DNp73 isoform or DNp73 isoform protein activity in cancer cells from a subject can be used as a diagnostic to identify cancers which are likely to be responsive to a p73/p63 targeting treatment. In related embodiments, the comparison of p73 isoform protein activity level, such as TAp73 or DNp73 can be determined between treated and untreated biopsy samples, cell lines, transgenic animals, or extracts from any of these, to determine the effect of a given treatment on p73 isoform activity as compared to an untreated control.

### Methods to Detect p73 and p63 Isoforms

In some embodiments, measurement of level of p63 isoforms such as DNp63 isoforms and level of p73 isoforms, such as TAp73 or DNp73 isoforms in a biological sample can be performed by any method commonly known by persons of ordinary skill in the art. For example, the methods as disclosed herein encompass methods to measure the level of DNp63 isoform and TAp73 isoform gene transcripts, such as levels of mRNA, in a biological sample. In alternative embodiments, methods to measure the level of DNp63 isoform and p73 isoform protein expression (i.e. gene product) in a biological sample are useful in the methods of the present invention. In another embodiment, methods to measure the level of DNp63 isoform and p73 isoform protein activity, for example phosphorylation of p73, in a biological sample are useful in the methods of the present invention.

In embodiments where the level of DNp63 isoform and p73 isoform gene transcripts, such as levels of mRNA, are measured in a biological sample, such measurements are commonly performed using DNA or RNA collected from biological samples, e.g., tissue biopsies, urine, stool, sputum, blood, cells, tissue scrapings, breast aspirates or other cellular materials, and can be performed by a variety of methods commonly known in the art, including, but not limited to, PCR, RT-PCR, quantitative RT-PCR (QRT-PCR), isoform-specific QRT-PCR, hybridization with isoform-specific probes.

In embodiments where the level of DNp63 isoform and TAp73 isoform protein expression are measured in a biological sample, such methods are commonly known in the art, and include, for example but not limited to; isoform-specific chemical or enzymatic cleavage of isoform proteins, immunobloting, immunohistochemical analysis, ELISA, and mass spectrometry.

### Detection of Gene Expression of p73 and p63 Isoforms

In some embodiments, determining the activity of p63 isoforms and p73 isoforms can be done by in vitro assays commonly known by a persons of ordinary skill in the art, for example Northern blot, RNA protection assay, microarray assay etc. In some embodiments, QRT-PCR can be conducted as ordinary QRT-PCR or as multiplex QRT-PCR assay where the assay enables the simultaneous detection of p63 isoforms and p73 isoforms, for example DNp63 isoforms and TAp73 isoforms, either together or separately from the same reaction sample. The methods as disclosed herein also encompass other diagnostic tests that involve a variety of different methods commonly known by person ordinary skill in the art.

Methods to detect gene expression for use in diagnostic tests are well known in the art and disclosed in International Patent Application WO200004194, which is incorporated herein in its entirety by reference. In one embodiment, the methods as disclosed herein provide a method to analyze gene expression for use in a diagnostic test comprises amplifying a segment of DNA or RNA (generally after converting the RNA to cDNA) spanning one or more known isoforms of p63 and p73 gene sequences. This amplified segment is then subjected to a detection method, such as signal detection, for example fluorescence, enzymatic etc. and/or polyacrylamide gel electrophoresis. Comparison of the PCR products by quantitative mean of the test biological sample to a control sample indicates different levels in the test biological sample. The change can be the same, increased or decreased level of expression of isoforms of p63 and isoforms of p73, for example DNp63 isoforms and TAp73 isoforms in the test biological sample.

In alternative embodiments, methods to determine expression of p63 isoforms and p73 isoforms can be established by methods involving QRT-PCR. In one embodiment, a comparison of the PCR products by quantitative means can be done by QRT-PCR, involving a reaction comprising the use of two primers for p63 isoforms and two primers for isoforms of p73. In another embodiment, a QRT-PCR involves the use of primers for DNp63 isoforms and/or primers for TAp73 isoforms.

As an alternative embodiment, a QRT-PCR reaction comprises of use of two primers for p63 isoforms and two primers for p73 isoforms, and a probe for hybridization to a p63 isoform and a probe for hybridization to a p73 isoform. In another embodiment, the primers and probes are specific for DNp63 isoforms and TAp73 isoforms.

In some embodiments, a probe useful in the methods as disclosed herein can comprise a nucleotide sequence of about 500 nucleotide bases, preferably about 100 nucleotides bases, and most preferably about 50 or about 25 bases or fewer in length. The probe may be composed of DNA, RNA, or peptide nucleic acid (PNA). Furthermore, the probe may contain a detectable label, such as, for example, a florescent or enzymatic label.

In some embodiments, where multiplex QRT-PCR assays are used, the expression level of isoforms of p63 and isoforms p73, preferably DNp63 isoforms and TAp73 or DNp73 isoforms, can be detected together with the expression level of one or more other proteins in the same reaction sample, for example but not limited to, endogenously expressed genes (for purposes of reference controls), other oncogenes, downstream effectors of p63 isoforms and/or p73 isoforms, such as, for example pro-apoptotic genes such as PUMA and NOXA. In another embodiment, isoform specific probes can be conducted in two formats: (1) allele specific oligonucleotides bound to a solid phase (glass, silicon, nylon membranes) and the labeled sample in solution, as in many DNA chip applications, or (2) bound sample (often cloned DNA or PCR amplified DNA) and labeled oligonucleotides in solution (either allele specific or short so as to allow sequencing by hybridization). In some embodiments, diagnostic tests may involve a panel of different probes, often but not limited to on a solid support, which enables the simultaneous determination of isoforms of p63 and/or isoforms of p73, with more than one other protein.

In alternative embodiments, amplification methods to detect levels of isoforms of p63 and p73 are encompassed for use in the methods as disclosed herein, and include for example but are not limited to: PCR, ligation chain reaction (LCR) (see, e.g., Landegran, et al., 1988. Science 24 1: 1 077-1 080; and Nakazawa, et al., 1994. Proc. Natl. Acad. Sci, USA 91:360-364), self sustained sequence replication (see, Guatelli, et al., 1990. Proc. Natl. Acad. Sci. USA 87: 1874-1 878), transcriptional amplification system (see, Kwoh, et al., 1989. Proc. Natl. Acad. Sci. USA 86: 1 173-1 177); Qb Replicase (see, Lizardi, et al, 1988. BioTechnology 6:1 197), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, the invention provides a method of screening for expression of p63 isoforms and p73 isoforms in a test biological sample by QRT-PCR.

The amplification products from the QRT-PCR can be analyzed by using any method known to the skilled artisan. In some embodiments, QRT-PCR methods comprise of techniques used to separate the amplification products according to size, including automated and manual gel electrophoresis, and signal intensity, such as an increase or decrease in a fluorescent or non-fluorescent signal signifies the presence or absence of an amplification product. The amplification products of p63 isoforms and p73 isoforms can be distinguished by different sizes and/or different signals, for example different florescent or non-fluorescent signals to signify presence or absence of the amplification products. The signals may be produced by a detectable label on the amplification product, consumable reagents or byproducts, such as, for example, a florescent or enzymatic label.

In some embodiments, primers and probes useful in the methods as disclosed herein can be designed using amino acid sequences of the protein and/or nucleic acid sequences of p63 isoforms such as DNp63 isoforms and p73 isoforms such as TAp73 or DNp73 isoforms by methods commonly known by persons of ordinary skill in the art. As a guide only, probes and primers can be designed based on complementary sequences to, e.g. SEQ ID NO:5 and SEQ ID NO:7 for isoforms of p63, and SEQ ID NO:1 and SEQ ID NO:3 for isoforms of p73. In some embodiments, primers are designed in the homologous regions of the gene wherein at least two regions of homology are separated by a divergent region of variable sequence, the sequence being variable either in length or nucleic acid sequence.

Probes useful in the methods as disclosed herein can be designed on the nucleotide sequence set forth in SEQ ID NO:1 is the nucleic acid sequence of the human TAp73 protein, deposited under GenBank database accession number NM_{—}005427. The amino acid sequence set forth in SEQ ID NO:2 is the amino acid sequence of the human TAp73 protein, deposited under GenBank database accession number NP_{—}005418. The nucleotide sequence set forth in SEQ ID NO:3 is the nucleic acid sequence of the human DNp73a protein, deposited under GenBank database accession number AB055065. The amino acid sequence set forth in SEQ ID NO:4 is the amino acid sequence of the human DNp73a protein, deposited under GenBank database accession number BAB87244.

As used herein, the nucleotide sequence set forth in SEQ ID NO:5 is the nucleic acid sequence of the human TAp63a protein, deposited under GenBank database accession number AF075430. The amino acid sequence set forth in SEQ ID NO:6 is the amino acid sequence of the human TAp63a protein, deposited under GenBank database accession number AAC62635. The nucleotide sequence set forth in SEQ ID NO:7 is the nucleic acid sequence of the human DNp63a (or protein DNp63α), corresponding to GenBank database accession number AF075431. The amino acid sequence set forth in SEQ ID NO:8 is the amino acid sequence of the human DNp63a protein, corresponding to GenBank database accession number AAC62636.

In some embodiments, a probe can be the nucleotide set forth in SEQ ID NO: 9 which is a nucleotide sequence for the forward primer for isoform specific QRT-PCR for TAp73, as disclosed herein. 5′-GCACCACGTTTGAGCACCTCT-3′ (SEQ ID NO: 9). In some embodiments, a probe can be the nucleotide set forth in SEQ ID NO: 10 5′-GCAGATTGAACTGGGCCATGA-3′ is the nucleotide sequence for the reverse primer for isoform specific QRT-PCR for TAp73 isoforms, as disclosed herein. In some embodiments, the probe can be the nucleotide set forth in SEQ ID NO: 11 is 5′-GGAAAACAATGCCCAGACTC-3′ a nucleotide sequence for the forward primer for isoform specific QRT-PCR for DNp63 isoforms, as disclosed herein. In some embodiments, a probe can be the nucleotide set forth in SEQ ID NO: 12 is 5′-GTGGAATACGTCCAGGTGGC-3′ a nucleotide sequence for the reverse primer for isoform specific QRT-PCR for DNp63 isoforms, as disclosed herein.

In further embodiments, the nucleotide set forth in SEQ ID NO: 13 is nucleotide sequence 5′-TGCTGATGGACTGCCAAAAA-3′ and SEQ ID NO:14 is nucleotide sequence 5′-TGGCTGCTCACTACTATCCAGAAC-3′ are useful in the methods as disclosed herein as the forward and reverse primers for QRT-PCR of p73 isoforms, respectively. Alternatively, the nucleotide set forth in SEQ ID NO: 15 is nucleotide sequence 5′-CCCTTACTGGCTTACCTCCTCAT-3′ and SEQ ID NO: 16 is nucleotide sequence 5′-CCCTTACCCTGGCTACTCATACA-3′ are useful in the methods as disclosed herein as forward and reverse primers for QRT-PCR of p63 isoforms, respectively.

In additional embodiments, the nucleotide set forth in SEQ ID NO: 17 is nucleotide sequence 5′-ATTTTGCGACATCTTT-3′ are useful in the methods as disclosed herein as hybridization probes for QRT-PCR for p73 isoforms. In alternative embodiments, the nucleotide set forth in SEQ ID NO: 18 is nucleotide sequence 5′-CAGCCTACTCTCCTTG-3′ are useful in the methods as disclosed herein as hybridization probe for QRT-PCR for p63 isoforms.

In some embodiments, where probes and primers are based on amino acid sequences, the can be designed based on a region identical to or highly homologous to, preferably at least 80%-85%, more preferably at least 90-99% homologous amino acid sequence of at least about 6, preferably at least 8-10 consecutive amino acids. In some embodiments, the amino acid sequence is 100% identical. In some embodiments, forward and reverse primers are designed based upon the maintenance of codon degeneracy and the representation of the various amino acids at a given position among the known gene family members. The degree of homology as referred to herein is based upon analysis of an amino acid sequence using standard sequence comparison software, such as protein-BLAST using the default settings (www.pcbi.n1rn.nih.gov/BLAST/).

In some embodiments, primers and probes useful in the methods as disclosed herein can be designed using a number of available computer programs, including, but not limited to Oligo Analyzer3.0; OligoCalculator; NetPrimer; Methprimer; Primer3; WebPrimer; PrimerFinder; Primer9; Oligo2002; Pride or GenomePride; Oligos; and Codehop. Detailed information about these programs can be obtained, for example, from www.molbiol.net.

In some embodiments, primers may be labeled using labels known to one skilled in the art. Such labels include, but are not limited to radioactive, fluorescent, dye, and enzymatic labels.

In some embodiments, analysis of amplification products can be performed using any method commonly known by persons of ordinary skill in the art, including for example, methods capable of separating the amplification products according to their signal intensity, size, including automated and manual gel electrophoresis, mass spectrometry, fluoresce spectrometers, and the like. Alternatively, amplification products can be separated using sequence differences, using SSCP, DGGE, TGGE, chemical cleavage or restriction fragment polymorphisms as well as hybridization to, for example, a nucleic acid arrays.

Where the level of p63 isoforms such as DNp63 isoforms and p73 isoforms such as TAp73 isoforms are detected at the level of gene transcription (i.e. mRNA level), methods of RNA isolation, RNA reverse transcription (RT) to cDNA (copy DNA) and cDNA or nucleic acid amplification and analysis that are routine for one skilled in the art can be used, and examples of protocols can be found, for example, in the Molecular Cloning: A Laboratory Manual (3-Volume Set) Ed. Joseph Sambrook, David W. Russel, and Joe Sambrook, Cold Spring Harbor Laboratory; 3rd edition (Jan. 15, 2001), ISBN: 0879695773. Particularly useful protocol source for methods used in PCR amplification is PCR (Basics: From Background to Bench) by M. J. McPherson, S. G. Møller, R. Beynon, C. Howe, Springer Verlag; 1st edition (Oct. 15, 2000), ISBN: 0387916008.

Other methods for detecting expression of p63 isoforms and p73 isoforms are encompassed for use in the methods as disclosed herein, and include for example, but not limited to, analyzing RNA expression comprise methods such as Northern blot, RNA protection assay, microarray assay etc. Such methods are well known in the art. In an exemplary embodiment, methods to detect p63 and p73 isoform expression comprises amplifying a segment of RNA (generally after converting the RNA to cDNA) which spans the gene sequences encoding one or more isoforms of p63 or one or more isoforms of p73. The amplified segment is then analyzed by appropriate methods to determine the presence and amount of amplified segment, for example analysis by semi-quantitative and quantitative measures.

In one embodiment, the expression or activation of p63 isoforms or p73 isoforms involves determining the activation status of downstream targets of p73 isoforms, and/or downstream targets of p63 isoforms, for example, as discovered herein, the inventors compared the expression of the major downstream targets of one of the TAp73 isoforms such as NOXA and PUMA (see Examples 5 and 6). Accordingly, examination of the expression of NOXA and PUMA is useful in determining the activation status of p73 and DNp63 isoforms.

### Detection of Protein Expression of p73 and p63 Isoforms

In another embodiment, the level of p63 isoforms or p73 isoforms can be determined by determining protein levels using immunological techniques commonly known by persons of ordinary skill in the art, e.g., antibody techniques such as immunohistochemistry, immunocytochemistry, FACS scanning, immunoblotting, radioimmunoassays, western blotting, immunoprecipitation, enzyme-linked immunosorbant assays (ELISA), and derivative techniques that make use of antibodies directed against proteins of p63 isoforms such as DNp63 and proteins of p73 isoforms, such as TAp73 or DNp73. In some embodiments, detection of proteins of activated downstream targets of isoforms of p73 such as, for example, detection of NOXA and PUMA is useful.

In some embodiments, detection of protein activation using phospho-specific antibodies, where active isoforms of p63 or isoforms of p73 can be detected and are useful as diagnostic indicators for the presence of p63 and/or p73 isoforms. In some embodiments, methods to detect activated isoforms of DNp63 and isoforms of p73 in a test biological sample can be performed by immunohistochemical or immunocytochemical methods.

In another embodiment, the presence of activated (phosphorylated) p73 isoforms may indicate p73/p63 targeting treatment is likely to be effective.

Any method to detect protein expression known by persons of ordinary skill in the art are useful in the methods as disclosed herein to detect the level of p63 isoforms such as DNp63 and p73 isoforms such as TAp73 or DNp73. For example, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of a specific antibody, wherein antibodies are used to specifically target molecules inside or on the surface of cells. The antibody typically contains a marker that will undergo a biochemical reaction, and thereby experience a change color, upon encountering the targeted molecules. In some instances, signal amplification may be integrated into the particular protocol, wherein a secondary antibody, that includes the marker stain, follows the application of a primary specific antibody.

Immunohistochemical assays are known to those of skill in the art (e.g., see Jalkanen, et al., J. Cell. Biol. 101:976-985 (1985); Jalkanen, et al., J. Cell. Biol. 105:3087-3096 (1987).

In some embodiments, antibodies, polyclonal, monoclonal and chimeric antibodies useful in the methods as disclosure herein can be purchased from a variety of commercial suppliers, or may be manufactured using well-known methods, e.g., as described in Harlow et al., Antibodies: A Laboratory Manual, 2nd Ed; Cold. Spring Harbor Laboratory Press, Cold Spring Harbor, N.Y. (1988). In general, examples of antibodies useful in the present invention include anti-p63, anti-p73, anti-NOXA, anti-PUMA, and anti-phospho-tyrosine antibodies. Such antibodies can be purchased, for example, from Sigma-Aldrich, CalBiochem, Abcam, Santa-Cruz Biotechnology, novus Bio, U.S. biologicals, Millipore, LifeSpan, Abnova, CellSignalling etc.

Typically, for immunohistochemistry, tissue obtained from a subject and fixed by a suitable fixing agent such as alcohol, acetone, and paraformaldehyde, is sectioned and reacted with an antibody. Conventional methods for immunohistochemistry are described in Harlow and Lane (Eds) (1988) In “Antibodies A Laboratory Manual”, Cold Spring Harbor Press, Cold Spring Harbor, N.Y.; Ausbel et al (Eds) (1987), in Current Protocols In Molecular Biology, John Wiley and Sons (New York, N.Y.). Biological samples appropriate for such detection assays include, but are not limited to, cells, tissue biopsy, whole blood, plasma, serum, sputum, cerebrospinal fluid, breast aspirates, pleural fluid, urine and the like.

In some embodiments, direct labeling techniques can be used, where a labeled antibody is utilized. For indirect labeling techniques, the sample is further reacted with a labeled substance.

In some embodiments, immunocytochemistry may be utilized where, in general, tissue or cells are obtained from a subject are fixed by a suitable fixing agent such as alcohol, acetone, and paraformaldehyde, to which is reacted an antibody. Methods of immunocytological staining of human samples is known to those of skill in the art and described, for example, in Brauer et al., 2001 (FASEB J, 15, 2689-2701), Smith Swintosky et al., 1997.

Immunological methods are particularly useful in the methods as disclosed herein, because they require only small quantities of biological material, and are easily performed and at multiple different locations. In some embodiments, such an immunological method useful in the methods as disclosed herein uses a “lab-on-a-chip” device, involving a single device to run a single or multiple biological samples and requires minimal reagents and apparatus and is easily performed, making the “lab-on-a-chip” devices which detect protein levels of p63 isoforms such as DNp63 and p73 isoforms such as TAp73 or DNp73 ideal for rapid, on-site diagnostic tests to identify a subject responsive to p73/p63 targeting treatments. In some embodiments, the immunological methods can be done at the cellular level and thereby necessitate a minimum of one cell, Preferably, several cells are obtained from a subject affected with or at risk for developing cancer and assayed according to the methods of the present invention.

In some embodiments, antibodies can be utilized to detect the presence of p63 isoform proteins such as DNp63 isoforms and/or p73 isoform proteins such as TAp73 or DNp73 isoforms, individually, or when they exist in p63:p73 complexes. In some embodiments, antibodies can bind to proteins individually or in a complex, and in some embodiments the antibody or antibodies are labeled with a detectable label.

In some embodiments, antibodies or protein-binding agents useful in the methods as disclosed herein bind or have affinity for DNp63 isoforms and/or TAp73 or DNp73 isoforms. If an antibody or protein-binding molecule that binds to p63 isoforms and/or p73 isoforms when they are present in a p63:p73 complex, a preferred antibody or protein-binding molecule is specific for DNp63 isoforms in complexes with any isoform of p73.

Antibodies useful in the methods as disclosed herein to detect the level of p63 isoforms such as DNp63 and p73, such as TAp73 or DNp73 can be polyclonal, monoclonal, chimeric antibodies, humanized antibodies, tribodies, midibodies, recombinant antibodies and any antibody, or fragment thereof, commonly known by persons of ordinary skill in the art. In some embodiments, an intact antibody, or a fragment thereof (e.g., Fab or F(ab)₂) can be used. Antibodies reactive to, or bind specifically to p63 isoforms such as DNp63 and p73, such as TAp73 or DNp73 can be readily raised in animals such as rabbits or mice by immunization with the antigen. Immunized mice are particularly useful for providing sources of B cells for the manufacture of hybridomas, which in turn are cultured to produce large quantities of monoclonal antibodies.

In one embodiment of this invention, the inhibitor to the gene products identified herein can be an antibody molecule or the epitope-binding moiety of an antibody molecule and the like. Antibodies provide high binding avidity and unique specificity to a wide range of target antigens and haptens. Monoclonal antibodies useful in the practice of the present invention include whole antibody and fragments thereof and are generated in accordance with conventional techniques, such as hybridoma synthesis, recombinant DNA techniques and protein synthesis.

Useful monoclonal antibodies and fragments can be derived from any species (including humans) or can be formed as chimeric proteins which employ sequences from more than one species. Human monoclonal antibodies or “humanized” murine antibody are also used in accordance with the present invention. For example, murine monoclonal antibody can be “humanized” by genetically recombining the nucleotide sequence encoding the murine Fv region (i.e., containing the antigen binding sites) or the complementarily determining regions thereof with the nucleotide sequence encoding a human constant domain region and an Fc region. Humanized targeting moieties are recognized to decrease the immunoreactivity of the antibody or polypeptide in the host recipient, permitting an increase in the half-life and a reduction the possibly of adverse immune reactions in a manner similar to that disclosed in European Patent Application No. 0,411,893 A2. The murine monoclonal antibodies should preferably be employed in humanized form. Antigen binding activity is determined by the sequences and conformation of the amino acids of the six complementarily determining regions (CDRs) that are located (three each) on the light and heavy chains of the variable portion (Fv) of the antibody. The 25-kDa single-chain Fv (scFv) molecule, composed of a variable region (VL) of the light chain and a variable region (VH) of the heavy chain joined via a short peptide spacer sequence, is the smallest antibody fragment developed to date. Techniques have been developed to display scFv molecules on the surface of filamentous phage that contain the gene for the scFv. scFv molecules with a broad range of antigenic-specificities can be present in a single large pool of scFv-phage library. Some examples of high affinity monoclonal antibodies and chimeric derivatives thereof, useful in the methods of the present invention, are described in the European Patent Application EP 186,833; PCT Patent Application WO 92/16553; and U.S. Pat. No. 6,090,923, which are incorporated herein in their entirety by reference.

Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined.

One limitation of scFv molecules is their monovalent interaction with target antigen. One of the easiest methods of improving the binding of a scFv to its target antigen is to increase its functional affinity through the creation of a multimer. Association of identical scFv molecules to form diabodies, triabodies and tetrabodies can comprise a number of identical Fv modules. These reagents are therefore multivalent, but monospecific. The association of two different scFv molecules, each comprising a VH and VL domain derived from different parent Ig will form a fully functional bispecific diabody. A unique application of bispecific scFvs is to bind two sites simultaneously on the same target molecule via two (adjacent) surface epitopes. These reagents gain a significant avidity advantage over a single scFv or Fab fragments. A number of multivalent scFv-based structures has been engineered, including for example, miniantibodies, dimeric miniantibodies, minibodies, (scFv)₂, diabodies and triabodies. These molecules span a range of valence (two to four binding sites), size (50 to 120 kDa), flexibility and ease of production. Single chain Fv antibody fragments (scFvs) are predominantly monomeric when the VH and VL domains are joined by, polypeptide linkers of at least 12 residues. The monomer scFv is thermodynamically stable with linkers of 12 and 25 amino acids length under all conditions. The noncovalent diabody and triabody molecules are easy to engineer and are produced by shortening the peptide linker that connects the variable heavy and variable light chains of a single scFv molecule. The scFv dimers are joined by amphipathic helices that offer a high degree of flexibility and the miniantibody structure can be modified to create a dimeric bispecific (DiBi) miniantibody that contains two miniantibodies (four scFv molecules) connected via a double helix. Gene-fused or disulfide bonded scFv dimers provide an intermediate degree of flexibility and are generated by straightforward cloning techniques adding a C-terminal Gly4Cys sequence. scFv-CH3 minibodies are comprised of two scFv molecules joined to an IgG CH3 domain either directly (LD minibody) or via a very flexible hinge region (Flex minibody). With a molecular weight of approximately 80 kDa, these divalent constructs are capable of significant binding to antigens. The Flex minibody exhibits impressive tumor localization in mice. Bi- and tri-specific multimers can be formed by association of different scFv molecules. Increase in functional affinity can be reached when Fab or single chain Fv antibody fragments (scFv) fragments are complexed into dimers, trimers or larger aggregates. The most important advantage of multivalent scFvs over monovalent scFv and Fab fragments is the gain in functional binding affinity (avidity) to target antigens. High avidity requires that scFv multimers are capable of binding simultaneously to separate target antigens. The gain in functional affinity for scFv diabodies compared to scFv monomers is significant and is seen primarily in reduced off-rates, which result from multiple binding to two or more target antigens and to rebinding when one Fv dissociates. When such scFv molecules associate into multimers, they can be designed with either high avidity to a single target antigen or with multiple specificities to different target antigens. Multiple binding to antigens is dependent on correct alignment and orientation in the Fv modules. For full avidity in multivalent scFvs target, the antigen binding sites must point towards the same direction. If multiple binding is not sterically possible then apparent gains in functional affinity are likely to be due the effect of increased rebinding, which is dependent on diffusion rates and antigen concentration. Antibodies conjugated with moieties that improve their properties are also contemplated for the instant invention. For example, antibody conjugates with PEG that increases their half-life in vivo can be used for the present invention. Immune libraries are prepared by subjecting the genes encoding variable antibody fragments from the B lymphocytes of naive or immunized animals or patients to PCR amplification. Combinations of oligonucleotides which are specific for immunoglobulin genes or for the immunoglobulin gene families are used. Immunoglobulin germ line genes can be used to prepare semisynthetic antibody repertoires, with the complementarity-determining region of the variable fragments being amplified by PCR using degenerate primers. These single-pot libraries have the advantage that antibody fragments against a large number of antigens can be isolated from one single library. The phage-display technique can be used to increase the affinity of antibody fragments, with new libraries being prepared from already existing antibody fragments by random, codon-based or site-directed mutagenesis, by shuffling the chains of individual domains with those of fragments from naive repertoires or by using bacterial mutator strains.

Alternatively, a SCID-hu mouse, for example the model developed by Genpharm, can be used to produce antibodies, or fragments thereof. In one embodiment, a new type of high avidity binding molecule, termed peptabody, created by harnessing the effect of multivalent interaction is contemplated. A short peptide ligand was fused via a semirigid hinge region with the coiled-coil assembly domain of the cartilage oligomeric matrix protein, resulting in a pentameric multivalent binding molecule. In some embodiments, proteins-binding agents can be targeted to tissue- or tumor-specific targets by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. Alternatively in some embodiments, two or more protein-binding molecules can be administered, for example in some embodiments a protein binding molecule can be an antibody that is conjugated to another a different antibody. Each antibody is reactive with a different target site epitope (associated with the same or a different target site antigen). The different antibodies with the agents attached accumulate additively at the desired target site. Antibody-based or non-antibody-based targeting moieties can be employed to deliver a ligand or the inhibitor to a target site. Preferably, a natural binding agent for an unregulated or disease associated antigen is used for this purpose.

In some embodiments, antibodies and protein-binding molecules are labeled. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin.

Methods to Treat a Subject Identified to be Responsive to a p73/p63 Treatment.

Exemplarily examples of p73/p63 targeting treatments are for example but not limited to, chemotherapeutic agents, such as platinum based chemotherapeutic agents. Examples include, but are not limited to cisplatin (cis-diaminedichloroplatinuim (II), cis-DDP, CDDP), cisplatin compounds, cisplatin metabolites, derivatives or analogues thereof having a skeleton similar to cisplatin. Analogues of cisplatin, for example include, but are not limited to, carboplaitin (cis-diamine[1,1-cyclobutnaedicarboxylate(2-)-O,O′-platinum(II)) and oxaliplatin (cis-L-diaminocyclohexane oxalotoplatinum (II). Cisplatin derivatives include, for example but not limited to, those set forth in U.S. Patent Application No: US2006/0142593, which is incorporated herein in its entirety by reference.

Chemotherapeutic Agents

According to the invention, antisense constructs of the invention and other antagonists may be employed to increase the sensitivity (decrease the non-responsiveness) of tumor cells to p73/p63 targeting treatments. In one embodiment, the p73/p63 targeting treatment is a chemotherapeutic agent, such as platinum based chemotherapeutic agents. Several platinum based agents have been used as chemotherapeutic agents. For the purposes of the invention, examples of drugs include, but are not limited to cisplatin (cis-diaminedichloroplatinuim (II), cis-DDP, CDDP), cisplatin compounds, cisplatin metabolites, derivatives or analogues thereof having a skeleton similar to cisplatin.

Analogues of cisplatin, for example include, but are not limited to, carboplaitin (cis-diamine[1,1-cyclobutnaedicarboxylate(2-)-O,O′-platinum(II)) and oxaliplatin (cis-L-diaminocyclohexane oxalotoplatinum (II). Cisplatin derivatives include, for example but not limited to, those set forth in U.S. Patent No: US2006/0142593 which is incorporated herein in its entirety by reference.

In another embodiment of the invention, antagonists to specific p63 isoforms, in particular DNp63 isoforms can be used as chemotherapy agents to activate p73 isoforms, enabling the p73 isoforms to activate the transcription of downstream pro-apoptotic effector molecules.

Those skilled in the art will appreciate that a p73/p63 targeting treatment to which the present invention refers are not limited to the above-mentioned specific agents but include any compound or entity that functions as a p73/p63 targeting treatment.

Antagonists or Inhibitor Agents to p63 Isoforms and DNp63 Isoforms

Additional embodiments provide methods and compositions for inhibiting the expression or activity of p63 isoforms using an antagonist thereof. In a particular embodiment, antagonists to p63 isoforms are antagonists to DNp63 isoforms. P63 isoform antagonists may be a nucleic acid-based inhibitor, nucleic acid construct, a peptide-based inhibitor or a small molecule inhibitor of p63 isoforms or DNp63 isoforms or the polynucleotide encoding the same. The nucleic-acid inhibitor may be a siRNA molecule or an antisense construct.

In some embodiments, agents which inhibit P63 isoforms, such as DNp63 isoforms can be, for example but not limited to, antibodies (polyclonal or monoclonal), neutralizing antibodies, antibody fragments, peptides, proteins, peptide-mimetics, aptamers, oligonucleotides, hormones, small molecules, nucleic acids, nucleic acid analogues, carbohydrates or variants thereof that function to inactivate the nucleic acid and/or protein of the gene products identified herein, and those as yet unidentified. Nucleic acids include, for example but not limited to, DNA, RNA, oligonucleotides, peptide nucleic acid (PNA), pseudo-complementary-PNA (pcPNA), locked nucleic acid (LNA), RNAi, microRNAi, siRNA, shRNA etc. The inhibitors can be selected from a group of a chemical, small molecule, chemical entity, nucleic acid sequences, nucleic acid analogues or protein or polypeptide or analogue or fragment thereof. In some embodiments, the nucleic acid is DNA or RNA, and nucleic acid analogues, for example can be PNA, pcPNA and LNA. A nucleic acid may be single or double stranded, and can be selected from a group comprising; nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide inhibitor or fragment thereof, can be, for example, but not limited to mutated proteins; therapeutic proteins and recombinant proteins. Proteins and peptides inhibitors can also include for example; mutated proteins, genetically modified proteins, peptides, synthetic peptides, recombinant proteins, chimeric proteins, antibodies, humanized proteins, humanized antibodies, chimeric antibodies, modified proteins and fragments thereof.

In some embodiments, the present invention relates to the inhibition of p63 isoforms, such as DNp63 isoforms. In some embodiments, inhibition is inhibition of nucleic acid transcripts encoding p63 isoforms, such as DNp63 isoforms, for example inhibition of messenger RNA (mRNA). In alternative embodiments, inhibition of p63 isoforms, such as DNp63 isoforms is inhibition of the expression and/or inhibition of activity of the gene product of p63 isoforms, such as DNp63 isoforms, for example the polypeptide or protein of p63 isoforms, such as DNp63 isoforms, or isoforms thereof. As used herein, the term “gene product” refers to RNA transcribed from a gene, or a polypeptide encoded by a gene or translated from RNA.

In some embodiments, inhibition of p63 isoforms, such as DNp63 isoforms is by an agent. One can use any agent, for example but are not limited to nucleic acids, nucleic acid analogues, peptides, phage, phagemids, polypeptides, peptidomimetics, ribosomes, aptamers, antibodies, small or large organic or inorganic molecules, or any combination thereof. In some embodiments, agents useful in methods of the present invention include agents that function as inhibitors of p63 isoforms, such as DNp63 isoforms expression, for example inhibitors of mRNA encoding p63 isoforms, such as DNp63 isoforms.

Agents useful in the methods as disclosed herein can also inhibit gene expression (i.e. suppress and/or repress the expression of the gene). Such agents are referred to in the art as “gene silencers” and are commonly known to those of ordinary skill in the art. Examples include, but are not limited to a nucleic acid sequence, for an RNA, DNA or nucleic acid analogue, and can be single or double stranded, and can be selected from a group comprising nucleic acid encoding a protein of interest, oligonucleotides, nucleic acids, nucleic acid analogues, for example but are not limited to peptide nucleic acid (PNA), pseudo-complementary PNA (pc-PNA), locked nucleic acids (LNA) and derivatives thereof etc. Nucleic acid agents also include, for example, but are not limited to nucleic acid sequences encoding proteins that act as transcriptional repressors, antisense molecules, ribozymes, small inhibitory nucleic acid sequences, for example but are not limited to RNAi, shRNAi, siRNA, micro RNAi (miRNA), antisense oligonucleotides, etc.

As used herein, agents useful in the method as inhibitors of p63 isoforms, such as DNp63 isoforms expression and/or inhibition of P63 isoforms, such as DNp63 isoforms protein function can be any type of entity, for example but are not limited to chemicals, nucleic acid sequences, nucleic acid analogues, proteins, peptides or fragments thereof. In some embodiments, the agent is any chemical, entity or moiety, including without limitation, synthetic and naturally-occurring non-proteinaceous entities. In certain embodiments the agent is a small molecule having a chemical moiety. For example, in some embodiments, the chemical moiety is a pyrimidione-based compound as disclosed herein.

In alternative embodiments, agents useful in the methods as disclosed herein are proteins and/or peptides or fragment thereof, which inhibit the gene expression of p63 isoforms, such as DNp63 isoforms or the function of the p63 isoforms, such as DNp63 isoforms protein. Such agents include, for example but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof.

Alternatively, agents useful in the methods as disclosed herein as inhibitors of p63 isoforms, such as DNp63 isoforms can be a chemicals, small molecule, large molecule or entity or moiety, including without limitation synthetic and naturally-occurring non-proteinaceous entities. In certain embodiments the agent is a small molecule having the chemical moieties as disclosed herein.

In particular embodiments the antagonist is a nucleic-acid based inhibitor of expression of polynucleotide encoding p63 isoforms or fragments thereof. Suitable molecules include small interfering RNA (siRNA) species, antisense constructs, such as antisense oligonucleotides, and catalytic antisense nucleic acid constructs. Suitable molecules can be manufactured by chemical synthesis, recombinant DNA procedures or, in the case of antisense RNAi by transcription in vitro or in vivo when linked to a promoter, by methods known to those skilled in the art.

One suitable technology for inhibiting gene expression, known as RNA interference (RNAi), (see, e.g. Chuang et al. (2000) PNAS USA 97: 4985) may be used for the purposes of the present invention, according to known methods in the art (for example Fire et al. (1998) Nature 391: 806-811; Hammond, et al. (2001) Nature Rev, Genet. 2: 110-1119; Hammond et al. (2000) Nature 404:293-296; Bernstein et al. (2001) Nature 409: 363-366; Elbashir et al (2001) Nature 411: 494-498; WO 99/49029 and WO 01/70949, the disclosures of which are incorporated herein by reference), to inhibit the expression of p63 isoforms. RNAi refers to a means of selective post-transcriptional gene silencing by destruction of specific mRNA by small interfering RNA molecules (siRNA). The siRNA is typically generated by cleavage of double stranded RNA, where one strand is identical to the message to be inactivated. Double-stranded RNA molecules may be synthesized in which one strand is identical to a specific region of the mRNA transcript of the p63 isoform of interest and introduced directly. Alternatively corresponding double stranded DNA (dsDNA) can be employed, which can be converted into dsRNA. Methods for the synthesis of suitable siRNA molecules for use in RNAi and for achieving post-transcriptional gene silencing are known to those of skill in the art. Those skilled in the art will also appreciate that a range of suitable siRNA constructs capable of inhibiting the expression of specific p63 isoforms can be identified and generated based on knowledge of the sequence of the gene in question using routine procedures known to those skilled in the art without undue experimentation.

The isolated inhibitory nucleic acid construct comprising a nucleic acid sequence specific to a least a portion of the polynucleotide encoding DNp63 isoforms, wherein the nucleic acid construct substantially inhibits the expression of isoforms of DNp63 in tumor cells. Alternatively, inhibitory nucleic acid constructs may comprise of a nucleic acid sequences specific to at least a portion of a polynucleotide encoding one or more genes which regulate the expression of isoforms of p63 or isoforms of DNp63. Genes that regulate the expression of isoforms of p63 or isoforms of DNp63 comprise, for example, but not limited to, transcription factors, co-activators, activators, enhancers and cofactors of p63 isoforms and/or DNp63 isoforms.

An example of a siRNA as used herein is the nucleotide set forth in SEQ ID NO: 19 is 5′-GGATTCCAGCATGGACGTCTT-3′ and is a nucleotide sequence of the RNAi molecule to TAp73 isoforms, and gene silences TAp73 expression.

The nucleotide set forth in SEQ ID NO: 20 is 5′-GAGTGGAATGACTTCAACTTT-3′ and SEQ ID NO:21 is 5′-GGGTGAGCGTGTTATTGATGCT-3′ are nucleotide sequence of the RNAi molecules to p63 isoforms. SEQ ID NO:20 is referred to as RNAi sequence “p63si-1” or “p63si” used interchangeably herein, targets p63 isoforms; TAp63, DNp63alpha, TAp63β and DNp63β isoforms of p63 (but p637 isoforms), and SEQ ID NO: 21 is referred to as RNAi sequence “p63si-2”, targets TAp63α and DNp63α isoforms. In some embodiments, both SEQ ID NO: 21 and/or SEQ ID NO:20 are useful in the methods as disclosed herein as a DNp63 antagonist or inhibitor.

The portion of the polynucleotide may include the coding region of the gene encoding p63 isoforms or DNp63 isoforms, for example SEQ ID NO: 5 and SEQ ID NO:7 respectively, and/or one or more regulatory regions of the gene known to one skilled in the art.

The nucleic acid construct may be a siRNA molecule. In one embodiment, antagonists to DNp63 isoforms include the nucleotide sequence of a siRNA molecule comprising SEQ ID NO: 20 and/or SEQ NO: 21 or any other nucleotide sequence designed from SEQ ID NO:7 that is specific to isoforms of DNp63.

The nucleic acid construct may have a nucleotide sequence having at least 85% identity to the nucleotide sequence set forth in any one of SEQ ID NO: 20 and/or SEQ ID NO:21 or a fragment thereof.

In other embodiments, the nucleotide sequence may have at least 85%, at least 90% identity, or at least 95% identity, to the nucleotide sequence set forth in any one of SEQ ID NO: 20 and/or SEQ ID NO:21 or a fragment thereof.

Those skilled in the art will appreciate that there need not necessarily be 100% nucleotide sequence match between the target sequence and the siRNA sequence. The capacity for mismatch there between is dependent largely on the location of the mismatch within the sequences.

In particular embodiments of the invention suitable inhibitory nucleic acid molecules may be administered to the tumor cells in a vector. The vector may be a plasmid vector, a viral vector, or any other suitable vehicle adapted for the insertion and foreign sequence and for the introduction into eukaryotic cells. The vector can be an expression vector capable of directing the transcription of the DNA sequence of inhibitory nucleic acid molecules into RNA. Viral expression vectors can be selected from a group comprising, for example, reteroviruses, lentiviruses, Epstein Barr virus-, bovine papilloma virus, adenovirus- and adeno-associated-based vectors or hybrid virus of any of the above. In one embodiment, the vector is episomal. The use of a suitable episomal vector provides a means of maintaining the inhibitory nucleic add molecule in the tumor cells in high copy number extra chromosomal DNA thereby eliminating potential effects of chromosomal integration.

A further means of substantially inhibiting the expression specific p63 isoforms may be achieved by introducing catalytic antisense nucleic acid constructs, such as ribozymes, which are capable of cleaving RNA transcripts and thereby preventing the production of wildtype protein. Ribozymes are targeted to and anneal with a particular sequence by virtue of two regions of sequence complementary to the target flanking the ribozyme catalytic site. After binding the ribozyme cleaves the target in a site specific manner. The design and testing of ribozymes which specifically recognize and cleave sequences of isoforms of p63 can be achieved by techniques well known to those in the art (for example Lleber and Strauss, (1995) Mol Cell Biol 15:540.551, the disclosure of which is incorporated herein by reference).

Alternative antagonists of specific p63 isoforms may include antibodies. Suitable antibodies include, but are not limited to polyclonal antibodies, monoclonal antibodies, chimeric antibodies, humanized antibodies, human antibodies, single chain antibodies and Fab fragments.

Antibodies may be prepared from discrete regions of fragments of the polypeptide of interest. An antigenic polypeptide contains at least about 5, and preferably at least about 10 amino acids.

Methods for the generation of suitable antibodies will be readily appreciated by those skilled in the art. For example, a suitable monoclonal antibody, typically containing Fab portions, may be prepared using the hybridoma technology described in Antibodies—A Laboratory Manual Harlow and Lane, Eds. Cold Spring Harbor Laboratory, N.Y. (1988), the disclosure of which is incorporated herein by reference.

Similarly, there are various procedures known in the art which may be used for the production of polyclonal antibodies to polypeptides of interest as disclosed herein. For the production of polyclonal antibodies, various host animals, including but not limited to rabbit mice, rats, sheep, goats, etc, can be immunized by injection with a polypeptide, or fragment or analogue thereof. Further, the polypeptide or fragment or analogue thereof can be conjugated to an immunogenic carrier, e.g., bovine serum albumin (BSA) or keyhole limpet hemocyanin (KLH). Also, various adjuvants may be used to increase the immunological response, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacillus Calmette-Guerin) and Corynebacterium parvum.

Screening for the desired antibody can also be accomplished by a variety of techniques known in the art. Assays for immunospecific binding of antibodies may include, but are not limited to, radioimmunoassays, ELISAs (enzyme-linked immunosorbent assay), sandwich immunoassays, immunoradiometric assays, gel diffusion precipitation reactions, immunodiffusion assays, in situ immunoassays, Western blots, precipitation reactions, agglutination assays, complement fixation assays, immunofluorescence assays, protein A assays, and Immunoelectrophoresis assays, and the like (see, for example, Ausubel et al., eds, 1994, Current Protocols in Molecular Biology, Vol. 1, John Wiley & Sons, Inc., New York). Antibody binding may be detected by virtue of a detectable label on the primary antibody. Alternatively, the primary antibody may be detected by virtue of its binding with a secondary antibody or reagent which is appropriately labeled. Numerous methods are known by persons of ordinary skill in the art to detecting binding in an immunoassay and are within the scope of the present invention.

Also included within the scope of the present invention are alternative forms to inhibit the expression of specific p63 isoforms, including, for example, small molecule or other non-nucleic acid or non-proteinaceous inhibitors. Such inhibitors may be identified by those skilled in the art by screening using routine techniques.

Selection of Subjects Amenable to Determining their Responsiveness to a p73/p63 Treatment.

Embodiments of the invention provide methods for the determination of the likelihood of a p73/p63 targeting treatment to be effective, predicted on the inventor's finding of the co-expression or presence of activity of both p73 isoforms and p63 isoforms, in particular DNp63 isoforms in cancer tissue. Subjects amenable to testing for levels or activity of p63, such as DNp63 isoforms and p73 isoforms such as TAp73 or DNp73 using the methods as disclosed herein include subjects at risk of a cancer, as well as subjects at risk of developing cancer.

In one embodiment, the cancer tissue is breast cancer of the triple-negative subtype. Embodiments of the invention also provide methods for altering the sensitivity (i.e. increasing the sensitivity) of a tumor cell to p73/p63 targeting treatments, in particular cisplatin, by administering antagonists to p63 isoforms, in particular DNp63 isoforms. Also encompassed in the invention are methods to treat cancers by administering antagonists to specific p63 isoforms, in particular DNp63 isoforms. In such an embodiment, inhibition of specific p63 isoforms enables dissociation of active p73 isoforms, and thus transcription activation of downstream pro-apoptotic effector molecules.

Accordingly, the methods of the invention relate to the analysis and treatment of a variety of tumor cell types, to p73/p63 targeted treatments. For example, the tumor cell types can be selected from a group comprising of gastrointestinal cancer, gastric cancer, squamous cell carcinomas (SCC), head and neck cancer, lung cancer, non-small cell lung cancer (NSCLC) and small-cell lung cancer (SCLC), lymphoma, sarcoma, primary and metastic melanoma, thymoma, non-Hodgkin's lymphoma, Hodgkin's lymphoma, cancer of the nervous system, brain cancer, bone-marrow cancer, bone cancer, kidney cancer, uterine cancer, cervival cancer, colon cancer, retina cancer, skin cancer, bladder cancer, colon cancer, esophageal cancer, testicular cancer, cervical cancer, liver cancer, renal cancer, pancreatic cancer, genital-urinary cancer, gastrointestinal, gum cancer, tongue cancer, kidney cancer, nasopharynx cancer, stomach cancer, endometrial cancer and bowel tumor cell cancer, adrenocarcinomas such as prostate cancer, ovarian cancer, breast cancer, and pancreatic cancer.

In some embodiments, subjects amenable to testing for protein or gene levels or activity of p63, such as DNp63 isoforms and p73 isoforms such as TAp73 or DNp73 using the methods as disclosed herein include subjects with breast cancer, in particular the triple negative subtype breast cancer, which is characterized by ER/PR-negative also lacking HER2 expression. In alternative embodiments, subjects amenable to testing using the methods as disclosed herein are subjects with squamous cell carcinomas (SCC) or prostate cancer.

In some embodiments, subjects amenable to testing for protein or gene levels or activity of p63, such as DNp63 isoforms and p73 isoforms such as TAp73 or DNp73 using the methods as disclosed herein include any subject currently being administered or about to be administered a p73/p63 treatment, such as such as cisplatin or cisplatin derivatives or mimetics thereof. In alternative embodiments, subjects amenable to the diagnostic tests as disclosed herein to measure the protein or gene levels or activity of p63, such as DNp63 isoforms and p73 isoforms such as TAp73 or DNp73, include any subject that has been administered a p73/p63 treatment, such as cisplatin or a cisplatin derivatives, in the past and was found that such treatment was not effective, or the subject is, or has had cancer remission. Testing of such subjects using the methods as disclosed herein is useful to determine if the failure of the prior administration of a p73/p63 targeting treatment was due to only the expression of one of either DNp63 or p73, and thus identifies a subject not likely to be responsive to such a p73/p63 treatment. Accordingly, a physician can direct such subjects to be administered an alternative treatment regime not involving a p73/p63 targeting treatment in future cancer treatments or prophylactic cancer treatments.

In some embodiments, subjects amenable to testing for levels or activity of p63, such as DNp63 isoforms and p73 isoforms using the methods as disclosed herein include are adult and pediatric oncology subjects which have cancers such as solid phase tumors/malignancies, locally advanced tumors, human soft tissue sarcomas, metastatic cancer, including lymphatic metastases, blood cell malignancies including multiple myeloma, acute and chronic leukemias, and lymphomas, head and neck cancers including mouth cancer, larynx cancer and thyroid cancer, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, pancreatic cancers, liver cancer, urologic cancers including bladder cancer and prostate cancer, malignancies of the female genital tract including ovarian carcinoma, uterine (including endometrial) cancers, and solid tumor in the ovarian follicle, kidney cancers including renal cell carcinoma, brain cancers including intrinsic brain tumors, neuroblastoma, askocytic brain tumors, gliomas, metastatic tumor cell invasion in the central nervous system, bone cancers including osteomas, skin cancers including malignant melanoma, tumor progression of human skin keratinocytes, squamous cell carcinoma, basal cell carcinoma, hemangiopericytoma and Kaposi's sarcoma.

In some embodiments, subjects amenable to testing for levels or activity of p63, such as DNp63 isoforms and p73 isoforms such as TAp73 or DNp73 using the methods as disclosed herein include subjects with cancers such as, but are not limited to, bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS associated leukemias and adult T-cell leukemia lymphoma; intraepithelial neoplasms including Bowen's disease and Paget's disease, liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilm's tumor.

In some embodiments, subjects amenable to testing for levels or activity of p63, such as DNp63 isoforms and p73 isoforms such as TAp73 or DNp73 using the methods as disclosed herein include subjects identified with or having increased risk of cancer, for example subjects identified to carry a genetic mutation or polymorphism associated with an increase risk of developing cancer. Such mutations and genetic susceptibility genes and loci are commonly known by persons skilled in the art, for example some of the more commonly known genes where a mutation is associated with increase in cancer include, but are not limited to; BRAC1, BRAC2, EGFR, EIF4A2, ERBB2, RB1, CDKN2A., P53, INK4a, APC, MLH1, MSH2, MSH6, WTI, NF1, NF2, and VHL (see http://www.cancer.org/docroot/ETO/content/ETO_{—}1_{—}4x_oncogenes_and_tumor_suppressor_genes.as P).

In some embodiments, subjects amenable to determination of levels or activity of p63, such as DNp63 isoforms and p73 isoforms have been identified to have cancer as determined by a number of cancer screens commonly known by persons of ordinary skill in the art, for example a number of biochemical and genetic markers or other biomarkers. Biomarkers are defined as cellular, biochemical, molecular or genetic alterations by which a normal, abnormal or simply biologic process can be recognized or monitored. Biomarkers are measurable in biological media, such as human tissues, cells or fluids. Biomarkers could be used to identify pathological processes before individuals become symptomatic or to identify individuals who are responsive to cancer.

Several classes of biomarkers in cancer cells and bodily fluids have been studied, mostly in laboratories examining specific observations but also in limited clinical settings. Several biomarkers have shown only limited utility: e.g., CD44, telomerase, transforming growth factor-α (TGF-α)3, transforming growth factor-β (TGF-β), epidermal growth factor receptor erbB-2 (erbB-2), epidermal growth factor receptor erbB-3 (erbB-3), mucin 1 (MUC1), mucin 2 (MUC2) and cytokeratin 20 (CK20). Other biomarkers are used in clinical practice and include, for example Prostate specific antigen (PSA) and cancer antibody or tumor marker 125 (CA125). Several protein markers can be used as cancer biomarkers, for example but not limited to, Fecal occult blood test (FOBT), which is a protein biomarker shown to decrease cause-specific mortality in cancer screens.

In one embodiment, subjects amenable to determination of levels or activity of p63, such as DNp63 isoforms and p73 isoforms using the methods as disclosed herein include subjects with a high level of p73 isoforms such as TAp73 or DNp73 isoforms in a biological sample from the subject as compared to a reference level of such p73 isoforms, and thus have increased chance of a p73/p63 targeting treatment being effective if the biological sample also comprises p63 isoforms such as DNp63. If for example, the level of p73 isoforms such as TAp73 or DNp73 isoforms in a biological sample from the subject is above a reference level, the subject has increased chance of a p73/p63 targeting treatment being effective if the biological sample also comprises p63 isoforms such as DNp63. In some embodiments, the biological sample obtained from the subject is from a biopsy tissue sample, and in some embodiments, the sample is from a tumor or cancer tissue sample. The level of p73 isoforms such as TAp73 or DNp73 isoforms can be determined by methods as disclosed herein and include, without limitation known, any method by the skilled artisan, for example by northern blot analysis or RT-PCR. In some embodiments, the reference level of p73 isoforms is the level of p73 isoforms in a normal tissue sample, where in the tissue sample is a biological tissue sample from a tissue matched, species matched and age matched biological sample. In some embodiments, the reference level of p73 isoforms is based on a biological sample is from a non-malignant matched tissue sample. In some embodiments, the reference level of p73 isoforms is based on a biological sample from a non-stem cell cancer tissue sample.

Administration of p73/p63 Targeting Treatment to the Subject

Embodiments of the present invention relate to the use of antagonists of the invention in methods and compositions for treating individuals having a disease, the individual either being in need of, or undergoing p73/p63 targeting treatment. Another important embodiment of the present invention contemplates the administration of antagonist of the invention, such as antagonists to DNp63 isoforms, resulting in activation of p73 isoforms to induce cell death signaling effector molecules, to individuals having cancer.

Accordingly, antagonists of the invention may be administered in combination therapy with other p73/p63 targeting agent, therapeutic interventions, or anti-cancer treatments. For such combination therapies, each component of the combination therapy may be administered at the same time, or sequentially, in any order, or at different times, so as to provide the desired therapeutic effect. One or more suitable antagonist may be combined with one or more suitable p73/p63 targeting treatments in a single composition, optionally also comprising one or more other appropriate therapeutic agent and/or anti-cancer treatments, and/or one or more pharmaceutically acceptable carries, diluents or adjuvants.

An appropriate therapeutic agent and/or anti-cancer treatments are known to one skilled in the art. Anti-cancer treatments may involve, but not limited to, a combination of treatments including but not limited to anti-cancer drugs, chemotherapy, chemotherapeutic agents, radiotherapy etc. In a variant embodiment, the anti-cancer therapy is an anti-angiogenic therapy (e.g., endostatin, angliostatin, TNP-470, capliostatin (Stachi-Fainaro et al, Cancer Cell, 7(3), 251, 2005). The therapeutic agents may be the same or different, or may be, for example, therapeutic nucleotides, drugs, hormones, hormone antagonists, receptor antagonists, enzymes or proenzymes activated by another agent, autocrines, cytokines or any other suitable anti-cancer agent known to those skilled in the art.

In connection with a anti-cancer drug which is effective against cancer, refers to the administration of an agent, such as an agent in a p73/p63 treatment, in a clinically appropriate manner results in beneficial effect for at least a statically significant fraction of subjects, such as improvement of symptoms, a cure, a reduction in disease load, reduction in tumor mass or cell numbers, extension of life, improvement in the quality of life, or other effect generally recognized as positive by medical doctors familiar with treating the particular type of disease or condition.

It will be understood that the specific dose level of a composition of the invention for any particular individual will depend upon a variety of factors including, for example, the activity of antagonists of specific p63 isoforms and/or the activity of p73/p63 targeted treatment employed, the age, body weight general health and diet of the individual to be treated, the time of administration, rate of excretion, and combination with any other treatment or therapy. Single or multiple administrations can be carried out with dose levels and pattern being selected by the treating physician.

Administration of the pharmaceutical composition is accomplished by any effective route, for example but not limited to parenterally or orally. Methods of administration include topical (for example, skin patches), inhalation, buccal, intraarterial, subcutaneous, intramedullary, intravenous, intranasal, intrarectal, intraocular administration, and other conventional means. For example, the pharmaceutical composition may be injected directly into a tumor, into the vicinity of a tumor, or into a blood vessel that supplies blood to the tumor. The pharmaceutical composition in the present invention may be administered once or more than once.

### EXAMPLES

The examples presented herein relate to the methods to identify subjects responsive to a p73/p63 targeting treatment such as, but not limited to cisplatin. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Methods.

Primary breast carcinoma specimens. Discarded tissue samples were collected under human subjects research protocol 2002-P-002059/10. All samples underwent pathological review. Microdissected tumor/normal paired samples were processed as described previously (35). For isoform-specific analysis, RNA was prepared as described below. Initially, 39 primary tumors were evaluated, and 2 were excluded due to >5% DCIS and normal epithelia.

Cell lines. The human breast carcinoma cell lines MCF-7, HCC1937, MDA-MB-468 and T47D were maintained in RPMI 1640 containing 10% FBS, 100 IU/ml penicillin and 100 μg/ml streptomycin (Invitrogen). MCF-10A cells were grown in DMEM-F12 (Invitrogen) supplemented with 5% horse serum, 20 ng/ml EGF, 0.5 μg/ml hydrocortisone, 100 ng/ml cholera toxin, 10 μg/ml insulin, 100 IU/ml penicillin and 100 μg/ml streptomycin.

Lentiviral and retroviral production and infection. The shRNA lentiviral constructs were created by transferring the U6 promoter-shRNA cassette into a lentiviral backbone, and high-titer amphotrophic retroviral and lentiviral stocks were generated by co-transfection with packaging vectors into 293T cells as described previously (14). The targeted sequences for p63 were 5′-GGGTGAGCGTGTTATTGATGCT-3′ and 5′-GAGTGGAATGACTTCAACTTT-3′. The targeted sequence for TAp73 was 5′-GGATTCCAGCATGGACGTCTT-3′.

ORT-PCR analysis. Total RNA from cells was extracted using STAT-60 RNA isolation solution (Tel-Test Inc., Friendswood, USA) according to manufacturer's protocol. First-strand cDNA was synthesized from total RNA using random hexamer primers and the SuperScript II system for RT-PCR (Invitrogen). Gene expression levels were measured by real-time QRT-PCR using the iQ SYBR Green Supermix reagent (Bio-Rad) and an OPTICON™ real-time PCR detector system (MJ Research). Data analysis was performed using OPTICON MONITOR™ Analysis Software V1.08 (MJ Research). The expression of each gene was normalized to GAPDH or B2M as a reference. The relative copy numbers were calculated from an 8-point standard curve generated from a ten fold serial dilution of full length cDNA constructs as described previously (15).

Specific forward and reverse primers (respectively) for ΔNp63 were: 5′-GGAAAACAATGCCCAGACTC-3′ and 5′-GTGGAATACGTCCAGGTGGC-3′; for TAp63 were 5′-AAGATGGTGCGACAAACAAG-3′ and 5′-AGAGAGCATCGAAGGTGGAG-3′; for TAp73 were 5′-GCACCACGTTTGAGCACCTCT-3′ and GCAGATTGAACTGGGCCATGA-3′; for ΔNp73 were 5′-CAAACGGCCCGCATGTTCCC-3′ and 5′-TTGAACTGGGCCGTGGCGAG-3′; for GAPDH were: 5′-CACCCAGAAGACTGTGGATGG-3′ and 5′-GTCTACATGGCAACTGTGAGG-3′ and for detection of B2M were 5′-AGCTGTGCTCGCGCTACTCTC-3′ and 5′-CACACGGCAGGCATACTCATC-3′. The conditions for all QRT-PCR reactions were: 3 min at 94° C., followed by 40 sec at 94° C., 40 sec at 60° C. and 25 sec at 72° C. for 40 cycles. All PCR products were confirmed by the presence of a single-peak upon melting-curve analysis and by gel electrophoresis. No-template (water) reaction mixtures and “no reverse transcriptase” mixtures were performed on all samples as negative controls. All experiments were performed in duplicate.

Apoptosis assays. Apoptotic assays were performed as described previously (14). Briefly, both floating and attached cells were collected 72 hours after p63-directed shRNA lentiviral infection. Apoptotic cell death was determined using the BD ApoAlert annexin V-FITC Apoptosis Kit (BD Biosciences) according to the manufacturer's instructions, and cells were analyzed on a FACSCalibur flow cytometer using CellQuest Pro software (BD Biosciences).

Immunoprecipitation (IP) and immunoblot analysis. Protein lysates from cells were extracted in ice-cold lysis buffer (0.75% NP-40; 1 mM DTT; protease inhibitors in PBS) with/without phosphates inhibitor I and II (Sigma). For IP experiments, pre-cleared lysates (2.5 mg) were incubated with either 2.0 μg of anti-p63 polyclonal antibody (H-129, Santa Cruz) or 1.0 μg of anti-p73 monoclonal antibody (Ab-2, CalBiochem) for 2 hours at 4° C. The immunocomplexes were precipitated using protein A or protein G Sepharose (Amersham Biosciences), washed four times with lysis buffer and analyzed by SDS-PAGE. Immunoblots were probed with the following antibodies: p63 (1:5000; 4A4, Sigma-Aldrich); mouse monoclonal p73 (1:1000; Ab-2, CalBiochem); PARP (1:1000; Cell Signaling Technology); PUMA (1:1000; Ab9645, Abcam); β-tubulin (1:2500; D-10, Santa Cruz Biotechnology); NOXA (1:1000; Ab13654, Abcam); phosho-Tyr (1:1000; PY99, Santa Cruz Biotechnology) and p300 (1:1000; N-15, Santa Cruz Biotechnology).

Chemosensitivity assay. Dose-response curves and IC₅₀ values were determined using the methyl thiazolyl tetrazolium (MTT) cell viability assay as described previously (52). Cells were seeded into 96-well microtiter plates for 24 hours at a density of 5×10³ per well. Serial drug dilutions were prepared in medium immediately before each assay and viable cell masses following 3 days or 5 days of drug exposure were determined by cell-mediated MTT reduction. Cell growth as well as drug activity were determined by measuring absorbance at 550 nm using an Anthos Labtec systems plate reader.

Statistical Methods. For continuous variables, unpaired, two-way Student's t-tests are used to evaluate differences between sample means, and all values are reported as mean±s.e.m. No significant deviations from normality were observed. ΔNp63 and TAp73 levels greater than two times the sample mean for each gene (n=37) were considered elevated. In order to ensure that our conclusions were robust to small sample sizes, 2-tailed Fisher's Exact tests were used to derive p-values from the 2×2 contingency tables. Multiple comparisons were not carried out except as indicated, and thus no corresponding compensation was made. In all cases, an alpha level of 0.05 was taken to indicate significance.

### Example 1

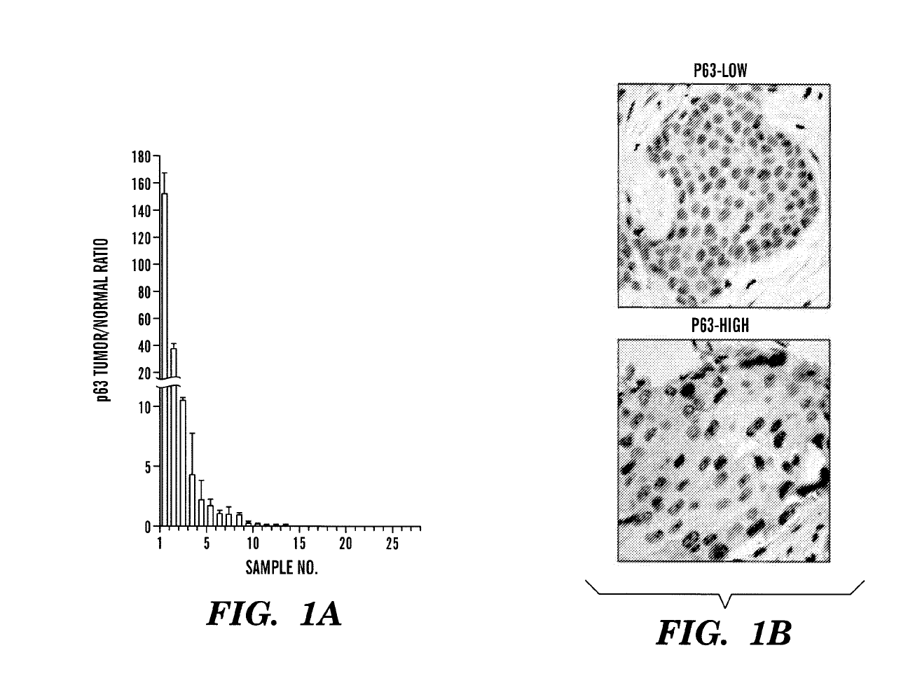

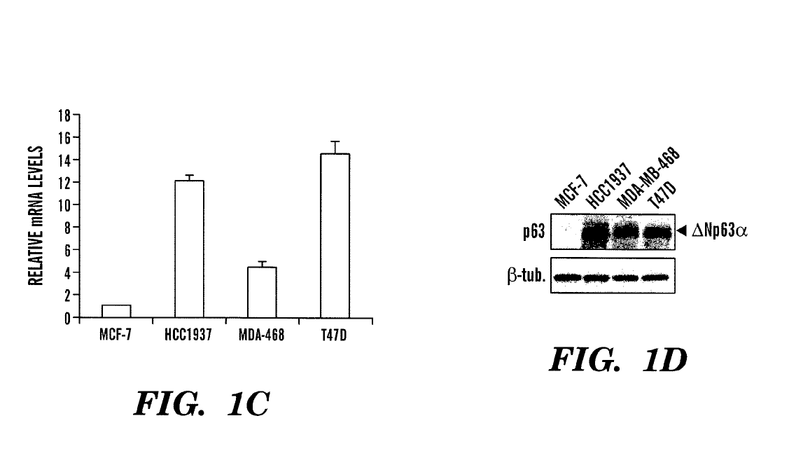

p63 is expressed in a minority of primary invasive breast cancers and tumor-derived cell lines. Previous studies of p63 expression in breast carcinoma have relied almost exclusively on immunohistochemistry, which is relatively insensitive and only roughly quantitative. In order to perform quantitative assessment of p63 levels we used a panel of breast tumor specimens that have been extensively characterized by laser-capture microdissection (LCM) and microarray-based gene expression profiling (35). These specimens allowed us to compare p63 expression levels in specimen-matched normal luminal epithelial cells and invasive carcinoma cells. The inventors directly examined p63 expression by real-time quantitative RT-PCR (QRT-PCR) in 27 matched normal epithelium/invasive tumor pairs. While most tumors exhibited equal or lower levels of p63 expression than matched normals, p63 was found to be expressed at more than 2-fold the matched normal level in 4 of 27 specimens (FIG. 1A). The inventors then asked whether p63 was also overexpressed in the pre-malignant ductal carcinoma in situ (DCIS) component of these same specimens, which had been microdissected separately from luminal and invasive carcinoma cells (35). In total, p63 expression was examined in 39 pairs of matched DCIS and normal specimens. Only 1 of these 39 cases demonstrated significantly higher p63 expression in DCIS cells than in normal epithelium (data not shown), indicating p63 is expressed in a subset of primary breast carcinomas, and that its expression may be restricted largely to invasive disease.

To confirm that these results reflected p63 expression within malignant cells immunohistochemistry was performed for p63 in specimens exhibiting both low and high p63 levels. Consistent with the results from QRT-PCR, nuclear expression of p63 protein was observed specifically in malignant epithelial cells from cases showing p63 mRNA overexpression, and not from cases showing low/no expression (FIG. 1B). Thus p63 mRNA and protein are expressed in a subset of primary breast tumor cells.

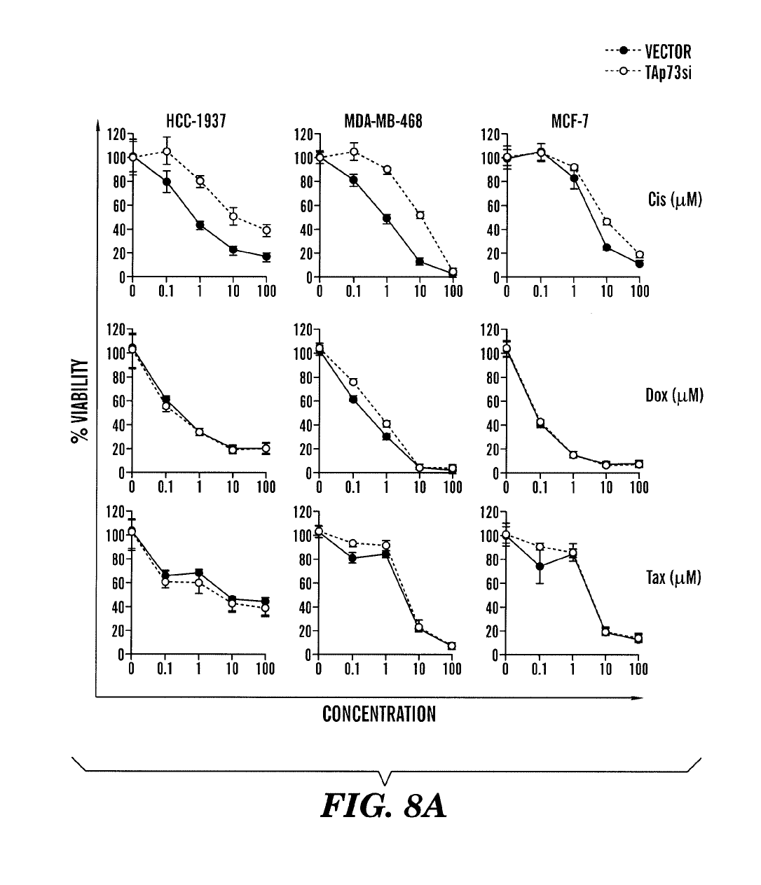

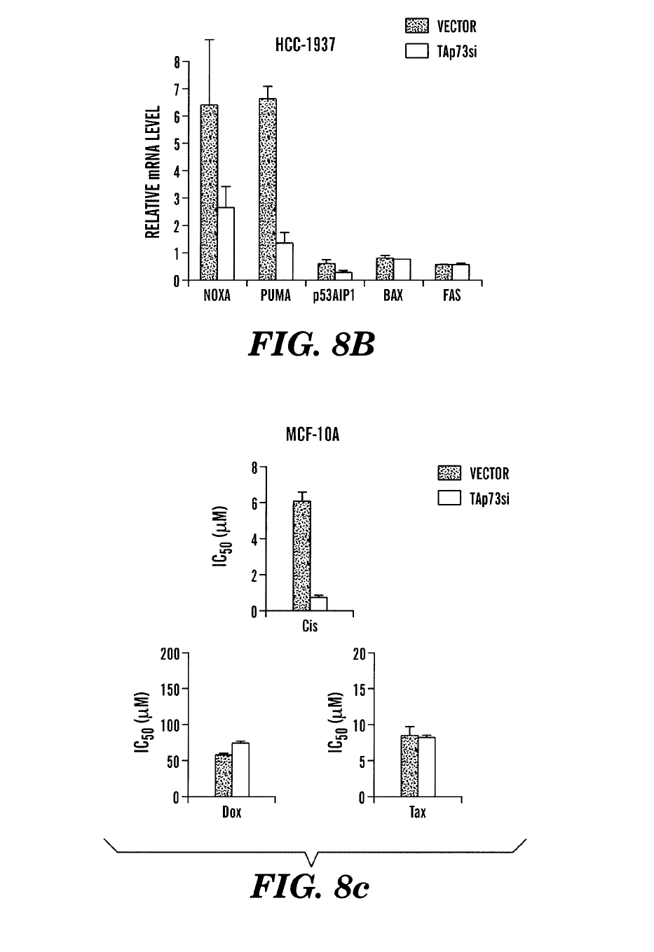

The inventors next examined p63 expression in human tumor-derived breast carcinoma cell lines. Consistent with the findings in primary breast tumors, the inventors found that p63 mRNA and protein are expressed in a subset of breast cancer cell lines. Several cell lines including MCF-7 exhibit low levels p63 expression. In contrast, other lines including HCC-1937, T47D and MDA MB-468 express significant levels of p63 (FIG. 1C and data not shown). Using a combination of isoform-specific QRT-PCR and western analysis the inventors found that all three of these cell lines express predominantly ΔNp63α, which is the major p63 isoform expressed in normal epithelia (FIG. 1C, D). TAp63 mRNA is less abundant, and TAp63 protein isoforms are undetectable by western analysis in these cells (data not shown). Predominant expression of ΔNp63 rather than TAp63 is further substantiated by data presented in FIG. 8, showing robust expression of ΔNp63 but not TAp63 in a subset of primary breast tumors. Thus, ΔNp63α is the major p63 isoform expressed in both primary breast tumors and cell lines.

### Example 2

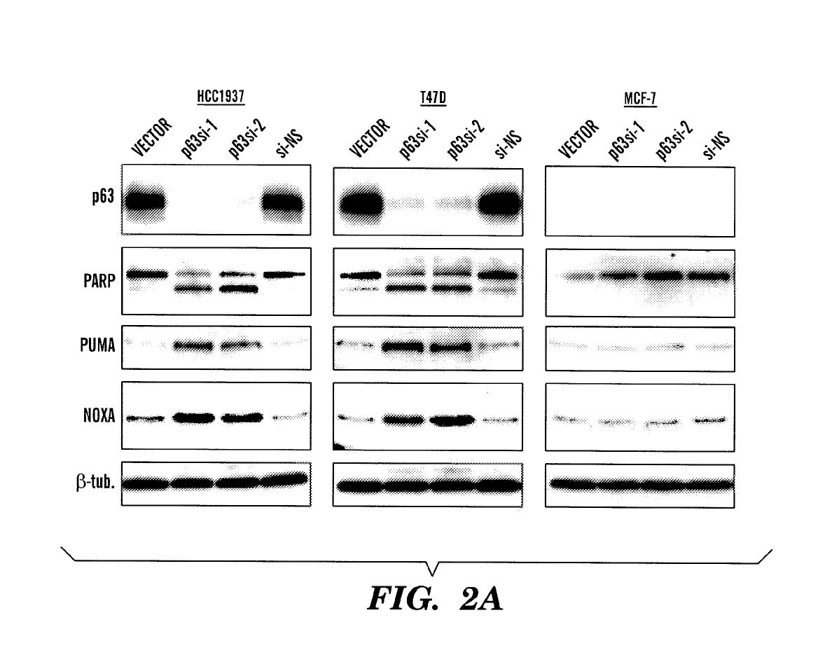

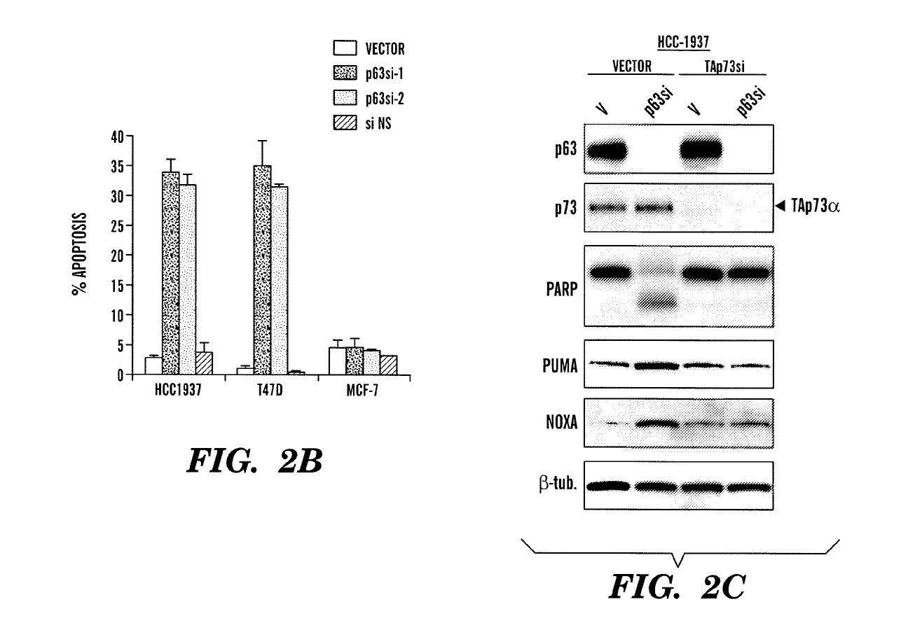

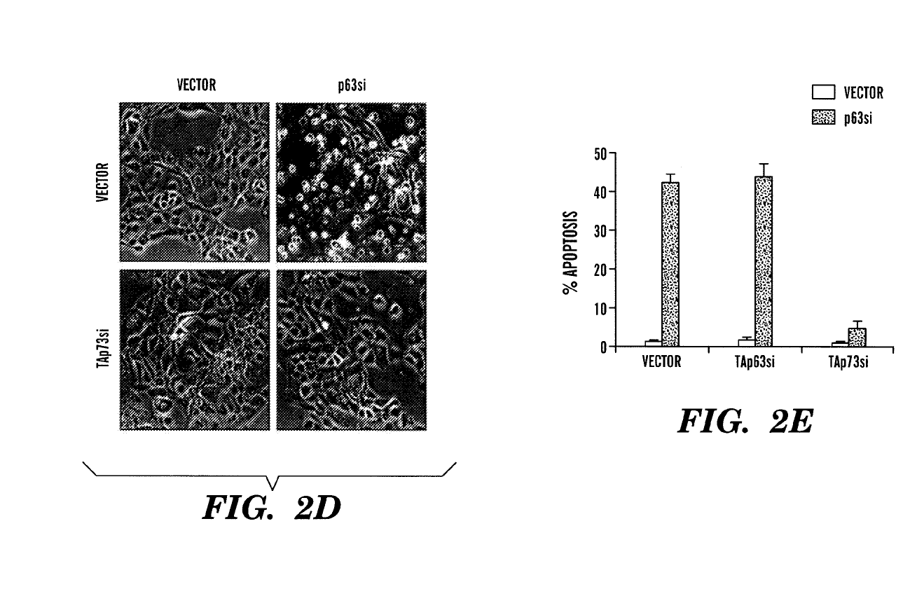

p63 is required for survival in breast cancer cells. The inventors developed an efficient system to study the function of endogenous p63 using lentiviral-based RNA interference (RNAi). These viruses express small hairpin RNA (shRNA) species that are processed to small inhibitory RNAs (siRNAs). The inventors tested the effect of endogenous p63 knockdown by lentiviral RNAi in breast cancer cells that express endogenous ΔNp63α. As a control for the specificity of the shRNA species, the effect of two independent p63-directed shRNAs were also tested. As an additional control, the effects of expressing these shRNAs in MCF-7 and SAOS-2 cells, both of which express little or no endogenous p63 were also examined (FIG. 2A).

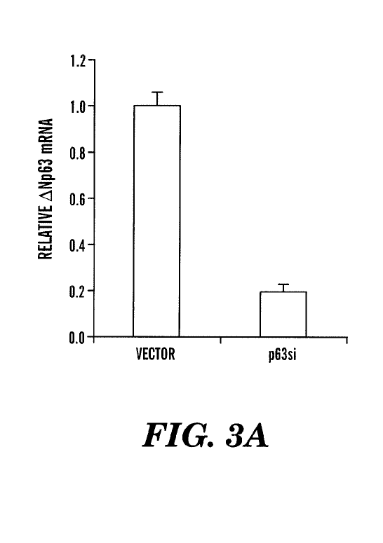

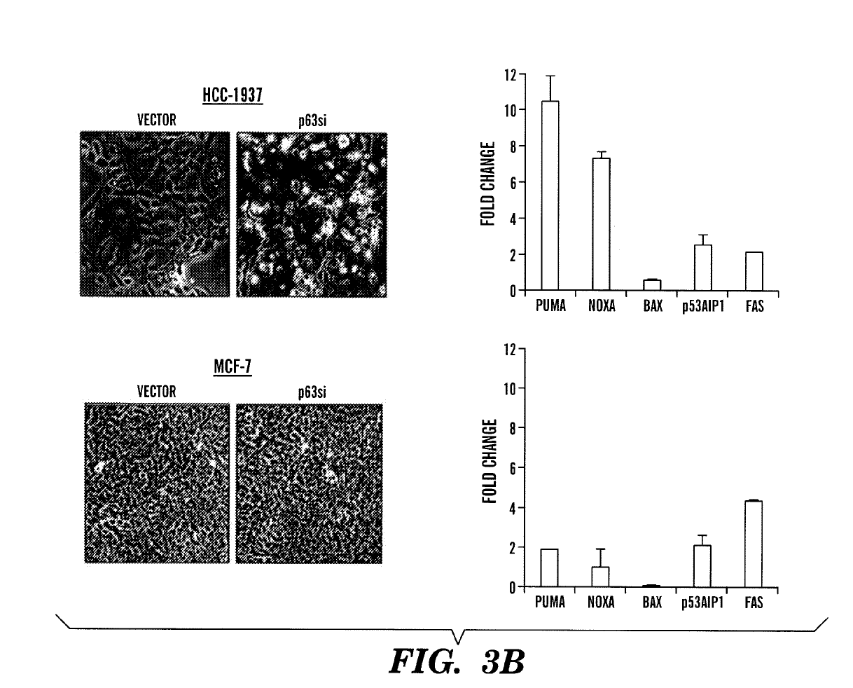

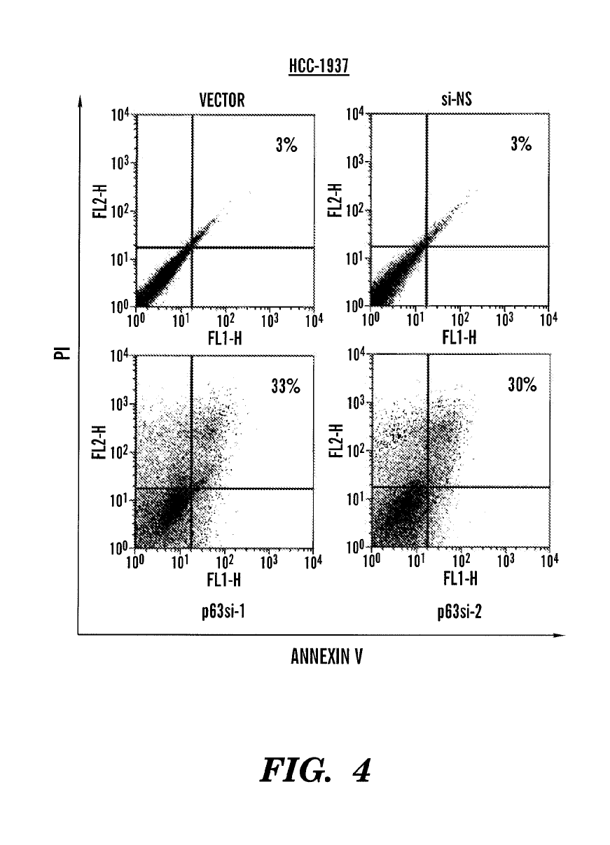

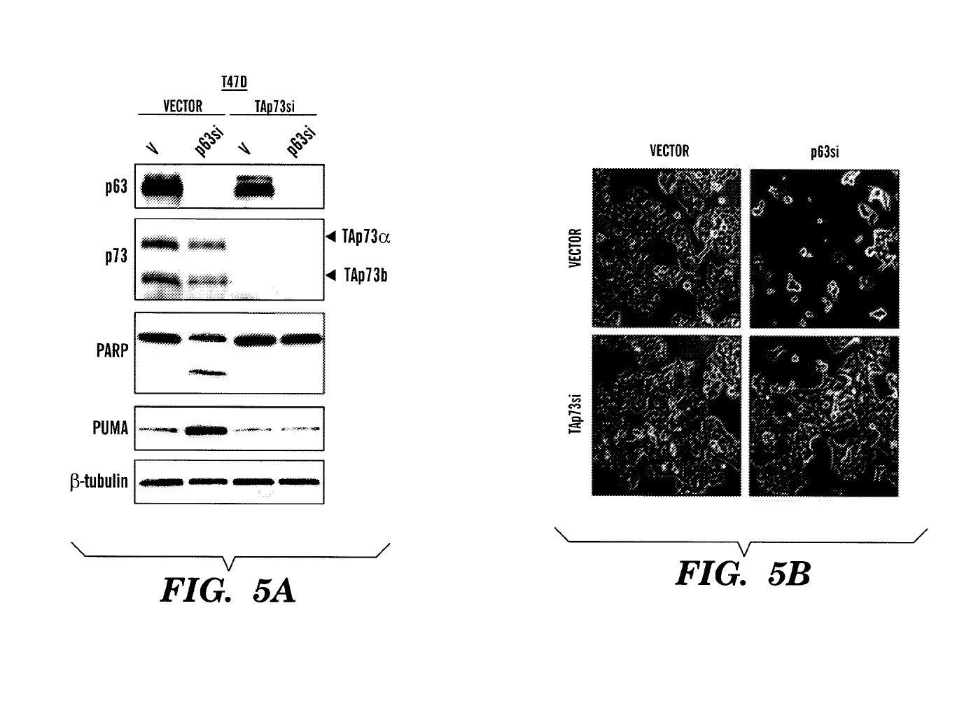

p63 expression is efficiently reduced by lentiviral RNAi in HCC-1937 and T47D cells, both of which express endogenous ΔNp63α. The inventors routinely achieve ≧75% knockdown of p63 mRNA and protein within 48-72 hours of lentiviral infection in these cells, as assessed by QRT-PCR and western analysis, respectively (FIG. 2A and FIG. 3). In contrast, infection with the control lentivirus or a non-specific shRNA does not affect endogenous p63 levels. Inhibition of p63 in both these p63-expressing breast cancer lines induced obvious cell death associated with nuclear blebbing characteristic of apoptosis (FIG. 2D). These morphologic changes were not observed following infection of these cells with either control lentivirus, nor were they observed following p63-directed RNAi in either MCF-7 or Saos-2 cells (FIG. 3 and (14)).

To determine whether p63 loss induced apoptosis, western analysis was performed for cleavage of poly(ADP-ribose) polymerase-1 (PARP-1), a specific hallmark of apoptotic cell death. PARP-1 cleavage was observed specifically in response to p63 knockdown and not following control lentiviral infection, and it was observed only in HCC-1937 and T47D cells, which express endogenous ΔNp63α (FIG. 2A). To quantitate apoptotic cell death, the inventors assayed annexinV/propidium iodide (PI) staining of unfixed cells by FACS analysis. This assay detects both early apoptotic (annexin V positive/PI negative) and late apoptotic (annexin V positive/PI positive) cells (36). Prominent annexin V/PI staining correlated with PARP-1 cleavage and with morphologic features of apoptosis, and was induced specifically in response to p63 knockdown only in cell lines expressing endogenous p63 (FIGS. 2A, 2B and FIG. 3). Therefore, specific inhibition of endogenous p63 induces apoptosis in breast cancer cells in which it is expressed.

To begin to address the mechanism by which p63 inhibition induces apoptosis of breast cancer cells, the inventors first assayed expression of pro-apoptotic genes known to undergo regulation by p53 family members. A significant induction of PUMA and NOXA, but not Bax, AIP1, or Fas was observed, coincident with p63 knockdown in HCC-1937 and T47D cells (FIG. 2A and FIG. 3). Induction of both PUMA and NOXA was a specific effect of p63 inhibition, as neither gene was induced following control shRNA lentiviral infection in these cells, nor were they induced by p63-directed shRNA in MCF-7 or Saos-2 cells, which do not express endogenous ΔNp63α (FIG. 2A, FIG. 3).

### Example 3

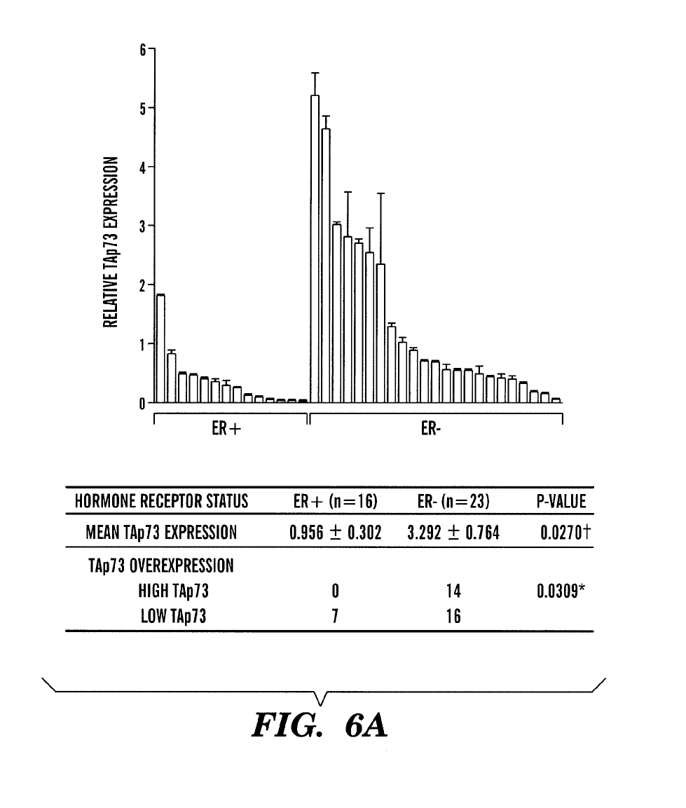

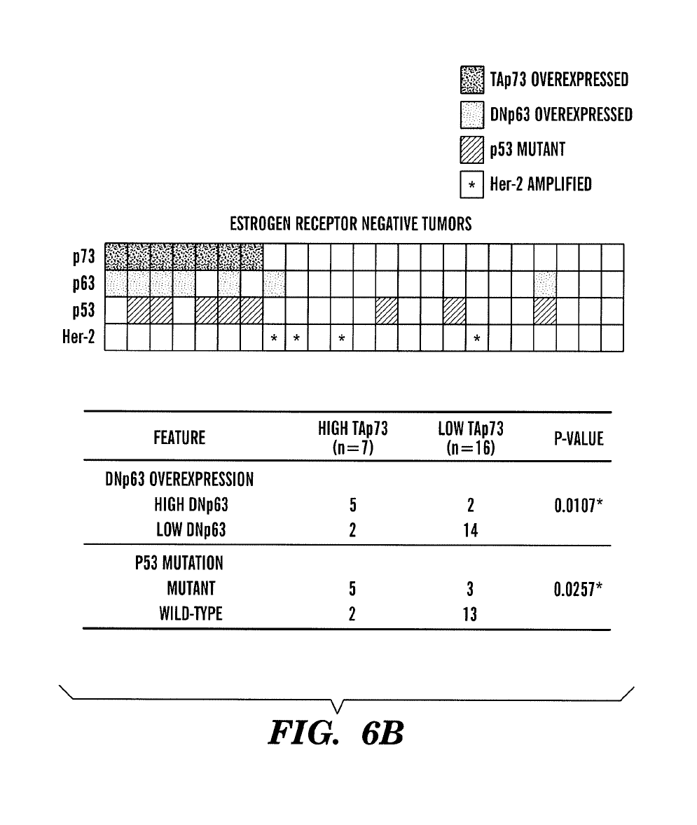

Apoptosis following p63 inhibition depends on endogenous p73. DNp63a has been hypothesized to function as an inhibitor of its pro-apoptotic paralogues p53 and TAp73 (13, 20, 21, 35). Neither HCC-1937 nor T47D cells express wild-type p53, demonstrating that the p63-dependent survival effect in these cells is p53 independent (36). In contrast, both cell lines express TAp73 isoforms (FIGS. 6A, 6D), and the inventors and others have previously demonstrated that TAp73 is a direct transcriptional activator of PUMA, implying a potential contribution of TAp73 to induction of PUMA and cell death following inhibition of p63 (37, 38). In order to determine whether PUMA induction and apoptosis following loss of p63 were mediated by TAp73, the inventors inhibited TAp73 in these cells by expressing a lentiviral shRNA construct that is a potent and specific inhibitor of this isoform, followed by brief drug selection (37). Knockdown of p73 mRNA and protein was confirmed by QRT-PCR and immunoprecipitation (IP)/western analysis, respectively, in these cells compared to control vector-infected cells (FIG. 6A). Cell death following p63 knockdown in the TAp73-inhibited cells was quantitated as compared to control cells. As an additional control, cell death was quantitated in cells expressing a shRNA directed against TAp63, which is expressed at very low levels in these cells (FIG. 1). Only inhibition of TAp73 consistently abrogated PUMA induction, PARP cleavage, and cell death following knockdown of DNp63a (FIG. 6A, B). [Similar results were observed in T47D and MDA MB-468 cells]. Therefore, DNp63a, when present in breast cancer cells, promotes cellular survival by inhibiting the pro-apoptotic activity of transactivating isoforms of p73.

In squamous carcinoma cells endogenous DNp63a binds TAp73 to inhibit its transcriptional activity (13). To determine whether this same mechanism was operative in breast carcinoma cells, we assayed for DNp63a/TAp73 interaction by co-immunoprecipitation of the respective endogenous proteins. In both HCC-1937 and T47D cells endogenous TAp73 was immunoprecipitated by p63-directed antisera, and endogenous DNp63a was immunoprecipitated following IP for p73 (FIG. 6D). [Controls that demonstrate a lack of cross reactivity between these antibodies have been shown previously (13)]. In both cell lines DNp63a is in molar excess relative to TAp73, and in both lines immunodepletion studies suggest that the vast majority of TAp73 is complexed to DNp63a (FIG. 6D). Therefore, DNp63a functions to suppress TAp73 pro-apoptotic activity in breast cancer cells through direct physical interaction.

### Example 4

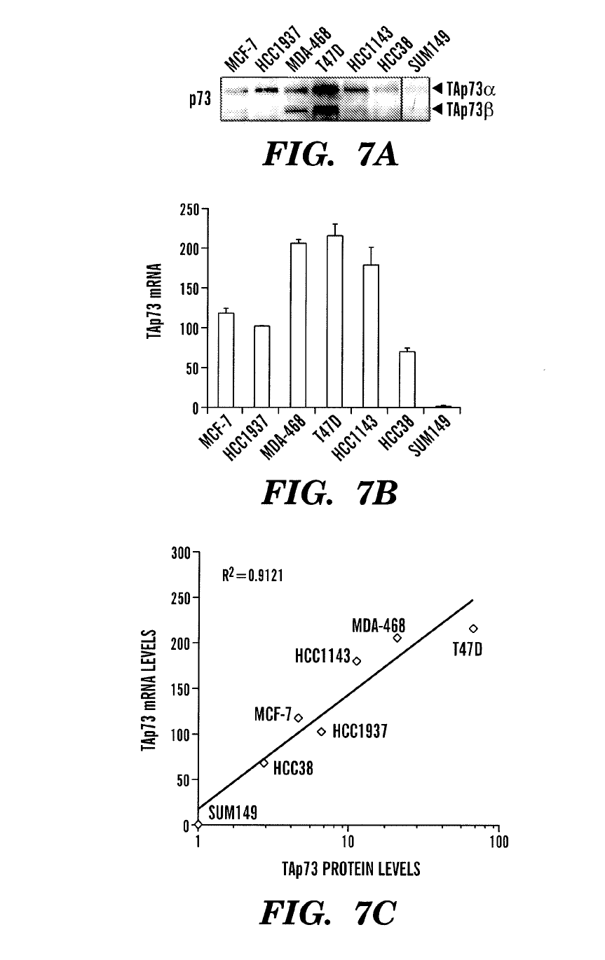

Co-expression of TAp73 and ΔNp63 in a biologically defined subset of primary invasive breast carcinomas. Given the findings above, the inventors assayed the expression of specific p63 and p73 isoforms in a quantitative manner in primary breast tumors. The inventors identified a group of 39 primary invasive breast carcinoma specimens from which frozen tissue was available for RNA preparation and analysis. Of note, all tumors were removed prior to any radiation, hormonal or chemotherapy. Isoform-specific QRT-PCR was used to directly assess levels of ΔNp63 and TAp73 in these samples. This was validated by comparing mRNA levels assessed by QRT-PCR to the respective protein levels determined by western analysis in a panel of breast cancer cell lines. The inventors obtained a correlation (R²) value of >0.9 for both TAp73 and ΔNp63 (FIG. 7). The inventors also assayed other isoforms, including TAp63 and N-terminally truncated p73 isoforms (ΔNp73 and DN′p73, hereafter referred to as ΔNp73) in these samples. In order to compare the level of each isoform relative to the others, cDNA templates were used to generate standard curves for each isoform (see methods). To ensure the findings reflected gene expression within malignant cells, samples underwent histopathologic analysis and were found to contain no more than 5% DCIS or normal epithelia.

Examining a series of 39 such tumor specimens, TAp73 was overexpressed (more than 2 fold the mean value of the sample set) in a subset of breast carcinomas lacking estrogen receptor (ER) and progesterone receptor (PR) expression (FIG. 6A). Overexpression of TAp73 was not observed in ER-positive tumors, a finding that was statistically significant (FIG. 6A). In contrast, ΔNp73 isoforms were variably expressed but in all cases were more than 10-fold less abundant than TAp73 isoforms. Low abundance of ΔNp73 relative to TAp73 isoforms is supported by two recent reports demonstrating an excess of TAp73 versus ΔNp73 isoforms of 8 to 100-fold in primary breast and ovarian carcinomas (33, 40).

The overexpression of ΔNp63 was highly correlated with TAp73 overexpression in these tumors (FIG. 6B). This observation is consistent with the finding that ΔNp63 is required for survival in tumor cells with high TAp73 expression. In contrast, TAp63 isoforms were expressed at significantly lower levels than ΔNp63 in most of the specimens and were not associated with TAp73 expression. Given that p63 and p73 may also exhibit functional interactions with p53, we next examined the mutational status of p53 in these tumors by cDNA sequencing (Table 1). Among the seven tumors exhibiting overexpressed TAp73, five of seven harbored missense or nonsense mutations in p53, while only 3 of 16 tumors lacking TAp73 overexpression exhibited mutant p53 (FIG. 6B). This finding was statistically significant and was also in agreement with a prior report suggesting as association between expression of p73 and mutant p53 in primary breast carcinomas (FIG. 6B) (41).

As noted above, breast tumors characterized by the absence of both ER/PR expression and Her-2 overexpression appear biologically and clinically distinct (1, 16). The inventors assessed Her-2 mRNA expression in the tumor set by QRT-PCR, using a clinically validated cutoff value for Her-2 overexpression of greater than 10-fold the mean normal level (42). No tumors were found to exhibit co-overexpression of TAp73 and ΔNp63 showed Her-2 amplification (FIG. 6B). Therefore, overexpression of TAp73 and ΔNp63 is restricted to a subset of ER/PR/Her-2 negative (so-called “triple-negative”) primary breast carcinomas that most commonly exhibit mutation of p53. Consistent with these findings, two of three tumor-derived cell lines we identified as co-expressing ΔNp63 and TAp73, HCC-1937 and MDA MB-468, exhibit a triple negative phenotype and mutational inactivation of p53. These tumors may therefore represent a distinct biologic subtype with particular phenotypic properties.

### Example 5

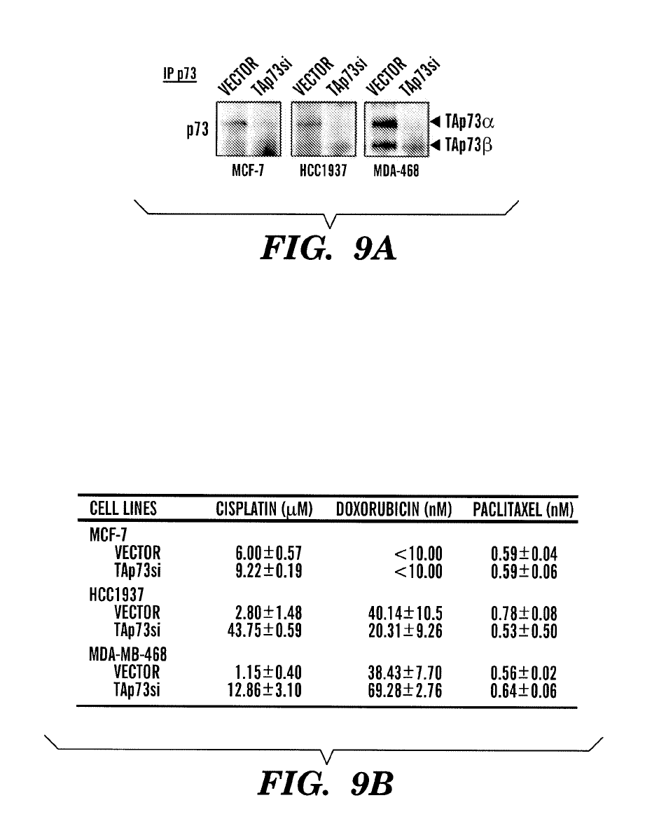

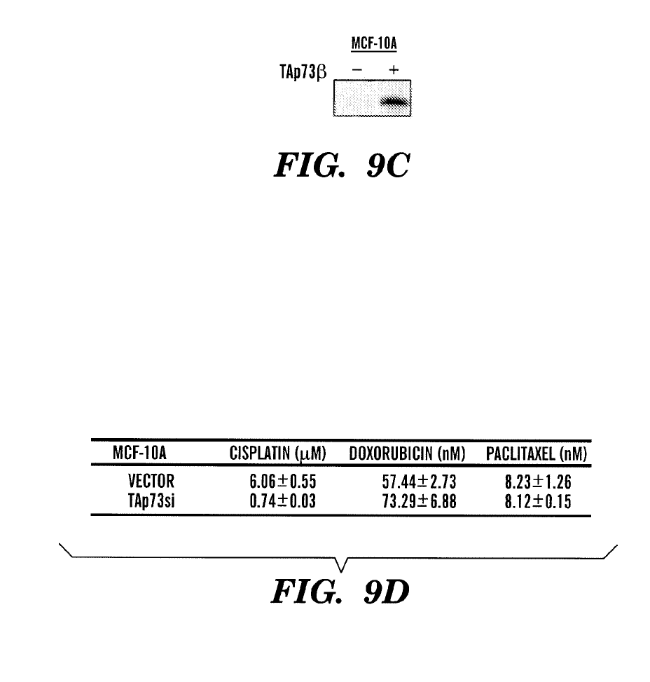

TAp73 mediates specific cisplatin sensitivity in breast cancer cells exhibiting ΔNp63/TAp73 expression. Expression of p73 has recently been demonstrated to contribute to chemosensitivity of tumor cells in a variety of contexts (25-29). Therefore, whether the unique expression of ΔNp63 and TAp73 in triple-negative breast cancer cells might mediate particular chemosensitivity was assessed. To directly assess the contribution of TAp73, the inventors tested the effect on chemosensitivity of ablating endogenous TAp73 by lentiviral RNAi in HCC-1937 and MDA MB-468, two triple negative cell lines that express ΔNp63 and TAp73. Pools of cells stably expressing the TAp73-directed lentivirus or the vector control were generated. As a control, the cell line MCF-7, an ER-positive, p63-negative breast cancer line that is not p63-dependent was also examined (FIG. 2). The inventors first tested chemosensitivity to doxorubicin or paclitaxel, two of the most commonly used agents for treatment of early-stage breast cancer (43). Knockdown of TAp73 had less than a 2-fold effect on the IC₅₀ (50% inhibitory concentration) for either drug using a quantitative cell viability assay (FIG. 8A). In contrast, TAp73 ablation induced marked cisplatin resistance in both triple-negative cell lines: the IC₅₀ for cisplatin was increased by more than 10 fold in each of these cell lines following TAp73 inhibition (FIG. 9). In MCF-7 cells TAp73 was not a mediator of sensitivity to cisplatin or other chemotherapeutic agents (FIG. 8A). Therefore, TAp73 is a mediator of cisplatin sensitivity in breast cancer cells expressing ΔNp63 and TAp73.

TABLE 1
Mutational status was determined based in bi-directional
sequencing (exon 1-7), and was verified by the presence of
nucleotide changes in both strands.
p53 Status in Clinical Breast Cancer Samples

| | | Codon 72 | | Codon |
|---|---|---|---|---|
| Sample ID | P53 Status | polymorphism | Codon nucleotide | Amino Acid |
| 715 | Mutant | Pro | 577 (C to T) | H193Y |
| 1273 | Mutant | Pro | 817 (C to T) | R273C |
| 1325 | WT | Pro | | |
| 1502 | WT | Arg | | |
| 1106 | WT | Arg | | |
| 1322 | WT | Arg | | |
| 999 | WT | Arg | | |
| 1414 | WT | Pro | | |
| 400 | WT | Arg | | |
| 1274 | Mutant | Pro | 151 (G to T) | Stop |
| | | | 152 (A to G) | E51G |
| 1509 | WT | Pro | | |
| 465 | Mutant | Arg | Del (529-546) | |
| 1196 | Mutant | Pro | 730 (G to A) | G244S |
| 209 | Mutant | Arg | 818 (G to A) | R273H |
| 1321 | WT | Arg | | |
| 588 | WT | Unknown | | |
| 164 | Mutant | Arg | 523 (C to G) | R175G |
| 720 | WT | Arg | 639 (A to G) | R213R |
| 158 | WT | Arg | | |
| 222 | WT | Unknown | | |
| 1278 | WT | Arg | | |
| 756 | WT | Arg | | |
| 489 | Mutant | Pro | 469 (G to T) | V157F |
| SN3 | WT | Pro | | |
| 175H | Mutant | Arg | 524 (G to A) | R175H |
| 277Y | Mutant | Pro | 830 (G to A) | C277Y |
| 481 | WT | Pro | | |
| 488 | WT | Pro | | |
| 432 | WT | Pro | | |
| 282 | WT | Pro | | |
| 484 | WT | Pro | | |
| 242 | WT | Pro | | |
| 477 | WT | Pro | | |
| 480 | WT | Pro | | |
| 398 | WT | Pro | | |
| 298 | WT | Pro | | |
| 213 | WT | Arg | | |
| 469 | WT | Pro | | |
| 426 | WT | Pro | | |
| 427 | WT | Pro | | |
| 399 | WT | Pro | | |
| 430 | WT | Pro | | |
| 436 | WT | Pro | | |
| 289 | WT | Pro | | |
| 253 | WT | Pro | | |
| 356 | WT | Pro | | |

To further explore the p73-dependent pathway induced by cisplatin, the inventors next examined expression of candidate apoptotic effector genes following chemotherapy treatment. A pattern of gene induction was observed that was remarkably similar to that observed following p63 knockdown: PUMA and NOXA, but not other pro-apoptotic effector genes, were robustly induced following cisplatin treatment. Most importantly, their induction was p73-dependent as it was attenuated following treatment of TAp73-directed shRNA (FIG. 8B). In contrast, doxorubicin treatment resulted in only a 2-fold increase in PUMA and no significant induction of other pro-apoptotic effectors at an equally toxic concentration. Therefore, TAp73 induces a transcriptional program specifically in response to cisplatin treatment that leads to the death of breast cancer cells.

In a previous report, the inventors reported that unlike breast cancer cells, normal basal mammary epithelial cells express ΔNp63α but little or no TAp73 (8). The findings herein predict that ectopic TAp73 expression in such normal cells should enhance cellular sensitivity to cisplatin. To test this possibility, the inventors expressed TAp73 ectopically in MCF-10A, a non-transformed, non-tumorigenic breast epithelial cell line that expresses relatively high levels of ΔNp63α but little or no TAp73 (8). MCF-10A cells were stably infected with a retrovirus encoding TAp73β, followed by brief drug selection (FIG. 9). Ectopic TAp73β expression significantly increased the sensitivity of MCF-10A cells to cisplatin (FIG. 9), even though it did not affect their baseline cell proliferation or viability. This effect was highly specific, as TAp73β expression did not significantly enhance sensitivity to either doxorubicin or paclitaxel (FIG. 8C). Therefore, TAp73, which is normally inactive in breast cancer cells due to co-expression of ΔNp63α, is activated to induce a cell death pathway specifically in response to cisplatin treatment, resulting in chemosensitivity to this agent.

### Example 6

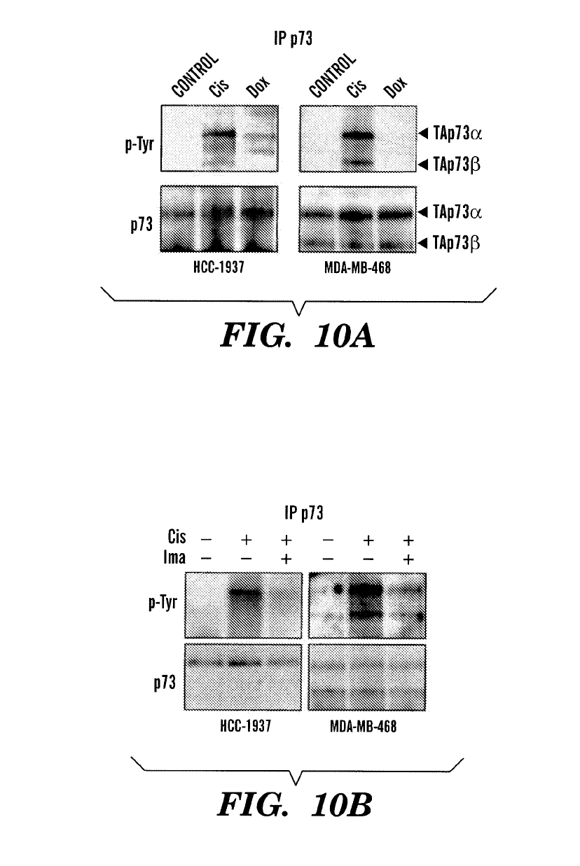

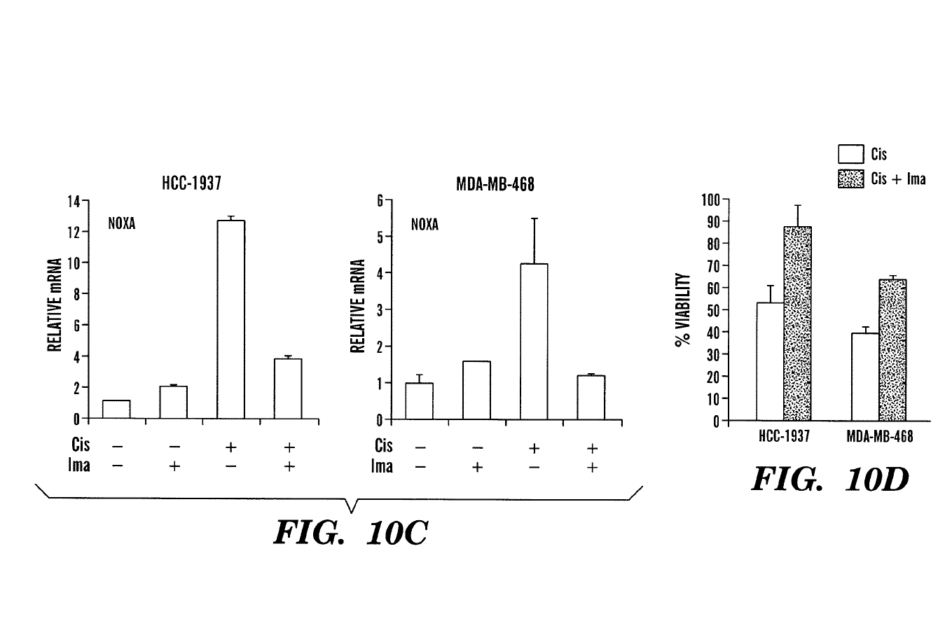

Phosphorylation-dependent activation of TAp73 by cisplatin. The inventors next investigated the mechanism by which cisplatin treatment induced specific p73 activation in breast carcinoma cells. TAp73-dependent transcription of PUMA and NOXA was observed within 2 hours of cisplatin treatment, suggesting a rapid mechanism of p73 activation. Previous studies have implicated c-Abl-dependent phosphorylation in the activation of p73 following DNA damage (27-29). Therefore, the inventors tested whether p73 was tyrosine phosphorylated following chemotherapy treatment. In both HCC-1937 and MB-468 tyrosine phosphorylation of p73 was observed, which is maximal at 6 hours, specifically in response to cisplatin but not doxorubicin treatment (FIG. 10A). To determine whether phosphorylation was c-Abl-dependent the inventors used the well-established c-Abl kinase inhibitor STI-571 (44). Pretreatment of cells with STI-571 resulted in substantial inhibition of p73 phosphorylation following cisplatin treatment (FIG. 10B).

The inventors then sought to address the functional role of c-Abl-dependent phosphorylation of p73 in the cisplatin-induced response. Pretreatment with STI-571 blocked p73-dependent induction of NOXA following cisplatin treatment in both HCC-1937 and MB-468 cells, even though it had no effect on baseline NOXA expression in either cell line (FIG. 10C). Most importantly, the ability of STI-571 to block p73 phosphorylation and p73-dependent pro-apoptotic transcription correlated with a substantial rescue from cell death induced by cisplatin in both HCC-1937 and MB-468 cells (FIG. 10D). Of note, STI-571 treatment did not affect cell proliferation in either cell line at the dose used in these assays. Therefore, TAp73 is phosphorylated in a c-Abl-dependent manner in breast cancer cells specifically in response to cisplatin treatment, and that this phosphorylation is essential for cisplatin-induced apoptosis.

### Example 7

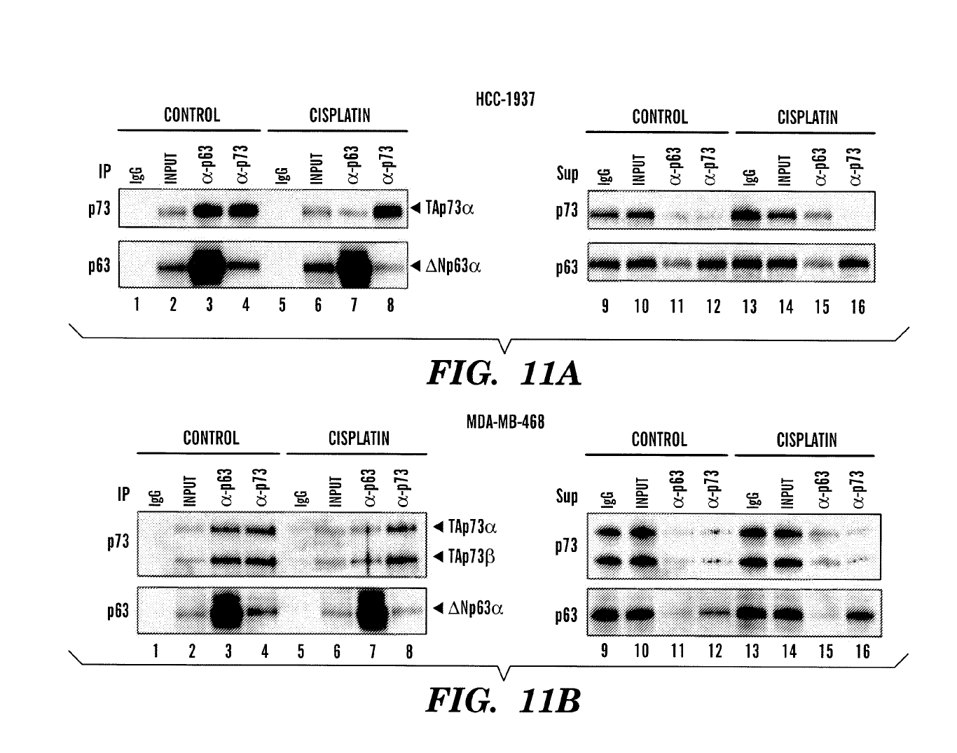

Dissociation of the p73/p63 complex following c-Abl-dependent phosphorylation. In squamous carcinomas endogenous ΔNp63α is bound to TAp73, which the inventors and others have shown to be sufficient for repression of TAp73-dependent transcription (14, 37). To determine whether this same mechanism was operative in breast carcinoma cells, the inventors examined ΔNp63α/TAp73 interaction by co-immunoprecipitation of the respective endogenous proteins. In both HCC-1937 and MDA-468 cells endogenous TAp73 was immunoprecipitated by p63-directed antisera, and similarly endogenous ΔNp63α was immunoprecipitated following IP for p73 (FIG. 11A). Furthermore, in both cell lines ΔNp63α is in molar excess relative to TAp73, and each of the IP for either p63 or p73 yielded the same mass of p73 and results in essentially complete depletion of p73 from the lysate (FIG. 11A). Therefore the vast majority of TAp73 is complexed to ΔNp63α in breast cancer cells under normal growth conditions.

As noted above, TAp73-dependent transcription of pro-apoptotic effector genes was observed within several hours of cisplatin treatment, in the absence of any change in ΔNp63α or TAp73 protein levels (FIGS. 8B, 10C, 11). The inventors therefore investigated how cisplatin treatment allows TAp73 to escape ΔNp63α-mediated repression. The inventors observed a substantial dissociation of the ΔNp63α/TAp73 complex following cisplatin treatment, as assessed by co-IP for either endogenous protein (FIG. 11). These findings were confirmed by examining the immunodepleted lysates: little or no residual p73 is detectable following depletion for p63 in the basal state; in contrast “free” TAp73 is readily detectable following cisplatin treatment (FIG. 11). While we do observe a decline in ΔNp63α protein expression beginning at 12 hours consistent with prior reports (45), our findings suggest that dissociation of the ΔNp63/TAp73 complex contributes to cisplatin-induced TAp73 activation.

Finally, the inventors asked whether the requirement for c-Abl-dependent phosphorylation in the cisplatin response might be explained by its ability to promote dissociation of the ΔNp63α/TAp73 complex. Consistent with this hypothesis, pre-treatment of cells with STI-571 substantially blocked dissociation of this complex at 6 hours, which correlated with its ability to block p73-dependent transcription at this same time point (FIGS. 11, 10C). Therefore activation of TAp73 in response to cisplatin treatment involves c-Abl-dependent phosphorylation, which promotes TAp73 dissociation and consequent escape from ΔNp63α-mediated repression, ultimately resulting in tumor cell death.

### Example 8

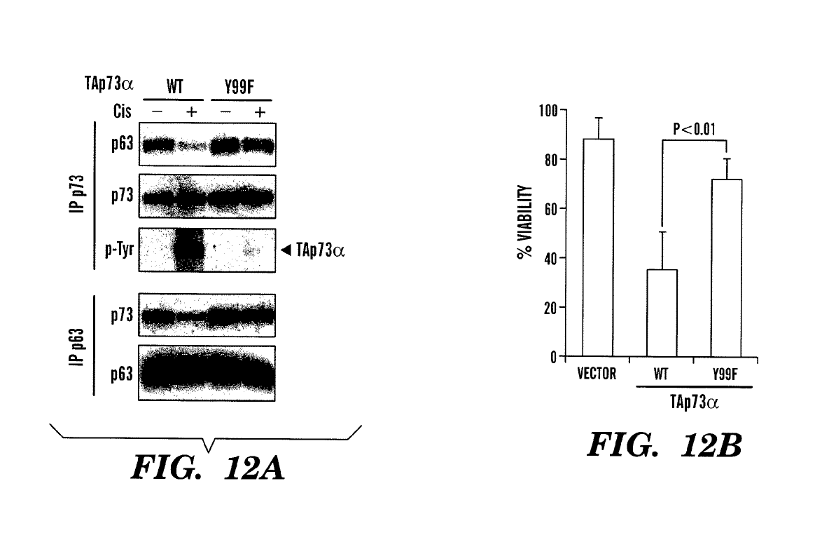

Phosphorylation of TAp73 Y99 is essential for dissociation of the p73/p63 complex and for cisplatin sensitivity. The inventors investigated whether c-Abl-dependent phosphorylation of p73 was required for p73/p63 dissociation and for the specific chemosensitivity to cisplatin mediated by TAp73. Previously published data indicate that Y99 is the major site of p73 phosphorylation by c-Abl and that phosphorylation at this site is critical for activation of p73 in response to DNA damage. Therefore, the inventors compared the effects of expressing either wild-type TAp73α or the site-specific Y99F mutant TAp73α in MCF-10A cells. Of note, these cells express endogenous ΔNp63α but not TAp73. The inventors discovered that stable retroviral expression of TAp73 did not affect baseline proliferation or viability of MCF-10A cells (data not shown). The inventors discovered that ectopic wild-type TAp73 was strongly phosphorylated in response to cisplatin treatment in MCF-10A cells, while Y99F TAp73 underwent little detectable phosphorylation (FIG. 12A). Furthermore, while IP for both wild-type and Y99F TAp73 was discovered to coimmunoprecipitate abundant ΔNp63α at baseline, cisplatin induces substantial dissociation of ΔNp63α only from wild-type, but not Y99F, TAp73 (FIG. 12A). The same result was evident following IP for p63 under these same conditions: dissociation of wildtype TAp73 from ΔNp63α was observed with 6 hours of cisplatin treatment, while essentially no dissociation of Y99F TAp73 was detected (FIG. 12A). Finally, although wild-type TAp73 expression significantly increased sensitivity of MCF-10A cells to cisplatin treatment (P<0.01), Y99F TAp73 had little, if any, effect on cisplatin sensitivity (FIG. 12B). Of note, these findings are in contrast to another point mutant the inventors investigated, Y121F TAp73, which still underwent substantial phosphorylation and dissociation from ΔNp63α and still induced cellular sensitivity following cisplatin treatment (data not shown). Thus, the inventors have demonstrated that phosphorylation of TAp73 at Y99 is essential for dissociation of the ΔNp63α/TAp73 complex and for chemosensitivity following cisplatin treatment.

### Example 9

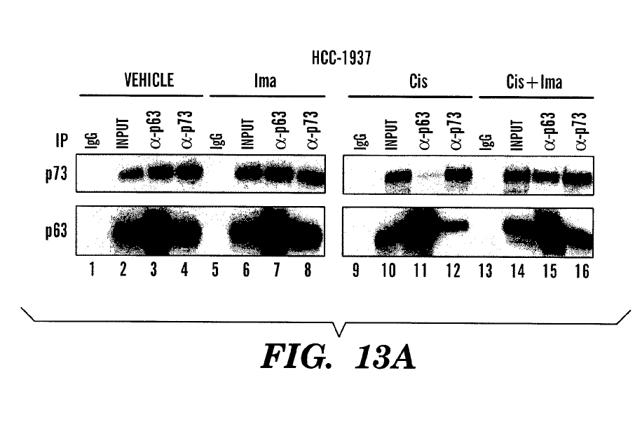

TAp73 dissociation, proapoptotic transcription, and cell death induced by cisplatin are c-Abl dependent. Given that Y99 is the major site for c-Abl phosphorylation of TAp73, the inventors have discovered c-Abl is a critical kinase required for escape of TAp73 from ΔNp63α-mediated repression. To further substantiate this discovery, the inventors assessed whether imatinib (also known as STI-571) could inhibit TAp73 activation and subsequent pro-apoptotic death induced by cisplatin. The inventors first examined dissociation of the ΔNp63α/TAp73 complex, and discovered that pretreatment of cells with imatinib substantially blocked dissociation of the endogenous ΔNp63α/TAp73 complex induced by cisplatin in breast cancer cells (FIG. 13A). Secondly, the inventors determined whether imatinib could inhibit proapoptotic transcription induced by cisplatin, by assaying for cisplatin-induced transcription of NOXA, which the inventors had previously shown to be TAp73 dependent, with or without imatinib pretreatment. The inventors discovered that pretreatment blocked induction of NOXA at 6 hours following cisplatin treatment in both HCC-1937 and MDA-MB-468 cells (FIGS. 13, B and C). This effect correlated with the ability of imatinib to block ΔNp63α/TAp73 dissociation at this same time point (FIG. 13A). Of note, imatinib treatment had no effect on baseline NOXA expression in either cell line (data not shown). Finally, imatinib treatment was discovered to result in a substantial rescue from cell death induced by cisplatin in both HCC-1937 and MDA-MB-468 cells (FIG. 13D). Imatinib treatment did not affect cell proliferation in either cell line at the dose used in these assays (data not shown). Thus, the inventors have demonstrated that cisplatin induced death in breast cancer cells expressing ΔNp63α and TAp73 occurs through a TAp73-mediated pathway involving c-Abl phosphorylation of TAp73. The inventors have discovered that this phosphorylation is required for dissociation of the ΔNp63α/TAp73 complex, for TAp73-dependent transcription, and for apoptosis following cisplatin treatment.

### Example 10

Validation of marker analysis in primary human breast cancer specimens. The inventors further validated the expression of DNp63 and TAp73 isoforms quantitatively in primary human tumor specimens. In order to determine the optimal method to measure expression of these isoforms, the inventors assessed expression of these isoforms in microdissected primary tumor specimens derived from the clinical trial described herein in the Examples. The inventors assessed different methods to quantitatively measure the transcription of DNp63 and TAp73 isoforms, and as demonstrated herein, compared two methods for real-time RT-PCR quantitation; “Taqman” (3 primer) analysis, and SYBR green-based analysis. Based on cDNA standards, the absolute value for DNp63 by Taqman versus Sybr gave an R value correlation coefficient of 0.93; whereas for TAp73 the Taqman versus Sybr correlation coefficient value was 0.94. The inventors previously demonstrated in Example 1, the RNA expression of these isoforms correlates with protein expression in tumor cells. This data together with the data of analysis of DNp63 and TAp73 isoforms in tumor-derived cell lines, demonstrates the inventors have developed robust assays for quantitation of the relevant p63 and p73 isoforms in both cultured cells and primary human tumors.

### Example 11

Correlation of DNp63/TAp73 biomarker expression and cisplatin sensitivity in vitro. The inventors next established the threshold for DNp63/TAp73 biomarker expression that reflected the biochemcially-defined cisplatin sensitivity pathway. The inventors defined biomarker positivity as a DNp63/TAp73 ratio >2, a value that corresponds to the molar ratio of DNp63 required to inactivate essentially all (>99%) pro-apoptotic TAp73 through DNp63/TAp73 hetero-tetramer formation. The inventors assessed the ability of this defined biomarker threshold to predict cisplatin sensitivity in a limited number of human breast cancer-derived cell lines. As shown below in Table 2, the positively for the biomarker correlated with cisplatin sensitivity in these cells. In particular, biomarker negativity was associated with a very low (20%) probability of sensitivity.

TABLE 2
p73/p63 Biomarker and in vitro cisplatin sensitivity (n = 12).

| IC50 for Cisplatin | Positive Biomarker |
|---|---|
| <6 uM (Sensitive) | 80% (⅘) |
| >6 uM (Insensitive) | 43% ( 3/7) |
| Biomarker | Percent Sensitive |
| Negative | 20% (⅕) |
| Positive | 57% ( 4/7) |

### Example 12

Correlation of DNp63/TAp73 biomarker expression and clinical cisplatin response. In order to test the hypothesis that DNp63/TAp73 expression correlates with the clinical response to cisplatin chemotherapy, the inventors analyzed samples from a completed clinical trial of cisplatin treatment for subjects with primary triple-negative breast carcinoma. Subjects in this trial received single-agent cisplatin every three weeks for four doses, followed by surgery. The response to cisplatin was determined by the amount of residual viable tumor detected on pathological examination of the resected tumor specimen. Using specimens from this trial, the inventors demonstrate and validate the DNp63/TAp73 biomarker as a prognisis to responsiveness to treatment in subjects who have received cisplatin treatment. The inventors determined that 75% of tumors were biomarker positive in subjects experiencing a pathologic complete response (pCR, meaning no residual viable tumor), as compared with only 33% of tumors in subjects not obtaining a pCR (Table 2). As was observed in cultured cell lines, biomarker negativity was associated with a particularly low chance of obtaining a pCR (7%) as compared with subjects whose tumors were biomarker positive (33%) (as shown in Table 3). Thus, the inventors have demonstrated that tumor-specific DNp63/TAp73 biomarker expression is a predictive marker of cisplatin sensitivity in tumor samples from human subjects in vivo. The inventors have also demonstrated that a negative p73/p63 biomarker test result is particularly useful for identifying subjects who are unlikely to benefit from cisplatin therapy. The p73/p63 biomarker can be useful as a predictor of clinical response to cisplatin in subjects with different types of tumors, for example triple-negative breast carcinoma.

TABLE 3
p73/p63 Biomarker and pathologic complete response (n = 22).

| Response | Positive Biomarker |
|---|---|
| Path. CR | 75% (¾) |
| No Path CR | 33% ( 6/18) |
| Biomarker | Percent Path CR |
| Negative | 7% ( 1/13) |
| Positive | 33% ( 3/9) |

### REFERENCES

The references cited herein and throughout the application are incorporated herein by reference.
1. Sorlie, T., C. M. Perou, R. Tibshirani, T. Aas, S. Geisler, H. Johnsen, T. Hastie, M. B. Eisen, M. van de Rijn, S. S. Jeffrey, T. Thorsen, H. Quist, J. C. Matese, P. O. Brown, D. Botstein, P. Eystein Lonning, and A. L. Borresen-Dale. 2001. Gene expression patterns of breast carcinomas distinguish tumor subclasses with clinical implications. Proc Natl Acad Sci USA 98:10869.
2. Wang, Z. C., M. Lin, L. J. Wei, C. Li, A. Miron, G. Lodeiro, L. Harris, S. Ramaswamy, D. M. Tanenbaum, M. Meyerson, J. D. Iglehart, and A. Richardson. 2004. Loss of heterozygosity and its correlation with expression profiles in subclasses of invasive breast cancers. Cancer Res 64:64.
3. Yang, A., R. Schweitzer, D. Sun, M. Kaghad, N. Walker, R. T. Bronson, C. Tabin, A. Sharpe, D. Caput, C. Crum, and F. McKeon. 1999. p63 is essential for regenerative proliferation in limb, craniofacial and epithelial development. Nature 398:714.
4. Mills, A. A., B. Zheng, X. J. Wang, H. Vogel, D. R. Roop, and A. Bradley. 1999. p63 is a p53 homologue required for limb and epidermal morphogenesis. Nature 398:708.
5. van Bokhoven, H., and H. G. Brunner. 2002. Splitting p63. Am J Hum Genet 71:1.
6. Celli, J., P. Duijf, B. C. Hamel, M. Bamshad, B. Kramer, A. P. Smits, R. Newbury-Ecob, R. C. Hennekam, G. Van Buggenhout, A. van Haeringen, C. G. Woods, A. J. van Essen, R. de Waal, G. Vriend, D. A. Haber, A. Yang, F. McKeon, H. G. Brunner, and H. van Bokhoven. 1999. Heterozygous germline mutations in the p53 homolog p63 are the cause of EEC syndrome. Cell 99:143.
7. Adriance, M. C., J. L. Inman, O. W. Petersen, and M. J. Bissell. 2005. Myoepithelial cells: good fences make good neighbors. Breast Cancer Res 7:190.
8. Carroll, D. K., J. S. Carroll, C. O. Leong, F. Cheng, M. Brown, A. A. Mills, J. S. Brugge, and L. W. Ellisen. 2006. p63 regulates an adhesion programme and cell survival in epithelial cells. Nat Cell Biol 8:551.
9. Thurfjell, N., P. J. Coates, L. Boldrup, B. Lindgren, B. Backlund, T. Uusitalo, D. Mahani, E. Dabelsteen, A. Dahlqvist, B. Sjostrom, G. Roos, B. Vojtesek, R. Nenutil, and K. Nylander. 2005. Function and importance of p63 in normal oral mucosa and squamous cell carcinoma of the head and neck. Adv Otorhinolaryngol 62:49.
10. Weber, A., U. Bellmann, F. Bootz, C. Wittekind, and A. Tannapfel. 2002. Expression of p53 and its homologues in primary and recurrent squamous cell carcinomas of the head and neck. Int J Cancer 99:22.
11. Sniezek, J. C., K. E. Matheny, M. D. Westfall, and J. A. Pietenpol. 2004. Dominant negative p63 isoform expression in head and neck squamous cell carcinoma. Laryngoscope 114:2063.
12. Massion, P. P., P. M. Taflan, S. M. Jamshedur Rahman, P. Yildiz, Y. Shyr, M. E. Edgerton, M. D. Westfall, J. R. Roberts, J. A. Pietenpol, D. P. Carbone, and A. L. Gonzalez. 2003. Significance of p63 amplification and overexpression in lung cancer development and prognosis. Cancer Res 63:7113.
13. Hu, H., S. H. Xia, A. D. Li, X. Xu, Y. Cai, Y. L. Han, F. Wei, B. S. Chen, X. P. Huang, Y. S. Han, J. W. Zhang, X. Zhang, M. Wu, and M. R. Wang. 2002. Elevated expression of p63 protein in human esophageal squamous cell carcinomas. Int J Cancer 102:580.
14. Rocco, J. W., C. O. Leong, N. Kuperwasser, M. P. DeYoung, and L. W. Ellisen. 2006. p63 mediates survival in squamous cell carcinoma by suppression of p73-dependent apoptosis. Cancer Cell 9:45.
15. DeYoung, M. P., C. J. Johannessen, C. O. Leong, W. Faquin, J. W. Rocco, and L. W. Ellisen. 2006. Tumor-specific p73 upregulation mediates p63 dependence in squamous cell carcinoma. Cancer Res (In Press).
16. Perou, C. M., T. Sorlie, M. B. Eisen, M. van de Rijn, S. S. Jeffrey, C. A. Rees, J. R. Pollack, D. T. Ross, H. Johnsen, L. A. Akslen, O. Fluge, A. Pergamenschikov, C. Williams, S. X. Zhu, P. E. Lonning, A. L. Borresen-Dale, P. O. Brown, and D. Botstein. 2000. Molecular portraits of human breast tumours. Nature 406:747.
17. Wang, X., I. Mori, W. Tang, M. Nakamura, Y. Nakamura, M. Sato, T. Sakurai, and K. Kakudo. 2002. p63 expression in normal, hyperplastic and malignant breast tissues. Breast Cancer 9:216.
18. Reis-Filho, J. S., F. Milanezi, I. Amendoeira, A. Albergaria, and F. C. Schmitt. 2003. Distribution of p63, a novel myoepithelial marker, in fine-needle aspiration biopsies of the breast: an analysis of 82 samples. Cancer 99:172.
19. Ribeiro-Silva, A., L. N. Ramalho, S. B. Garcia, D. F. Brandao, F. Chahud, and S. Zucoloto. 2005. p63 correlates with both BRCA1 and cytokeratin 5 in invasive breast carcinomas: further evidence for the pathogenesis of the basal phenotype of breast cancer. Histopathology 47:458.
20. Koker, M. M., and C. G. Kleer. 2004. p63 expression in breast cancer: a highly sensitive and specific marker of metaplastic carcinoma. Am J Surg Pathol 28:1506.
21. Harms, K., S. Nozell, and X. Chen. 2004. The common and distinct target genes of the p53 family transcription factors. Cell Mol Life Sci 61:822.
22. Yang, A., M. Kaghad, Y. Wang, E. Gillett, M. D. Fleming, V. Dotsch, N. C. Andrews, D. Caput, and F. McKeon. 1998. p63, a p53 homolog at 3q27-29, encodes multiple products with transactivating, death inducing, and dominant negative activities. Mol. Cell 2:305.
23. King, K. E., R. M. Ponnamperuma, T. Yamashita, T. Tokino, L. A. Lee, M. F. Young, and W. C. Weinberg. 2003. deltaNp63alpha functions as both a positive and a negative transcriptional regulator and blocks in vitro differentiation of murine keratinocytes. Oncogene 22:3635.
24. Barbareschi, M., L. Pecciarini, M. G. Cangi, E. Macri, A. Rizzo, G. Viale, and C. Doglioni. 2001. p63, a p53 homologue, is a selective nuclear marker of myoepithelial cells of the human breast. Am J Surg Pathol 25:1054.
25. Bergamaschi, D., M. Gasco, L. Hiller, A. Sullivan, N. Syed, G. Trigiante, I. Yulug, M. Merlano, G. Numico, A. Comino, M. Attard, O. Reelfs, B. Gusterson, A. K. Bell, V. Heath, M. Tavassoli, P. J. Farrell, P. Smith, X. Lu, and T. Crook. 2003. p53 polymorphism influences response in cancer chemotherapy via modulation of p73-dependent apoptosis. Cancer Cell 3:387.
26. Irwin, M. S., K. Kondo, M. C. Marin, L. S. Cheng, W. C. Hahn, and W. G. Kaelin, Jr. 2003. Chemosensitivity linked to p73 function. Cancer Cell 3:403.
27. Gong, J. G., A. Costanzo, H. Q. Yang, G. Melino, W. G. Kaelin, Jr., M. Levrero, and J. Y. Wang. 1999. The tyrosine kinase c-Abl regulates p73 in apoptotic response to cisplatin-induced DNA damage. Nature 399:806.
28. Agami, R., G. Blandino, M. Oren, and Y. Shaul. 1999. Interaction of c-Abl and p73alpha and their collaboration to induce apoptosis. Nature 399:809.
29. Yuan, Z. M., H. Shioya, T. Ishiko, X. Sun, J. Gu, Y. Y. Huang, H. Lu, S. Kharbanda, R. Weichselbaum, and D. Kufe. 1999. p73 is regulated by tyrosine kinase c-Abl in the apoptotic response to DNA damage. Nature 399:814.
30. Strano, S., O. Monti, N. Pediconi, A. Baccarini, G. Fontemaggi, E. Lapi, F. Mantovani, A. Damalas, G. Citro, A. Sacchi, G. Del Sal, M. Levrero, and G. Blandino. 2005. The transcriptional coactivator Yes-associated protein drives p73 gene-target specificity in response to DNA Damage. Mol Cell 18:447.
31. Mantovani, F., S. Piazza, M. Gostissa, S. Strano, P. Zacchi, R. Mantovani, G. Blandino, and G. Del Sal. 2004. Pin1 links the activities of c-Abl and p300 in regulating p73 function. Mol Cell 14:625.
32. Basu, S., N. F. Totty, M. S. Irwin, M. Sudol, and J. Downward. 2003. Akt phosphorylates the Yes-associated protein, YAP, to induce interaction with 14-3-3 and attenuation of p73-mediated apoptosis. Mol Cell 11:11.
33. Dominguez, G., J. M. Garcia, C. Pena, J. Silva, V. Garcia, L. Martinez, C. Maximiano, M. E. Gomez, J. A. Rivera, C. Garcia-Andrade, and F. Bonilla. 2006. DeltaTAp73 upregulation correlates with poor prognosis in human tumors: putative in vivo network involving p73 isoforms, p53, and E2F-1. J Clin Oncol 24:805.
34. Zaika, A. I., N. Slade, S. H. Erster, C. Sansome, T. W. Joseph, M. Pearl, E. Chalas, and U. M. Moll. 2002. DeltaNp73, a dominant-negative inhibitor of wild-type p53 and TAp73, is up-regulated in human tumors. J Exp Med 196:765.
35. Ma, X. J., R. Salunga, J. T. Tuggle, J. Gaudet, E. Enright, P. McQuary, T. Payette, M. Pistone, K. Stecker, B. M. Zhang, Y. X. Zhou, H. Varnholt, B. Smith, M. Gadd, E. Chatfield, J. Kessler, T. M. Baer, M. G. Erlander, and D. C. Sgroi. 2003. Gene expression profiles of human breast cancer progression. Proc Natl Acad Sci USA 100:5974.
36. Martin, S. J., C. P. Reutelingsperger, A. J. McGahon, J. A. Rader, R. C. van Schie, D. M. LaFace, and D. R. Green. 1995. Early redistribution of plasma membrane phosphatidylserine is a general feature of apoptosis regardless of the initiating stimulus: inhibition by overexpression of Bcl-2 and Abl. J Exp Med 182:1545.
37. Chan, W. M., W. Y. Siu, A. Lau, and R. Y. Poon. 2004. How many mutant p53 molecules are needed to inactivate a tetramer? Mol Cell Biol 24:3536.
38. Wistuba, I I, C. Behrens, S. Milchgrub, S. Syed, M. Ahmadian, A. K. Virmani, V. Kurvari, T. H. Cunningham, R. Ashfaq, J. D. Minna, and A. F. Gazdar. 1998. Comparison of features of human breast cancer cell lines and their corresponding tumors. Clin Cancer Res 4:2931.
39. Melino, G., F. Bernassola, M. Ranalli, K. Yee, W. X. Zong, M. Corazzari, R. A. Knight, D. R. Green, C. Thompson, and K. H. Vousden. 2004. p73 Induces apoptosis via PUMA transactivation and Bax mitochondrial translocation. J Biol Chem 279:8076.
40. Concin, N., K. Becker, N. Slade, S. Erster, E. Mueller-Holzner, H. Ulmer, G. Daxenbichler, A. Zeimet, R. Zeillinger, C. Marth, and U. M. Moll. 2004. Transdominant DeltaTAp73 isoforms are frequently up-regulated in ovarian cancer. Evidence for their role as epigenetic p53 inhibitors in vivo. Cancer Res 64:2449.
41. Dominguez, G., J. M. Silva, J. Silva, J. M. Garcia, A. Sanchez, A. Navarro, I. Gallego, M. Provencio, P. Espana, and F. Bonilla. 2001. Wild type p73 overexpression and high-grade malignancy in breast cancer. Breast Cancer Res Treat 66:183.
42. Saxby, A. J., A. Nielsen, C. J. Scarlett, A. Clarkson, A. Morey, A. Gill, and R. C. Smith. 2005. Assessment of HER-2 status in pancreatic adenocarcinoma: correlation of immunohistochemistry, quantitative real-time RT-PCR, and FISH with aneuploidy and survival. Am J Surg Pathol 29:1125.
43. Berry, D. A., C. Cirrincione, I. C. Henderson, M. L. Citron, D. R. Budman, L. J. Goldstein, S. Martino, E. A. Perez, H. B. Muss, L. Norton, C. Hudis, and E. P. Winer. 2006. Estrogen-receptor status and outcomes of modern chemotherapy for subjects with node-positive breast cancer. Jama 295:1658.
44. Druker, B. J. 2002. Inhibition of the Bcr-Abl tyrosine kinase as a therapeutic strategy for CML. Oncogene 21:8541.
45. Fomenkov, A., R. Zangen, Y. P. Huang, M. Osada, Z. Guo, T. Fomenkov, B. Trink, D. Sidransky, and E. A. Ratovitski. 2004. RACK1 and stratifin target DeltaNp63alpha for a proteasome degradation in head and neck squamous cell carcinoma cells upon DNA damage. Cell Cycle 3:1285.
46. Livasy, C. A., G. Karaca, R. Nanda, M. S. Tretiakova, O. I. Olopade, D. T. Moore, and C. M. Perou. 2005. Phenotypic evaluation of the basal-like subtype of invasive breast carcinoma. Mod Pathol.
47. Sorlie, T., R. Tibshirani, J. Parker, T. Hastie, J. S. Marron, A. Nobel, S. Deng, H. Johnsen, R. Pesich, S. Geisler, J. Demeter, C. M. Perou, P. E. Lonning, P. O. Brown, A. L. Borresen-Dale, and D. Botstein. 2003. Repeated observation of breast tumor subtypes in independent gene expression data sets. Proc Natl Acad Sci USA 100:8418.
48. Tassone, P., P. Tagliaferri, A. Perricelli, S. Blotta, B. Quaresima, M. L. Martelli, A. Goel, V. Barbieri, F. Costanzo, C. R. Boland, and S. Venuta. 2003. BRCA1 expression modulates chemosensitivity of BRCA1-defective HCC1937 human breast cancer cells. Br J Cancer 88:1285.
49. Turner, N., A. Tutt, and A. Ashworth. 2004. Hallmarks of ‘BRCAness’ in sporadic cancers. Nat Rev Cancer 4:814.
50. Decatris, M. P., S. Sundar, and K. J. O'Byrne. 2004. Platinum-based chemotherapy in metastatic breast cancer: current status. Cancer Treat Rev 30:53.
51. Urist, M., T. Tanaka, M. V. Poyurovsky, and C. Prives. 2004. p73 induction after DNA damage is regulated by checkpoint kinases Chk1 and Chk2. Genes Dev 18:3041.
52. Bradshaw, T. D., C. S. Matthews, J. Cookson, E. H. Chew, M. Shah, K. Bailey, A. Monks, E. Harris, A. D. Westwell, G. Wells, C. A. Laughton, and M. F. Stevens. 2005. Elucidation of thioredoxin as a molecular target for antitumor quinols. Cancer Res 65:3911.

1-62. (canceled)

63. A method of identifying the pharmacological effectiveness of a platinum-based chemotherapeutic agent for the treatment of cancer comprising subjecting a biological sample to reverse transcriptase PCR (RT-PCR) to obtain the expression level of DNp63 and TAp73, wherein a level of DNp63 at least 1.5 fold greater than the level of TAp73 indicates an increased likelihood of the pharmacological effectiveness a platinum-based chemotherapeutic agent for the treatment of cancer.

64. The method of claim 63, wherein a the level of DNp63 of at least 2.0 fold or greater than the level of TAp73 indicates an increased likelihood of the pharmacological effectiveness of a platinum-based chemotherapeutic agent for the treatment of cancer as compared to if the level of DNp63 is less than 2.0 fold of the level of at least one isoform of TAp73.

65. The method of claim 63, wherein the RT-PCR is quantitative RT-PCR.

66. The method of claim 65, wherein the quantitative RT-PCR is an automated quantitative RT-PCR to compare the expression level of DNp63 and TAp73.

67. The method of claim 63, wherein the wherein the biological sample is selected from a group consisting of; a tissue sample; a tumor sample; a tumor cell; a biopsy sample; an ex vivo cultivated sample; an ex vivo cultivated tumor sample; a surgically dissected tissue sample, a blood sample, a plasma sample, a cancer sample, a lymph fluid sample or primary ascite sample.

68. The method of claim 63, wherein the cancer is selected from a group consisting of; gastrointestinal cancer, prostate cancer, ovarian cancer, breast cancer, squamous cell carcinomas (SCC), head and neck cancer, lung cancer, non-small cell lung cancer, cancer of the nervous system, brain cancer, bone-marrow cancer, bone cancer, kidney cancer, retina cancer, skin cancer, bladder cancer, colon cancer, esophageal cancer, testicular cancer, cervical cancer, liver cancer, renal cancer, pancreatic cancer, genital-urinary cancer, gastrointestinal, gum cancer, tongue cancer, kidney cancer, nasopharynx cancer, stomach cancer, endometrial cancer and bowel tumor cell cancer.

69. The method of claim 63, wherein the cancer is selected from the group consisting of: breast cancer of the a triple-negative subtype breast cancer; a cancer which lacks the expression of estrogen receptor (ER), the progesterone receptor (PR) and lacks Her-2 expression; squamous cell carcinomas (SCC), or squamous cell carcinomas (SCC) of the head, neck lung and esophagus; prostate cancer.

70. The method of claim 63, wherein a platinum-based chemotherapeutic agent selected from the group consisting of: cisplatin, cisplatin compound, or a metabolite or derivative or analogue thereof.

71. The method of claim 70, wherein the cisplatin derivative is selected from a group comprising carboplatin and oxaliplatin or derivatives thereof.


SEQUENCE LISTING (29 sequences)

SEQ ID NO: 1 - DNA, 2234 bp, Homo sapiens
aggggacgcagcgaaaccggggcccgcgccaggccagccgggacggacgccgatgcccggggctgcgacggctgcagagcgagctgccctcggaggccggcgtggggaagatggcccagtccaccgccacctcccctgatgggggcaccacgtttgagcacctctggagctctctggaaccagacagcacctacttcgaccttccccagtcaagccgggggaataatgaggtggtgggcggaacggattccagcatggacgtcttccacctggagggcatgactacatctgtcatggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcactgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgagtcacctacagcccccgtcctacgggccggtcctctcgcccatgaacaaggtgcacgggggcatgaacaagctgccctccgtcaaccagctggtgggccagcctcccccgcacagttcggcagctacacccaacctggggcccgtgggccccgggatgctcaacaaccatggccacgcagtgccagccaacggcgagatgagcagcagccacagcgcccagtccatggtctcggggtcccactgcactccgccacccccctaccacgccgaccccagcctcgtcagttttttaacaggattggggtgtccaaactgcatcgagtatttcacctcccaagggttacagagcatttaccacctgcagaacctgaccattgaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactgagggcctcgcctggctgcagcctgcgccaccgcccagagacccaagctgcctcccctctccttcctgtgtgtccaaaactgcctcaggaggcaggaccttcgggctgtgcccggggaaaggcaaggtccggcccatccccaggcacctcacaggccccaggaaaggcccagccaccgaagccgcctgtggacagcctgagtcacctgcagaacc

SEQ ID NO: 2 - protein, 636 aa, Homo sapiens
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVSFLTGLGCPNCIEYFTSQGLQSIYHLQNLTIEDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 3 - DNA, 2211 bp, Homo sapiens
ggattcagccagttgacagaactaagggagatgggaaaagcgaaaatgccaacaaacggcccgcatgttccccagcatcctcggctcctgcctcactagctgcggagcctctcccgctcggtccacgctgccgggcggccacgaccgtgacccttcccctcgggccgcccagatccatgcctcgtcccacgggacaccagttccctggcgtgtgcagaccccccggcgcctaccatgctgtacgtcggtgaccccgcacggcacctcgccacggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcactgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgagtcacctacagcccccgtcctacgggccggtcctctcgcccatgaacaaggtgcacgggggcatgaacaagctgccctccgtcaaccagctggtgggccagcctcccccgcacagttcggcagctacacccaacctggggcccgtgggccccgggatgctcaacaaccatggccacgcagtgccagccaacggcgagatgagcagcagccacagcgcccagtccatggtctcggggtcccactgcactccgccacccccctaccacgccgaccccagcctcgtcagttttttaacaggattggggtgtccaaactgcatcgagtatttcacctcccaagggttacagagcatttaccacctgcagaacctgaccattgaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactgagggcctcgcctggctgcagcctgcgccaccgcccagagacccaagctgcctcccctctccttcctgtgtgtccaaaactgcctcaggaggcaggaccttcgggctgtgcccggggaaaggcaaggtccggcccatccccaggcacctcacaggccccaggaaaggcccagccaccgaagccgcctgtggacagcctgagtcacctgcagaacc

SEQ ID NO: 4 - protein, 587 aa, Homo sapiens
MLYVGDPARHLATAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVSFLTGLGCPNCIEYFTSQGLQSIYHLQNLTIEDLGALKIPEQYRMTIWRGLQDLKQGHDYSTAQQLLRSSNAATISIGGSGELQRQRVMEAVHFRVRHTITIPNRGGPGGGPDEWADFGFDLPDCKARKQPIKEEFTEAEIH

SEQ ID NO: 5 - DNA, 1926 bp, Homo sapiens
atgtcccagagcacacagacaaatgaattcctcagtccagaggttttccagcatatctgggattttctggaacagcctatatgttcagttcagcccattgacttgaactttgtggatgaaccatcagaagatggtgcgacaaacaagattgagattagcatggactgtatccgcatgcaggactcggacctgagtgaccccatgtggccacagtacacgaacctggggctcctgaacagcatggaccagcagattcagaacggctcctcgtccaccagtccctataacacagaccacgcgcagaacagcgtcacggcgccctcgccctacgcacagcccagctccaccttcgatgctctctctccatcacccgccatcccctccaacaccgactacccaggcccgcacagtttcgacgtgtccttccagcagtcgagcaccgccaagtcggccacctggacgtattccactgaactgaagaaactctactgccaaattgcaaagacatgccccatccagatcaaggtgatgaccccacctcctcagggagctgttatccgcgccatgcctgtctacaaaaaagctgagcacgtcacggaggtggtgaagcggtgccccaaccatgagctgagccgtgaattcaacgagggacagattgcccctcctagtcatttgattcgagtagaggggaacagccatgcccagtatgtagaagatcccatcacaggaagacagagtgtgctggtaccttatgagccaccccaggttggcactgaattcacgacagtcttgtacaatttcatgtgtaacagcagttgtgttggagggatgaaccgccgtccaattttaatcattgttactctggaaaccagagatgggcaagtcctgggccgacgctgctttgaggcccggatctgtgcttgcccaggaagagacaggaaggcggatgaagatagcatcagaaagcagcaagtttcggacagtacaaagaacggtgatggtacgaagcgcccgtttcgtcagaacacacatggtatccagatgacatccatcaagaaacgaagatccccagatgatgaactgttatacttaccagtgaggggccgtgagacttatgaaatgctgttgaagatcaaagagtccctggaactcatgcagtaccttcctcagcacacaattgaaacgtacaggcaacagcaacagcagcagcaccagcacttacttcagaaacagacctcaatacagtctccatcttcatatggtaacagctccccacctctgaacaaaatgaacagcatgaacaagctgccttctgtgagccagcttatcaaccctcagcagcgcaacgccctcactcctacaaccattcctgatggcatgggagccaacattcccatgatgggcacccacatgccaatggctggagacatgaatggactcagccccacccaggcactccctcccccactctccatgccatccacctcccactgcacacccccacctccgtatcccacagattgcagcattgtcagtttcttagcgaggttgggctgttcatcatgtctggactatttcacgacccaggggctgaccaccatctatcagattgagcattactccatggatgatctggcaagtctgaaaatccctgagcaatttcgacatgcgatctggaagggcatcctggaccaccggcagctccacgaattctcctccccttctcatctcctgcggaccccaagcagtgcctctacagtcagtgtgggctccagtgagacccggggtgagcgtgttattgatgctgtgcgattcaccctccgccagaccatctctttcccaccccgagatgagtggaatgacttcaactttgacatggatgctcgccgcaataagcaacagcgcatcaaagaggagggggagtga

SEQ ID NO: 6 - protein, 641 aa, Homo sapiens
MSQSTQTNEFLSPEVFQHIWDFLEQPICSVQPIDLNFVDEPSEDGATNKIEISMDCIRMQDSDLSDPMWPQYTNLGLLNSMDQQIQNGSSSTSPYNTDHAQNSVTAPSPYAQPSSTFDALSPSPAIPSNTDYPGPHSFDVSFQQSSTAKSATWTYSTELKKLYCQIAKTCPIQIKVMTPPPQGAVIRAMPVYKKAEHVTEVVKRCPNHELSREFNEGQIAPPSHLIRVEGNSHAQYVEDPITGRQSVLVPYEPPQVGTEFTTVLYNFMCNSSCVGGMNRRPILIIVTLETRDGQVLGRRCFEARICACPGRDRKADEDSIRKQQVSDSTKNGDGTKRPFRQNTHGIQMTSIKKRRSPDDELLYLPVRGRETYEMLLKIKESLELMQYLPQHTIETYRQQQQQQHQHLLQKQTSIQSPSSYGNSSPPLNKMNSMNKLPSVSQLINPQQRNALTPTTIPDGMGANIPMMGTHMPMAGDMNGLSPTQALPPPLSMPSTSHCTPPPPYPTDCSIVSFLARLGCSSCLDYFTTQGLTTIYQIEHYSMDDLASLKIPEQFRHAIWKGILDHRQLHEFSSPSHLLRTPSSASTVSVGSSETRGERVIDAVRFTLRQTISFPPRDEWNDFNFDMDARRNKQQRIKEEGE

SEQ ID NO: 7 - DNA, 1761 bp, Homo sapiens
atgttgtacctggaaaacaatgcccagactcaatttagtgagccacagtacacgaacctggggctcctgaacagcatggaccagcagattcagaacggctcctcgtccaccagtccctataacacagaccacgcgcagaacagcgtcacggcgccctcgccctacgcacagcccagctccaccttcgatgctctctctccatcacccgccatcccctccaacaccgactacccaggcccgcacagtttcgacgtgtccttccagcagtcgagcaccgccaagtcggccacctggacgtattccactgaactgaagaaactctactgccaaattgcaaagacatgccccatccagatcaaggtgatgaccccacctcctcagggagctgttatccgcgccatgcctgtctacaaaaaagctgagcacgtcacggaggtggtgaagcggtgccccaaccatgagctgagccgtgaattcaacgagggacagattgcccctcctagtcatttgattcgagtagaggggaacagccatgcccagtatgtagaagatcccatcacaggaagacagagtgtgctggtaccttatgagccaccccaggttggcactgaattcacgacagtcttgtacaatttcatgtgtaacagcagttgtgttggagggatgaaccgccgtccaattttaatcattgttactctggaaaccagagatgggcaagtcctgggccgacgctgctttgaggcccggatctgtgcttgcccaggaagagacaggaaggcggatgaagatagcatcagaaagcagcaagtttcggacagtacaaagaacggtgatggtacgaagcgcccgtttcgtcagaacacacatggtatccagatgacatccatcaagaaacgaagatccccagatgatgaactgttatacttaccagtgaggggccgtgagacttatgaaatgctgttgaagatcaaagagtccctggaactcatgcagtaccttcctcagcacacaattgaaacgtacaggcaacagcaacagcagcagcaccagcacttacttcagaaacagacctcaatacagtctccatcttcatatggtaacagctccccacctctgaacaaaatgaacagcatgaacaagctgccttctgtgagccagcttatcaaccctcagcagcgcaacgccctcactcctacaaccattcctgatggcatgggagccaacattcccatgatgggcacccacatgccaatggctggagacatgaatggactcagccccacccaggcactccctcccccactctccatgccatccacctcccactgcacacccccacctccgtatcccacagattgcagcattgtcagtttcttagcgaggttgggctgttcatcatgtctggactatttcacgacccaggggctgaccaccatctatcagattgagcattactccatggatgatctggcaagtctgaaaatccctgagcaatttcgacatgcgatctggaagggcatcctggaccaccggcagctccacgaattctcctccccttctcatctcctgcggaccccaagcagtgcctctacagtcagtgtgggctccagtgagacccggggtgagcgtgttattgatgctgtgcgattcaccctccgccagaccatctctttcccaccccgagatgagtggaatgacttcaactttgacatggatgctcgccgcaataagcaacagcgcatcaaagaggagggggagtga

SEQ ID NO: 8 - protein, 586 aa, Homo sapiens
MLYLENNAQTQFSEPQYTNLGLLNSMDQQIQNGSSSTSPYNTDHAQNSVTAPSPYAQPSSTFDALSPSPAIPSNTDYPGPHSFDVSFQQSSTAKSATWTYSTELKKLYCQIAKTCPIQIKVMTPPPQGAVIRAMPVYKKAEHVTEVVKRCPNHELSREFNEGQIAPPSHLIRVEGNSHAQYVEDPITGRQSVLVPYEPPQVGTEFTTVLYNFMCNSSCVGGMNRRPILIIVTLETRDGQVLGRRCFEARICACPGRDRKADEDSIRKQQVSDSTKNGDGTKRPFRQNTHGIQMTSIKKRRSPDDELLYLPVRGRETYEMLLKIKESLELMQYLPQHTIETYRQQQQQQHQHLLQKQTSIQSPSSYGNSSPPLNKMNSMNKLPSVSQLINPQQRNALTPTTIPDGMGANIPMMGTHMPMAGDMNGLSPTQALPPPLSMPSTSHCTPPPPYPTDCSIVSFLARLGCSSCLDYFTTQGLTTIYQIEHYSMDDLASLKIPEQFRHAIWKGILDHRQLHEFSSPSHLLRTPSSASTVSVGSSETRGERVIDAVRFTLRQTISFPPRDEWNDFNFDMDARRNKQQRIKEEGE

SEQ ID NO: 9 - DNA, 21 bp, Artificial Sequence
  Description of Artificial Sequence Synthetic primer
gcaccacgtttgagcacctct

SEQ ID NO: 10 - DNA, 21 bp, Artificial Sequence
  Description of Artificial Sequence Synthetic primer
gcagattgaactgggccatga

SEQ ID NO: 11 - DNA, 20 bp, Artificial Sequence
  Description of Artificial Sequence Synthetic primer
ggaaaacaatgcccagactc

SEQ ID NO: 12 - DNA, 20 bp, Artificial Sequence
  Description of Artificial Sequence Synthetic primer
gtggaatacgtccaggtggc

SEQ ID NO: 13 - DNA, 20 bp, Artificial Sequence
  Description of Artificial Sequence Synthetic primer
tgctgatggactgccaaaaa

SEQ ID NO: 14 - DNA, 24 bp, Artificial Sequence
  Description of Artificial Sequence Synthetic primer
tggctgctcactactatccagaac

SEQ ID NO: 15 - DNA, 23 bp, Artificial Sequence
  Description of Artificial Sequence Synthetic primer
cccttactggcttacctcctcat

SEQ ID NO: 16 - DNA, 23 bp, Artificial Sequence
  Description of Artificial Sequence Synthetic primer
cccttaccctggctactcataca

SEQ ID NO: 17 - DNA, 16 bp, Artificial Sequence
  Description of Artificial Sequence Synthetic probe
attttgcgacatcttt

SEQ ID NO: 18 - DNA, 16 bp, Artificial Sequence
  Description of Artificial Sequence Synthetic probe
cagcctactctccttg

SEQ ID NO: 19 - DNA, 21 bp, Artificial Sequence
  Description of Artificial Sequence Synthetic oligonucleotide
ggattccagcatggacgtctt

SEQ ID NO: 20 - DNA, 21 bp, Artificial Sequence
  Description of Artificial Sequence Synthetic oligonucleotide
gagtggaatgacttcaacttt

SEQ ID NO: 21 - DNA, 22 bp, Artificial Sequence
  Description of Artificial Sequence Synthetic oligonucleotide
gggtgagcgtgttattgatgct

SEQ ID NO: 22 - DNA, 20 bp, Artificial Sequence
  Description of Artificial Sequence Synthetic primer
aagatggtgcgacaaacaag

SEQ ID NO: 23 - DNA, 20 bp, Artificial Sequence
  Description of Artificial Sequence Synthetic primer
agagagcatcgaaggtggag

SEQ ID NO: 24 - DNA, 20 bp, Artificial Sequence
  Description of Artificial Sequence Synthetic primer
caaacggcccgcatgttccc

SEQ ID NO: 25 - DNA, 20 bp, Artificial Sequence
  Description of Artificial Sequence Synthetic primer
ttgaactgggccgtggcgag

SEQ ID NO: 26 - DNA, 21 bp, Artificial Sequence
  Description of Artificial Sequence Synthetic primer
cacccagaagactgtggatgg

SEQ ID NO: 27 - DNA, 21 bp, Artificial Sequence
  Description of Artificial Sequence Synthetic primer
gtctacatggcaactgtgagg

SEQ ID NO: 28 - DNA, 21 bp, Artificial Sequence
  Description of Artificial Sequence Synthetic primer
agctgtgctcgcgctactctc

SEQ ID NO: 29 - DNA, 21 bp, Artificial Sequence
  Description of Artificial Sequence Synthetic primer
cacacggcaggcatactcatc